(19) United States
(12) Patent Application Publication
(10) Pub. No.: US 2021/0395773 A1
(43) Pub. Date: Dec. 23, 2021

(54) IN VIVO CONTROLLED COMBINATION THERAPY FOR TREATMENT OF CANCER

(71) Applicant: PRECIGEN, INC., Blacksburg, VA (US)

(72) Inventors: John Andrew BARRETT, Groton, MA (US); Francois LEBEL, Bethesda, MA (US)

(73) Assignee: PRECIGEN, INC., Blacksburg, VA (US)

(21) Appl. No.: 17/255,618

(22) PCT Filed: Jun. 27, 2019
(86) PCT No.: PCT/US2019/039568

§ 371 Date: Dec. 23, 2020

Related U.S. Application Data
(60) Provisional application No. 62/690,552, filed on Jun. 27, 2018.
(60) Provisional application No. 62/834,685, filed on Apr. 16, 2019.
(60) Provisional application No. 62/854,771, filed on May 30, 2019.

(51) Int. Cl. C12N 15/86; C07K 16/28; C07K 14/54; C07K 14/705; A61K 31/573

(52) U.S. Cl. CPC C12N 15/86; C07K 16/2818; A61P 35/00; C07K 14/70567; A61K 31/573; C07K 14/5434

(57) ABSTRACT

Disclosed herein are improved methods for treatment of brain cancer (such as glioma/glioblastoma) via ligand-inducible gene-switch controlled in vivo expression of an immunomodulator (i.e., IL-12) in combination with one or more other immunomodulators (i.e., an immune cell check point inhibitor; e.g., such as a PD-1 inhibitor or a PD-1 binder.

### FIELD OF THE INVENTION

The field of the invention is cancer immunotherapy.

### BACKGROUND

Interleukin-12 (IL-12) is a heterodimeric (IL-12-p70; IL-12-p35/p40) pro-inflammatory cytokine that induces the local and systemic production of IL-12, initiates a cytokine cascade resulting in downstream endogenous interferon-γ (IFN-γ), and via these signaling pathways activates both innate (i.e, NK cells) and adaptive (i.e, cytotoxic T lymphocytes) immunities. The adaptive immune system induces T cells to change from a naïve phenotype to an effector functional type or a memory type. The Th1/Th2 phenotype reflects the result of naïve T cell activation. IL-12 also acts to remodel the tumor microenvironment (TME) and has anti-angiogenic effects wherein it seemly inhibits pathological neovascularization. IL-12 binds to the IL-12 receptor (IL-12R), which is a heterodimeric receptor formed by IL-12R-β1 and IL-12R-β2. The receptor complex is primarily expressed by T cells, but also other lymphocyte subpopulations have been found to be responsive to IL-12.

IL-12 is a candidate for tumor immunotherapy in humans because it provides functions in bridging innate and adaptive immunity. Indeed, IL-12 has proven effective in animal models of tumor therapy. However, clinically severe side effects were frequently associated with systemic administration of IL-12 in human therapeutic studies. Despite such hurdles, however, IL-12 continues to be of significant interest for use in human (clinical) oncology, particularly because its full therapeutic potential when used by itself or in combination with other onco-therapeutic compounds and methods of treatment, or in particular via local production rather than systemic administration, has not been fully investigated, much less realized.

Observed immune cell infiltration of glioblastomas has been highly variable and is thought to be driven by the genetic composition and mutational load of a tumor (Beier et al. 2012, Doucette et al. 2013). Moreover, due to the specificity and efficiency of cytotoxic T-cells (CD8+), activation of these cells by local, controlled (i.e., regulatable) production of IL-12, is a particularly attractive therapeutic option as this may spare normal brain cells while also minimizing systemic toxicity. Furthermore, it has also been shown, in an orthotopic mouse model, that survival and reduction of tumor size (i.e., tumor cell killing) was significantly enhanced by combining an immune cell checkpoint inhibitor along with controlled administration of IL-12 (Barrett et al. 2016).

An encouraging example of a combination immunotherapy approach to treating glioblastoma is a study of the safety and activity of nivolumab (programmed cell death protein 1 [PD-1] checkpoint inhibitor) monotherapy and nivolumab in combination with ipilimumab (anti cytotoxic T lymphocyte associated antigen 4 (CTLA-4) antibody) in patients with recurrent disease (Reardon et al. 2016). CTLA-4 and PD-1 are both members of the extended CD28/CTLA-4 family of T cell regulators. PD-1 is expressed on the surface of activated T cells, B cells and macrophages. PD-1 (CD279; Uniprot Q15116) has two ligands, PD-L1 (B7-H1, CD274) and PD-L2 (B7-DC, CD273), which are members of the B7 family.

Tumor immune-stimulation via IL-12 coupled with one or more immune modulators such as PD-1 binders or PD-1 inhibitors should result in enhanced efficacy over monotherapy. There remains an unmet need for combination regimens of IL-12 and immune modulators such as immune checkpoint inhibitors that will provide may substantially improved clinical results in the regression of cancerous tumors, such as glioblastoma, while also substantially improving the long-term survival rates. An unmet need also exists for a solution to mitigate, avoid or limit systemic toxicities. The controlled production of IL-12 may increase patient tolerance to immune modulators such as checkpoint inhibitors, in particular, PD-1 inhibitors.

### SUMMARY OF THE INVENTION

In some embodiments the invention provides a method of treating a subject having cancer by administering to a subject a Ad-RTS-hIL-12 viral vector comprising a first polynucleotide encoding a polypeptide which is at least 85% identical to wild type human IL-12 p40, a second polynucleotide encoding a polypeptide which is at least 85% identical to wild type human IL-12 p35, a third polynucleotide encoding a VP-16 transactivation domain-retinoic acid-X-receptor fusion protein (VP-16-RXR) and a fourth polynucleotide encoding a Gal4 DNA binding domain and an ecdysone receptor (EcR) binding domain fusion protein (Gal4-EcR), wherein the VP-16-RXR fusion protein and the Gal4-EcR fusion protein form a ligand dependent transcription factor complex; and a diacylhydrazine ligand that activates the ligand-dependent transcription factor complex. In some embodiments, the subject having cancer is further administered with one or more immune modulators.

In some embodiments, the first polynucleotide and the second polynucleotide is joined by a first linker. In some embodiments, the third polynucleotide and the fourth polynucleotide is joined by a second linker. In some embodiments, the first linker and/or the second linker is an internal ribosome entry site (IRES) sequence. In some embodiments, the first linker and the second linker are different IRES sequences.

In some embodiments, the vector is a replication-deficient adenoviral vector. In some embodiments, the vector is administered locally to the site of the tumor. In some embodiments, the vector is administered intratumorally or to a lymph node associated with the tumor.

In some embodiments, the diacylhydrazine ligand is administered orally or parenterally.

In some embodiments, immune modulator is administered orally or parenterally.

In some embodiments, a first dose of the vector is administered concurrently with the one or more doses of the immune modulator. In some embodiments, a first dose of the vector is administered at a period of time after the administration of one or more doses of the immune modulator. In some embodiments, one or more doses of the immune modulator is administered to the subject at about 5 to 10 days prior to the administration of the vector. In some embodiments, one or more doses of the immune checkpoint inhibitor is administered to the subject about 7 days prior to the administration of the vector. In some embodiments, a first dose of the vector is administered at a period of time before the administration of one or more doses of the immune modulator.

In some embodiments, one or more subsequent doses of the immune modulator is administered after the administration of the vector. In some embodiments, one or more subsequent doses of the immune modulator are administered to the subject at least 7 days after administration of the vector. In some embodiments, one or more subsequent doses of the immune modulator are administered to the subject within 7 to 28 days after the administration of the vector. In some embodiments, one or more subsequent doses of the immune modulator are administered to the subject at about 15 days after the administration of the vector.

In some embodiments, subsequent doses of the immune modulator are administered once every two weeks after administration of a first subsequent dose of the immune modulator. In some embodiments, subsequent doses of the immune modulator are administered once every four weeks after the administration of the first subsequent dose of the immune modulator.

In some embodiments, the initial dose of the vector and the initial dose of the diacylhydrazine ligand is administered concurrently or sequentially. In some embodiments, the initial dose of the diacylhydrazine ligand is administered at a period of time after the initial dose of the vector. In some embodiments, the initial dose of the diacylhydrazine ligand is administered at a period of time prior to the initial dose of the vector. In some embodiments, the initial dose of the diacylhydrazine ligand is administered at about 1 to 5 hours prior to the administration of the vector.

In some embodiments, one or more subsequent doses of the diacylhydrazine ligand are administered once daily after the administration of the initial dose. In some embodiments, the subsequent daily doses of the diacylhydrazine ligand are administered for a period of time of about 3-28 days. In some embodiments, the subsequent daily doses of the diacylhydrazine ligand are administered for a period of time of about 14 days.

In some embodiments, the vector is administered at a unit dose of about 1×10¹¹, 2×10¹¹, 3×10¹¹, 4×10¹¹, 5×10¹¹, 6×10¹¹, 7×10¹¹, 8×10¹¹, 9×10¹¹, or 1×10¹², or 2×10¹² viral particles (vp). In some embodiments, the vector is administered at a dose of about 2×10¹¹ vp.

In some embodiments, the diacylhydrazine ligand is (R)—N′-(3,5-dimethylbenzoyl)-N′-(2,2-dimethylhexan-3-yl)-2-ethyl-3-methoxybenzohydrazide. In some embodiments, the diacylhydrazine ligand is administered at a unit daily dose of about 1 mg to about 120 mg.

In some embodiments, the diacylhydrazine ligand is administered at unit daily dose of about 5, 10, 15, 20, 25, 30, 35, 40, 50, 60, 70, 80, 90, 100 or 120 mg. In some embodiments, the diacylhydrazine ligand is administered at a unit daily dose of about 5 mg. In some embodiments, the diacylhydrazine ligand is administered at a unit daily dose of about 10 mg. In some embodiments, the diacylhydrazine ligand is administered at a unit daily dose of about 15 mg. In some embodiments, the diacylhydrazine ligand is administered daily at a unit daily dose of about 20 mg.

In some embodiments, the immune modulator is an immune checkpoint inhibitor, a chemotherapy, a radiation, a molecule that stimulates T cells an/or NK cells, a cytokine, an antigen-specific binder, a Tcell, a NK cell, a cell expressing an introduced chimeric antigen receptor or a cell expressing an introduced T cell receptor. In some embodiments, the immune modulator is a CD38 binder, a SLAMF-7 (CSI) binder, a CD96 binder, a DNAM-1 (CD226) binder, a NKG2A binder, a NKG2D binder, a MGN-3 binder, a Nectin-1 binder, a Nectin-2 binder, a dendritic cell vaccine, a tumor-associated peptide vaccine (TUMAP) vaccine, an oncofetal antigen vaccine, a viral vaccine, an immunostimulant adjuvant, a LRS binder, a C-type lectin binder, an IFN-gamma stimulator, a blocker and/or inhibitor of TGF-beta, an IDO inhibitor, cytokines such as IL-2, IL-7, IL-9, IL-15, IL-21, a CD25 binder, a TLR binder, a TLR2 binder, a IDO1 binder, a TDO binder, a CD39 binder, a CD73 binder, a Galectin 9 binder, a HMGB1 binder, a phosphatidyl serine binder, a CECAM-1 binder, a CD40 binder, a CD40L binder, an OX40 binder, a 4-1BB (CD137) binder, a 4-1BBL (CD137L) binder, a glucocorticoid-induced TNFR family-related protein (GITR) binder, GITR ligand (GITRL) binder, a CD27 binder or a killer inhibitory receptor (KTR) binder. In some embodiments, the immune checkpoint inhibitor is a PD-1 binder, a PD-L1 binder, a CTLA-4 binder, a V-domain immunoglobulin suppressor of T cell activation (VISTA) binder, a TIM-3 binder, a TIM-3 ligand binder, a LAG-3 binder, a T-cell immunoreceptor with Ig and ITIM domains (TIGIT) binder, a B- and T-cell attenuator (BTLA) binder, a B7-H3 binder, a TGFbeta and PD-L1 bispecific binder or a PD-L1 and B7.1 bispecific binder.

In some embodiments, the PD-1 binder is nivolumab (MDX 1106), pembrolizumab (MK-3475), pidilizumab (CT-011), MEDI-0680 (AMP-514), PDR-001, cemiplimab-rwlc (REGN2810), AMP-224, STI-A1110, AUNP-12, or BGB-A317. In some embodiments, the PD-1 binder is nivolumab (MDX 1106). In some embodiments, nivolumab (MDX 1106) is administered at one or more doses of about 0.5 mg/kg to about 7 mg/kg. In some embodiments, nivolumab (MDX 1106) is administered at a dose of about 1 mg/kg. In some embodiments, nivolumab (MDX 1106) is administered at a dose of about 3 mg/kg. In some embodiments, nivolumab (MDX 1106) is administered at one or more flat doses of about 30 mg to about 500 mg. In some embodiments, nivolumab (MDX 1106) is administered at a flat dose of about 240 mg. In some embodiments, nivolumab (MDX 1106) is administered at a flat dose of about 480 mg.

In some embodiments, the PD-1 binder is cemiplimab-rwlc (REGN-2810). In some embodiments, cemiplimab-rwlc (REGN-2810) is administered at a dose of about 0.5 mg/kg to about 6 mg/kg.

In some embodiments, the PD-1 binder is administered intravenously.

In some embodiments, the method of the invention the subject having cancer is further administrated with an effective amount of a corticosteroid. In some embodiments, the corticosteroid is dexamethasone.

In some embodiments, the subject has never previously been administered with corticosteroid prior to the administration of the diacylhydrazine ligand. In some embodiments, the subject has not previously been administered with corticosteroid within 4 weeks prior to the administration of the diacylhydrazine ligand. In some embodiments, the subject has previously been administered corticosteroid prior to the administration of the diacylhydrazine ligand. In some embodiments, the subject has previously been administered corticosteroid within 4 weeks prior to the administration of the diacylhydrazine. In some embodiments, the corticosteroid is administered during the administration of the diacylhydrazine ligand. In some embodiments, the cumulative dose of corticosteroid during the administration of diacylhydrazine ligand is less than or equal to about 20 mg. In some embodiments, the corticosteroid is administered intravenously or orally.

In some embodiments, the cancer is a primary tumor. In some embodiments, the cancer is a metastatic tumor. In some embodiments, the cancer is a recurrent cancer or a progressive cancer. In some embodiments, the cancer is a solid tumor. In some embodiments, the cancer is a tumor of the central nervous system, a glioma tumor, renal cancer tumor, an ovarian cancer tumor, a head and neck cancer tumor, a liver cancer tumor, a pancreatic cancer tumor, a gastric cancer tumor, an esophageal cancer tumor, a bladder cancer tumor, a ureter cancer tumor, a renal pelvis cancer tumor, a urothelial cell cancer tumor, a urogenital cancer tumor, a cervical cancer tumor, a endometrial cancer tumor, a penile cancer tumor, a thyroid cancer tumor, or a prostate cancer tumor, a breast cancer tumor, a melanoma tumor, a glioma tumor, a colon cancer tumor, a lung cancer tumor, a sarcoma cancer tumor, or a squamous cell tumor, or a prostate cancer tumor.

In some embodiments, the tumor of the central nervous system is a chordoma, a craniopharyngioma, a gangliocytoma, a glomus jugulare, a meningioma, a pineocytoma, a pineoblastoma, a pituitary adenoma, a glioma, a astrocytoma, a pilocytic astrocytoma, a “diffuse” astrocytoma, a anaplastic astrocytoma, a ependymoma, a anaplastic ependymoma, a glioblastoma multiforme (GBM), a medulloblatoma, a oligodendroglioma, a pure oligodendroglioma, a anaplastic oligodendroglioma, a anaplastic oliogoastrocytoma ganglioglioma, a acoustic neuroma (schwannoma), a vestibular schwannoma, a brain metastases, a choroid plexus carcinoma, a embryonal tumor, a germ cell tumor, a dysembryoplastic neuroepithelial tumor (DNETs), a choriocarcinoma, teratoma, a Yolk sac tumor (endodermal sinus tumor), a primary CNS lymphoma, a hemangioblastoma, a rhabdoid tumor, a glioma, a adenoma, a blastoma, a carcinoma, a sarcoma, a pineal tumor, a medulloblastoma, a medulloepithelioma, a atypical teratoid/rhabdoid tumor (ATRT), a pilocytic astrocytoma, a subependymal giant cell astrocytoma (SEGAs), a diffuse astrocytoma, a pleomorphic xanthoastrocytoma (PXAs), a optical glioma, a brain stem glioma, a focal brain stem glioma, diffuse midline glioma, a diffuse intrinsic pontine glioma (DIPGs), a midline tumor, a ganglioglioma, a craniopharyngioma, a pineal region tumor, a glioblastoma, a anaplastic astrocytoma, a embryonal tumor with multilayered rosettes, a primitive neuroectodermal tumor (PNETs), a pineoblastoma, a germinoma, a choroid plexus papilloma, a choroid plexus carcinoma, a acoustic neuroma, a neuroblastoma, a pituitary tumor, a high grade glioma, a medulloblastoma (MB), a neuroblastoma (NB), a Ewing sarcoma (EWS) or a osteosarcoma. In some embodiments, the tumor of the central nervous system is a glioma, glioblastoma, glioblastoma multiforme, anaplastic oliogoastrocytoma, a diffuse intrinsic pontine glioma (DIPG) or a mid-line tumor. In some embodiments, the glioblastoma is a recurrent glioblastoma.

In some embodiments, the glioblastoma is a progressive glioblastoma. In some embodiments, the glioma is a malignant glioma.

In some embodiments, the subject is a human. In some embodiments, the subject is a pediatric patient or an adult patient. In some embodiments, method of the invention produces an abscopal effect in the subject.

### DESCRIPTION OF THE DRAWINGS

The invention can be more completely understood with reference to the following drawings.

FIG. 1 depicts a plasmid map for a regulated promoter expression system for a bicistronic transcript encoding human IL-12 (hIL-12).

FIG. 2 depicts a plasmid map for a regulated promoter expression system for IL-21.

FIG. 3 depicts shows the structure of the vector Ad-RTS-hIL-12 (rAd.RheoIL12) in which part or all the E1 and E3 regions have been deleted and gene switch components (sometimes designated “RTS” for “RHEOSWITCH THERAPEUTIC SYSTEM”) components replace the E1 region. The box labeled “IL12” represents the IL-12p40 and IL-12p35 coding sequences separated by an IRES (Internal Ribosome Entry Site) polynucleotide sequence.

FIG. 4A is a schematic representation of the generation of orthotopic GL-261 glioma mice.

FIG. 4B depicts the dosing regimen of anti-PD-1-specific mAb CD279 (clone RMP1-14, BioXCell, cat #BP0146, West Lebanon, N.H.) in orthotopic GL-261 glioma mice.

FIG. 5 depicts the veledimex levels at 24 hours post-veledimex treatment in both normal (control) C57BL/6 control mice and mice bearing orthotopic GL-261 glioma.

FIG. 6 depicts overall survival in mice that received Ad-RTS-mIL-12+veledimex+anti-PD-1, Ad-RTS-mIL-12+veledimex monotherapy, or anti-PD-1 monotherapy.

FIG. 7 depicts the change in reduction in body weight of mice that received Ad-RTS-mIL-12+veledimex+anti-PD-1, Ad-RTS-mIL-12+veledimex monotherapy, or anti-PD-1 monotherapy.

FIG. 8A depicts tumor IL-12 levels in mice that received Ad-RTS-mIL-12+veledimex+anti-PD-1, Ad-RTS-mIL-12+veledimex monotherapy, or anti-PD-1 monotherapy.

FIG. 8B depicts tumor IFN-γ levels in mice received Ad-RTS-mIL-12+veledimex+anti-PD-1, Ad-RTS-mIL-12+veledimex monotherapy, or anti-PD-1 monotherapy.

FIG. 9A depicts effects on cytotoxic T cells (CD3⁺CD8⁺) in mice received Ad-RTS-mIL-12+veledimex+anti-PD-1, Ad-RTS-mIL-12+veledimex monotherapy, or anti-PD-1 monotherapy.

FIG. 9B depicts effects on T-cell exhaustion (Lag 3; CD233) in mice received Ad-RTS-mIL-12+veledimex+anti-PD-1, Ad-RTS-mIL-12+veledimex monotherapy, or anti-PD-1 monotherapy.

FIG. 10A depicts effects on Tregs (CD4⁺CD25⁺FoxP3⁺) in mice received Ad-RTS-mIL-12+veledimex+anti-PD-1, Ad-RTS-mIL-12+veledimex monotherapy, or anti-PD-1 monotherapy.

FIG. 10B depicts effects on ratio of cytotoxic T cells to regulator T cells (Treg) in mice that received Ad-RTS-mIL-12+veledimex+anti-PD-1, Ad-RTS-mIL-12+veledimex monotherapy, or anti-PD-1 monotherapy.

FIG. 11 is a schematic representation of the dosing regimen of the Ad-RTS-hIL-12+veledimex+anti-PD-1 antibody therapy in Example 5.

FIGS. 12A and 12B are a schematic representations of dosing regimens for Ad-RTS-hIL-12 plus veledimex therapy as described in Example 8 (“Main Study” (FIG. 12A) and “Expansion Substudy” (FIG. 12B)).

FIG. 13 shows the impact of dexamethasone use on overall survival in glioblastoma patients having received none-to-low dose dexamethasone (≤20 mg Dex) therapy (LoDex) as compared with higher-dose dexamethasone (>20 mg Dex) therapy (e.g., for medical management of post-operative edema).

FIGS. 14A and 14B show differences in serum cytokines (IL-12 (FIG. 12A) and Interferon-gamma (IFN-γ) (FIG. 12B)) in the Expansion Substudy, as compared with the Main Study (Days 0 through 28). Together, this shows sustained intratumoral production of cytokines in serum from recurrent glioblastoma patients.

FIGS. 15A and 15B show immune cell subtypes (CD3⁺CD8⁺ (cytotoxic T cells) (FIG. 15A); CD3⁺CD4⁺CD25^{hi}FOXP3⁺CD127^{lo/-} (regulatory T cells, Treg) (FIG. 15B) in the Expansion Substudy, as compared with the Main Study (Days 0 through 28). Together this shows immune cell infiltrates in whole blood from recurrent glioblastoma patients.

FIGS. 16A and 16B show “cytoindex” ratios of cytotoxic Tcells/Tregs as measured by the CD3⁺CD8⁺/CD3⁺CD4⁺CD25^{hi}FOXP3⁺CD127^{lo/-} immune cell ratio using flow cytometry. (A) An increase in cytoindex, which indicates an enhanced peripheral cytotoxic immune response, improved from 28 to a peak of 49 between Day 0 and 14 in the Expansion Substudy, as compared with the Main Study from 42 at a Day 0 to a peak of 74. (B) Replotting as a box plot combining the data from the Main Study and Expansion Substudy to increase sample size/power, the mean cytoindex (shaded arrows) increased from Day 0 to Day 7 to Day 14 before decreasing by Day 28.

FIG. 17 shows schematic representation of dosing regimens for Ad-RTS-hIL-12 plus veledimex in combination with a PD-1 inhibitor (nivolumab) therapy as described in Example 10.

FIGS. 18A and 18B show serum cytokine levels for IL-12 (FIG. 18A) and Interferon-gamma (IFN-γ) (FIG. 18B) in 10 mg veledimex+nivolumab treatment groups as described in Example 10.

FIGS. 19A, 19B, and 19C show peripheral blood flow cytometry results for various immune cell types/markers (CD3⁺CD8⁺ (cytotoxic Tcells); CD3⁺CD4⁺CD25hi FOXP3⁺CD127lo/− (Treg cells)) as detected in 10 mg veledimex+nivolumab treatment groups as described in Example 10.

FIG. 20 is a Study Consort Diagram. Patient accrual at four institutions shown number of patients treated at doses of 10, 20, 30 and 40 mg of veledimex. The percent compliance represents the number of days that the oral veledimex was orally administered vs. the 14-day total that each patient was expected to take the drug.

FIGS. 21A-21D show a series of graphs depicting veledimex, IL-12 and IFN-γ concentrations upon treatment with veledimex. FIG. 21A shows Peak plasma concentrations of veledimex at each drug dosage. Each symbol represents plasma from a single patient. FIG. 21B shows veledimex in plasma and intumorally at the time of surgical resection. Veledimex was administered to each patient approximately 3 hours before the start of the craniotomy. Serum and tumor at the time of resection were then assayed for veledimex levels. FIG. 21C shows Interleukin-12 in peripheral blood before, during, and after veledimex dosing. FIG. 21D shows Interferon-γ in peripheral blood before, during and after veledimex dosing. * denotes P<0.05.

FIGS. 22A-22D show radiologic and immunologic analyses of post-treatment tumors. Three patients with suspected progression post-treatment underwent re-resection of contrast-enhancing suspected tumor. FIG. 22A shows MRI images from one patient who had a right occipital recurrent GBM resected. The MRI scan, one day after surgery (Baseline), and then at weeks 4, 8 and 24 are shown. The injections were given in an area of the occipital lobe and one area more superior towards the parietal lobe. Red and Yellow arrows show areas with changes in enhancement in the occipital and parietal needle tracks. FIG. 22B shows images of GBM from the same patient shown in FIG. 22A. Left panels: GBM from one of the same patients shown in FIG. 22A at the time of resection before injection of Ad-RTS-hIL-12 (shown in the top panel at 20× magnification [Scale bar 100 μm] and in the bottom panel at 100× magnification [Scale bar 50 μm]). Right panels: GBM from the same patient 175 days after treatment (at time of suspected pseudoprogression). Resected material from the occipital lesion was analyzed by immunofluorescent histochemistry for expression of CD3+(yellow), CD8+(red), CD3+CD8+(orange), PD-1+(green), PD-L1+(cyan) and GFAP (white) (shown in the top panel at 20× magnification [Scale bar 100 μm] and in the bottom panel at 100× magnification [Scale bar 50 μm). FIGS. 22C and 22D show quantitative analyses of Pre- (Baseline) and Post-treatment expression of immunologic markers in tumor for the 3 patients undergoing re-resection after injection. FIG. 22C shows counts of CD3+, CD3+CD8+, PD-1+, CD3+CD4+FoxP3+, CD56+, and PD-L1+ expressing cells per mm2 of tumor. FIG. 22D shows IFN-γ in the 3 GBMs before and after treatment.

FIGS. 23A and 23B show a survival curve and analysis of treatment efficacy. FIG. 23A shows Kaplan-Meier overall survival for the 20 mg vs. combined 10, 30 and 40 mg cohorts. FIG. 23B shows a Survival Swimline. The x-axis lists survival time in months, with each patient number on the y-axis. Blue and green colors represent patients who were administered with less than or equal to 20 mg, or with greater than 20 mg of cumulative dexamethasone during days 0-14 of veledimex treatment. The 10, 20, 30, 40 mg veledimex designations at end of each bar represent the dose of veledimex each patient received. Patients on steroids at entry, times of progressive disease, and other therapy timelines are listed. The median OS was 12.7 months.

FIG. 24 shows a forest plot of prognostic factors of subgroups examined for overall survival.

FIGS. 25A-25C show graphs of survival probability for subjects. FIG. 25A shows a Kaplan-Meier survival based on cumulative dexamethasone dosage (less than or equal to 20 mg dexamethasone, or greater than 20 mg dexamethasone) for subjects. FIG. 25A shows the survival probability of subjects administered with 10, 20, 30, or 40 mg of veledimex (Days 0-14). FIG. 25B shows the survival probability of subjects administered with 20 mg of veledimex (Days 0-14). FIG. 25C shows the peripheral blood CD8+/FoxP3 ratio at 14 to 28 days after viral injection. Triangles represent deceased patients and squares represent alive patients.

FIG. 26 shows histo- and immuno-pathological features of tumor, pre and post Ad-RTS-hIL-12+VDX treatment. Five subjects underwent re-resection for suspected progression after gene therapy injection and VDX treatment. These 5 subjects (PT10, PT17, PT37, PT38, and PT39 were all diagnosed as Glioblastoma, IDH-wildtype, W.H.O. grade IV). Post-mortem tissue was available for PT37. Tumor tissue at the time of virus injection (Day 0) and post-injection (day 31 for PT10; day 45 for PT17; day 130 for PT38; day 159 for PT39 and day 176 for PT37 as well as postmortem tumor at day 505) were evaluated by standard Hematoxylin & Eosin (H & E) staining and by CD8 immunohistochemistry. For PT37, tumor from a resection that had occurred at first diagnosis (day −230) is also included. In all cases, a mild to marked increase in CD8+ cytotoxic T cells was observed surrounding blood vessels and infiltrating tumor. For PT37, post-mortem examination at 505 days showed minimal CD8+ cells, in contrast to the marked increase observed at 176 days post virus injection. All images were taken with 20× objective. Additional neuropathologic and genetic features were as follows: PT37: Tumor noted to lose amplifications of PIK3C2B/MDM4 and AKT3 between initial diagnosis/treatment and virus injection. Subsequent tumors had prominent giant cell morphology. Autopsy contained classic glioblastoma morphology as well as giant cell and myxoid areas. PT38: Post treatment tumor (130 days) contained prominent spindle cell morphology. PT39: Tumor contained prominent giant cell morphology and was noted to have lost EGFR and PDGFRA amplifications between virus injection and post-treatment tissue (159 days).

FIG. 27 shows a schematic diagram illustrating how Ad-RTS-hIL12+veledimex therapy drives downstream production IFN-γ and other cytokines via a cascade that elicits a brisk cytotoxic immune response.

### DETAILED DESCRIPTION

The methods provided herein use an adenovirus vector encoding an inducible RheoSwitch® Therapeutic System (RTS®) controlled human interleukin-12 (hIL-12), referred to herein as Ad-RTS-hIL-12. Transcription of the RTS-hIL-12 transgene only occurs in the presence of the diacylhydrazine activator ligand, veledimex. Human interleukin-12 (IL-12), a heterodimeric cytokine that enhances natural and adaptive immunity, potently stimulates production of interferon-γ (IFN-γ), and changes the composition of T-cells in the tumor microenvironment from Th0 to Th1 and CD8-positive T lymphocytes. Control of hIL-12 expression using the Ad-RTS-hIL-12 and veledimex system in the tumor microenviroment can be exploited to cause an increased influx of IFN-γ-producing CD8-positive T cells targeting the tumor. As overexpression of PD1 markers in the tumor microenvironment is elicited, the use of a PD-1 inhibitor can be used to improve treatment of the tumor. The methods provided herein use an anti-PD1 monoclonal antibody (mAb) checkpoint inhibitor, Nivolumab, in combination with Ad-RTS-hIL-12 and veledimex.

The methods provided herein are useful in treatment of cancer. Cancers amenable to treatment using the methods of the disclosure include for example, tumors of the central nervous system, malignant gliomas, primary glioblastoma, recurrent glioblastoma, progressive glioblastoma, or diffuse intrinsic pontine glioma (DIPG) and diffuse midline glioma tumors (e.g., in the thalamus, brainstem or spinal cord). The methods provided herein are useful for treatment of adult and pediatric patients.

### Adenoviral Vector

Suitable viral vectors used in the invention include, but not limited to, adenovirus-based vectors. Adenovirus (Ad) is a 36 kb double-stranded DNA virus that efficiently transfers DNA in vivo to a variety of different target cell types. The adenoviral vector can be produced in high titers and can efficiently transfer DNA to replicating and non-replicating cells. The adenoviral vector genome can be generated using any species, strain, subtype, mixture of species, strains, or subtypes, or chimeric adenovirus as the source of vector DNA. Adenoviral stocks that can be employed as a source of adenovirus can be amplified from the adenoviral serotypes 1 through 51, which are currently available from the American Type Culture Collection (ATCC, Manassas, Va.), or from any other serotype of adenovirus available from any other source. For instance, an adenovirus can be of subgroup A (e.g., serotypes 12, 18, and 31), subgroup B (e.g., serotypes 3, 7, 11, 14, 16, 21, 34, and 35), subgroup C (e.g., serotypes 1, 2, 5, and 6), subgroup D (e.g., serotypes 8, 9, 10, 13, 15, 17, 19, 20, 22-30, 32, 33, 36-39, and 42-47), subgroup E (serotype 4), subgroup F (serotypes 40 and 41), or any other adenoviral serotype. Given that the human adenovirus serotype 5 (Ad5) genome has been completely sequenced, the adenoviral vector of the invention is described herein with respect to the Ad5 serotype. The adenoviral vector can be any adenoviral vector capable of growth in a cell, which is in some significant part (although not necessarily substantially) derived from or based upon the genome of an adenovirus. The adenoviral vector can be based on the genome of any suitable wild-type adenovirus. In certain embodiments, the adenoviral vector is derived from the genome of a wild-type adenovirus of group C, especially of serotype 2 or 5. Adenoviral vectors are well known in the art and are described in, for example, U.S. Pat. Nos. 5,559,099, 5,712,136, 5,731,190, 5,837,511, 5,846,782, 5,851,806, 5,962,311, 5,965,541, 5,981,225, 5,994,106, 6,020,191, and 6,113,913, International Patent Applications WO 95/34671, WO 97/21826, and WO 00/00628, and Thomas Shenk, “Adenoviridae and their Replication,” and M. S. Horwitz, “Adenoviruses,” Chapters 67 and 68, respectively, in Virology, B. N. Fields et al., eds., 3d ed., Raven Press, Ltd., New York (1996).

In other embodiments, the adenoviral vector is replication-deficient. The term “replication-deficient” used herein means that the adenoviral vector comprises a genome that lacks at least one replication-essential gene function. A deficiency in a gene, gene function, or gene or genomic region, as used herein, is defined as a deletion of sufficient genetic material of the viral genome to impair or obliterate the function of the gene whose nucleic acid sequence was deleted in whole or in part. Replication-essential gene functions are those gene functions that are required for replication (i.e., propagation) of a replication-deficient adenoviral vector. Replication-essential gene functions are encoded by, for example, the adenoviral early regions (e.g., the E1, E2, and E4 regions), late regions (e.g., the L1-L5 regions), genes involved in viral packaging (e.g., the IVa2 gene), and virus-associated RNAs (e.g., VA-RNA I and/or VA-RNA II). In still other embodiments, the replication-deficient adenoviral vector comprises an adenoviral genome deficient in at least one replication-essential gene function of one or more regions of an adenoviral genome (e.g., two or more regions of an adenoviral genome to result in a multiply replication-deficient adenoviral vector). The one or more regions of the adenoviral genome are selected from the group consisting of the E1, E2, and E4 regions. The replication-deficient adenoviral vector can comprise a deficiency in at least one replication-essential gene function of the E1 region (denoted an E1-deficient adenoviral vector), particularly a deficiency in a replication-essential gene function of each of the adenoviral E1A region and the adenoviral E1B region. In addition to such a deficiency in the E1 region, the recombinant adenovirus also can have a mutation in the major late promoter (MLP), as discussed in International Patent Application WO 00/00628. In a particular embodiment, the vector is deficient in at least one replication-essential gene function of the E1 region and at least part of the nonessential E3 region (e.g., an Xba I deletion of the E3 region) (denoted an E1/E3-deficient adenoviral vector).

In certain embodiments, the adenoviral vector is “multiply-deficient,” meaning that the adenoviral vector is deficient in one or more gene functions required for viral replication in each of two or more regions of the adenoviral genome. For example, the aforementioned E1-deficient or E1/E3-deficient adenoviral vector can be further deficient in at least one replication-essential gene function of the E4 region (denoted an E1/E4-deficient adenoviral vector). An adenoviral vector deleted of the entire E4 region can elicit a lower host immune response.

Alternatively, the adenoviral vector lacks replication-essential gene functions in all or part of the E1 region and all or part of the E2 region (denoted an E1/E2-deficient adenoviral vector). Adenoviral vectors lacking replication-essential gene functions in all or part of the E1 region, all or part of the E2 region, and all or part of the E3 region also are contemplated herein. If the adenoviral vector of the invention is deficient in a replication-essential gene function of the E2A region, the vector does not comprise a complete deletion of the E2A region, which is less than about 230 base pairs in length. Generally, the E2A region of the adenovirus codes for a DBP (DNA binding protein), a polypeptide required for DNA replication. DBP is composed of 473 to 529 amino acids depending on the viral serotype. It is believed that DBP is an asymmetric protein that exists as a prolate ellipsoid consisting of a globular Ct with an extended Nt domain. Studies indicate that the Ct domain is responsible for DBP's ability to bind to nucleic acids, bind to zinc, and function in DNA synthesis at the level of DNA chain elongation. However, the Nt domain is believed to function in late gene expression at both transcriptional and post-transcriptional levels, is responsible for efficient nuclear localization of the protein, and also may be involved in enhancement of its own expression. Deletions in the Nt domain between amino acids 2 to 38 have indicated that this region is important for DBP function (Brough et al., Virology, 196, 269-281 (1993)). While deletions in the E2A region coding for the Ct region of the DBP have no effect on viral replication, deletions in the E2A region which code for amino acids 2 to 38 of the Nt domain of the DBP impair viral replication. In one embodiment, the multiply replication-deficient adenoviral vector contains this portion of the E2A region of the adenoviral genome. In particular, for example, the desired portion of the E2A region to be retained is that portion of the E2A region of the adenoviral genome which is defined by the 5′ end of the E2A region, specifically positions Ad5(23816) to Ad5(24032) of the E2A region of the adenoviral genome of serotype Ad5.

The adenoviral vector can be deficient in replication-essential gene functions of only the early regions of the adenoviral genome, only the late regions of the adenoviral genome, and both the early and late regions of the adenoviral genome. The adenoviral vector also can have essentially the entire adenoviral genome removed, in which case at least either the viral inverted terminal repeats (ITRs) and one or more promoters or the viral ITRs and a packaging signal are left intact (i.e., an adenoviral amplicon). The larger the region of the adenoviral genome that is removed, the larger the piece of exogenous nucleic acid sequence that can be inserted into the genome. For example, given that the adenoviral genome is 36 kb, by leaving the viral ITRs and one or more promoters intact, the exogenous insert capacity of the adenovirus is approximately 35 kb. Alternatively, a multiply deficient adenoviral vector that contains only an ITR and a packaging signal effectively allows insertion of an exogenous nucleic acid sequence of approximately 37-38 kb. Of course, the inclusion of a spacer element in any or all of the deficient adenoviral regions will decrease the capacity of the adenoviral vector for large inserts. Suitable replication-deficient adenoviral vectors, including multiply deficient adenoviral vectors, are disclosed in U.S. Pat. Nos. 5,851,806 and 5,994,106 and International Patent Applications WO 95/34671 and WO 97/21826. In one embodiment, the vector for use in the present inventive method is that described in International Patent Application PCT/US01/20536.

It should be appreciated that the deletion of different regions of the adenoviral vector can alter the immune response of the mammal. In particular, the deletion of different regions can reduce the inflammatory response generated by the adenoviral vector. Furthermore, the adenoviral vector's coat protein can be modified to decrease the adenoviral vector's ability or inability to be recognized by a neutralizing antibody directed against the wild-type coat protein, as described in International Patent Application WO 98/40509.

The adenoviral vector, when multiply replication-deficient, especially in replication-essential gene functions of the E1 and E4 regions, can include a spacer element to provide viral growth in a complementing cell line similar to that achieved by singly replication deficient adenoviral vectors, particularly an adenoviral vector comprising a deficiency in the E1 region. The spacer element can contain any sequence or sequences which are of the desired length. The spacer element sequence can be coding or non-coding and native or non-native with respect to the adenoviral genome, but it does not restore the replication-essential function to the deficient region. In the absence of a spacer, production of fiber protein and/or viral growth of the multiply replication-deficient adenoviral vector is reduced by comparison to that of a singly replication-deficient adenoviral vector. However, inclusion of the spacer in at least one of the deficient adenoviral regions, preferably the E4 region, can counteract this decrease in fiber protein production and viral growth. The use of a spacer in an adenoviral vector is described in U.S. Pat. No. 5,851,806.

Construction of adenoviral vectors is well understood in the art. Adenoviral vectors can be constructed and/or purified using the methods set forth, for example, in U.S. Pat. No. 5,965,358 and International Patent Applications WO 98/56937, WO 99/15686, and WO 99/54441. The production of adenoviral gene transfer vectors is well known in the art, and involves using standard molecular biological techniques such as those described in, for example, Sambrook et al., supra, Watson et al., supra, Ausubel et al., supra, and in several of the other references mentioned herein.

Replication-deficient adenoviral vectors are typically produced in complementing cell lines that provide gene functions not present in the replication-deficient adenoviral vectors, but required for viral propagation, at appropriate levels in order to generate high titers of viral vector stock. In one embodiment, a cell line complements for at least one and/or all replication-essential gene functions not present in a replication-deficient adenovirus. The complementing cell line can complement for a deficiency in at least one replication-essential gene function encoded by the early regions, late regions, viral packaging regions, virus-associated RNA regions, or combinations thereof, including all adenoviral functions (e.g., to enable propagation of adenoviral amplicons, which comprise minimal adenoviral sequences, such as only inverted terminal repeats (ITRs) and the packaging signal or only ITRs and an adenoviral promoter). In another embodiment, the complementing cell line complements for a deficiency in at least one replication-essential gene function (e.g., two or more replication-essential gene functions) of the E1 region of the adenoviral genome, particularly a deficiency in a replication-essential gene function of each of the E1A and E1B regions. In addition, the complementing cell line can complement for a deficiency in at least one replication-essential gene function of the E2 (particularly as concerns the adenoviral DNA polymerase and terminal protein) and/or E4 regions of the adenoviral genome. Desirably, a cell that complements for a deficiency in the E4 region comprises the E4-ORF6 gene sequence and produces the E4-ORF6 protein. Such a cell desirably comprises at least ORF6 and no other ORF of the E4 region of the adenoviral genome. The cell line preferably is further characterized in that it contains the complementing genes in a non-overlapping fashion with the adenoviral vector, which minimizes, and practically eliminates, the possibility of the vector genome recombining with the cellular DNA. Accordingly, the presence of replication competent adenoviruses (RCA) is minimized if not avoided in the vector stock, which, therefore, is suitable for certain therapeutic purposes, especially gene therapy purposes. The lack of RCA in the vector stock avoids the replication of the adenoviral vector in non-complementing cells. The construction of complementing cell lines involves standard molecular biology and cell culture techniques, such as those described by Sambrook et al., supra, and Ausubel et al., supra. Complementing cell lines for producing the gene transfer vector (e.g., adenoviral vector) include, but are not limited to, 293 cells (described in, e.g., Graham et al., J. Gen. Virol., 36, 59-72 1977), PER.C6 cells (described in, e.g., International Patent Application WO 97/00326, and U.S. Pat. Nos. 5,994,128 and 6,033,908), and 293-ORF6 cells (described in, e.g., International Patent Application WO 95/34671 and Brough et al., J Virol., 71, 9206-9213 1997). The insertion of a nucleic acid sequence into the adenoviral genome (e.g., the E1 region of the adenoviral genome) can be facilitated by known methods, for example, by the introduction of a unique restriction site at a given position of the adenoviral genome.

The polynucleotide sequence in the expression vector is operatively linked to appropriate expression control sequence(s) including, for instance, a promoter to direct mRNA transcription. Representatives of additional promoters include, but are not limited to, constitutive promoters and tissue specific or inducible promoters. Examples of constitutive eukaryotic promoters include, but are not limited to, the promoter of the mouse metallothionein I gene (Hamer et al., J. Mol. Appl. Gen. 1:273 1982); the TK promoter of Herpes virus (McKnight, Cell 31:355 1982); the SV40 early promoter (Benoist et al., Nature 290:304 1981); and the vaccinia virus promoter. Additional examples of the promoters that could be used to drive expression of a protein or polynucleotide include, but are not limited to, tissue-specific promoters and other endogenous promoters for specific proteins, such as the albumin promoter (hepatocytes), a proinsulin promoter (pancreatic beta cells) and the like. In general, expression constructs will contain sites for transcription, initiation and termination and, in the transcribed region, a ribosome binding site for translation. The coding portion of the mature transcripts expressed by the constructs may include a translation initiating AUG at the beginning and a termination codon (UAA, UGA or UAG) appropriately positioned at the end of the polypeptide to be translated.

### Gene Switch Systems

The gene switch may be any gene switch that regulates gene expression by addition or removal of a specific ligand. In one embodiment, the gene switch is one in which the level of gene expression is dependent on the level of ligand that is present. Examples of ligand-dependent transcription factor complexes that may be used in the gene switches of the invention include, without limitation, members of the nuclear receptor superfamily activated by their respective ligands (e.g., glucocorticoid, estrogen, progestin, retinoid, ecdysone, and analogs and mimetics thereof) and rTTA activated by tetracycline. In one aspect of the invention, the gene switch is an EcR-based gene switch. Examples of such systems include, without limitation, the systems described in U.S. Pat. Nos. 6,258,603, 7,045,315, U.S. Published Patent Application Nos. 2006/001471 1, 2007/0161086, and International Published Application No. WO 01/70816. Examples of chimeric ecdysone receptor systems are described in U.S. Pat. No. 7,091,038, U.S. Published Patent Application Nos. 2002/0110861, 2004/0033600, 2004/0096942, 2005/0266457, and 2006/0100416, and International Published Application Nos. WO 01/70816, WO 02/066612, WO 02/066613, WO 02/066614, WO 02/066615, WO 02/29075, and WO 2005/108617, each of which is incorporated by reference in its entirety.

In another aspect of the invention, the gene switch is based on heterodimerization of FK506 binding protein (FKBP) with FKBP rapamycin associated protein (FRAP) and is regulated through rapamycin or its non-immunosuppressive analogs. Examples of such systems include, without limitation, the ARGENT™ Transcriptional Technology (ARIAD Pharmaceuticals, Cambridge, Mass.) and the systems described in U.S. Pat. Nos. 6,015,709, 6,117,680, 6,479,653, 6,187,757, and 6,649,595.

In one embodiment, the gene switch comprises a single transcription factor sequence encoding a ligand-dependent transcription factor complex under the control of a therapeutic switch promoter. The transcription factor sequence may encode a ligand-dependent transcription factor complex that is a naturally occurring or an artificial ligand-dependent transcription factor complex. An artificial transcription factor is one in which the natural sequence of the transcription factor has been altered, e.g., by mutation of the sequence or by the combining of domains from different transcription factors. In one embodiment, the transcription factor comprises a Group H nuclear receptor ligand binding domain. In one embodiment, the Group H nuclear receptor ligand binding domain is from an ecdysone receptor, a ubiquitous receptor (UR), an orphan receptor 1 (OR-1), a steroid hormone nuclear receptor 1 (NER-1), a retinoid X receptor interacting protein-15 (RIP-15), a liver X receptor β (LXRβ), a steroid hormone receptor like protein (RLD-1), a liver X receptor (LXR), a liver X receptor α (LXRα), a farnesoid X receptor (FXR), a receptor interacting protein 14 (RIP-14), or a farnesol receptor (HRR-1). In another embodiment, the Group H nuclear receptor LBD is from an ecdysone receptor.

The EcR and the other Group H nuclear receptors are members of the nuclear receptor superfamily wherein all members are generally characterized by the presence of an amino-terminal transactivation domain (AD, also referred to interchangeably as “TA” or “TD”), optionally fused to a heterodimerization partner (HP) to form a coactivation protein (CAP), a DNA binding domain (DBD), and an LBD fused to the DBD via a hinge region to form a ligand-dependent transcription factor (LTF). As used herein, the term “DNA binding domain” comprises a minimal polypeptide sequence of a DNA binding protein, up to the entire length of a DNA binding protein, so long as the DNA binding domain functions to associate with a particular response element. Members of the nuclear receptor superfamily are also characterized by the presence of four or five domains: A/B, C, D, E, and in some members F (see U.S. Pat. No. 4,981,784 and Evans, Science 240:889 (1988)). The “A/B” domain corresponds to the transactivation domain, “C” corresponds to the DNA binding domain, “D” corresponds to the hinge region, and “E” corresponds to the ligand binding domain. Some members of the family may also have another transactivation domain on the carboxy-terminal side of the LBD corresponding to “F”.

The following polypeptide sequence (Ecdysone receptor (878aa) from Drosophila melanogaster (Fruit fly) (SEQ ID NO: 9) is one example of a polypeptide sequence from an Ecdysone receptor (Ecdysteroid receptor) (20-hydroxy-ecdysone receptor) (20E receptor) (EcRH) (Nuclear receptor subfamily 1 group H member 1) and has the accession number P34021 in the GenBank database.

| (SEQ ID NO: 9) | |
|---|---|
| 1 | mkrrwsnngg fmrlpeesss evtsssnglv lpsgvnmsps sldshdycdq dlwlcgnesg |
| 61 | sfggsnghgl sqqqqsvitl amhgcsstlp aqttiiping nangnggstn gqyvpgatnl |
| 121 | galangmlng gfngmqqqiq nghglinstt pstpttplhl qqnlggaggg giggmgilhh |
| 181 | angtpnglig vvgggggvgl gvggggvggl gmqhtprsds vnsissgrdd lspssslngy |
| 241 | sanescdakk skkgpaprvq eelclvcgdr asgyhynalt cegckgffrr svtksavycc |
| 301 | kfgracemdm ymrrkcqecr lkkclavgmr pecvvpenqc amkrrekkaq kekdkmttsp |
| 361 | ssqhggngsl asgggqdfvk keildlmtce ppqhatipll pdeilakcqa rnipsltynq |
| 421 | laviykliwy qdgyeqpsee dlrrimsqpd enesqtdvsf rhiteitilt vqlivefakg |
| 481 | lpaftkipqe dqitllkacs sevmmlrmar rydhssdsif fannrsytrd sykmagmadn |
| 541 | iedllhfcrq mfsmkvdnve yalltaivif sdrpglekaq lveaiqsyyi dtlriyilnr |
| 601 | hcgdsmslvf yakllsilte lrtlgnqnae mcfslklknr klpkfleeiw dvhaippsvq |
| 661 | shlqitqeen erleraermr asvggaitag idcdsastsa aaaaaqhqpq pqpqpqpssl |
| 721 | tqndsqhqtq pqlqpqlppq lqgqlqpqlq pqlqtqlqpq iqpqpqllpv sapvpasvta |
| 781 | pgslsavsts seymggsaai gpitpattss itaavtasst tsavpmgngv gvgvgvggnv |
| 841 | smyanaqtam almgvalhsh qeqliggvav ksehstta |

The DBD is characterized by the presence of two cysteine zinc fingers between which are two amino acid motifs, the P-box and the D-box, which confer specificity for response elements. These domains may be either native, modified, or chimeras of different domains of heterologous receptor proteins. The EcR, like a subset of the nuclear receptor family, also possesses less well-defined regions responsible for heterodimerization properties. Because the domains of nuclear receptors are modular in nature, the LBD, DBD, and AD may be interchanged.

In another embodiment, the transcription factor comprises an AD, a DBD that recognizes a response element associated with the therapeutic protein or therapeutic polynucleotide whose expression is to be modulated; and a Group H nuclear receptor LBD. In certain embodiments, the Group H nuclear receptor LBD comprises a substitution mutation.

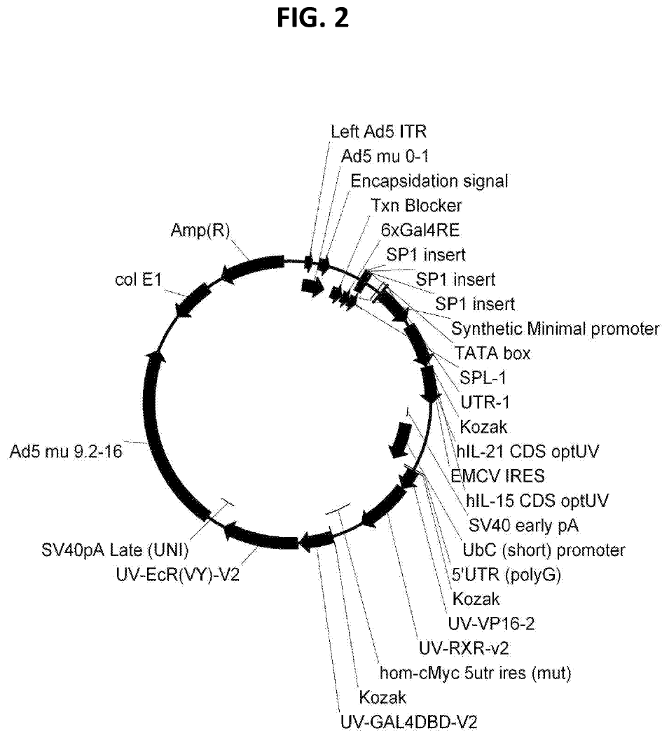

In another embodiment, the gene switch comprises a first transcription factor sequence, e.g., a CAP, under the control of a first therapeutic switch promoter (TSP-1) and a second transcription factor sequence, e.g., a LTF, under the control of a second therapeutic switch promoter (TSP-2), wherein the proteins encoded by said first transcription factor sequence and said second transcription factor sequence interact to form a protein complex (LDTFC), i.e., a “dual switch”- or “two-hybrid”-based gene switch. The first and second TSPs may be the same or different. In this embodiment, the presence of two different TSPs in the gene switch that are required for therapeutic molecule expression enhances the specificity of the therapeutic method (see FIG. 2). FIG. 2 also demonstrates the ability to modify the therapeutic gene switch to treat any disease, disorder, or condition simply by inserting the appropriate TSPs.

In a further embodiment, both the first and the second transcription factor sequence, e.g., a CAP or an LTF, are under the control of a single therapeutic switch promoter (e.g., TSP-1). Activation of this promoter will generate both CAP and LTF with a single open reading frame. This can be achieved with the use of a transcriptional linker such as an IRES (internal ribosomal entry site). In this embodiment, both portions of the ligand-dependent transcription factor complex are synthesized upon activation of TSP-1. TSP-1 can be a constitutive promoter or only activated under conditions associated with the disease, disorder, or condition.

In a further embodiment, one transcription factor sequence, e.g. a LTF, is under the control of a therapeutic switch promoter only activated under conditions associated with the disease, disorder, or condition (e.g., TSP-2 or TSP-3) and the other transcription factor sequence, e.g., CAP, is under the control of a constitutive therapeutic switch promoter (e.g., TSP-1). In this embodiment, one portion of the ligand-dependent transcription factor complex is constitutively present while the second portion will only be synthesized under conditions associated with the disease, disorder, or condition.

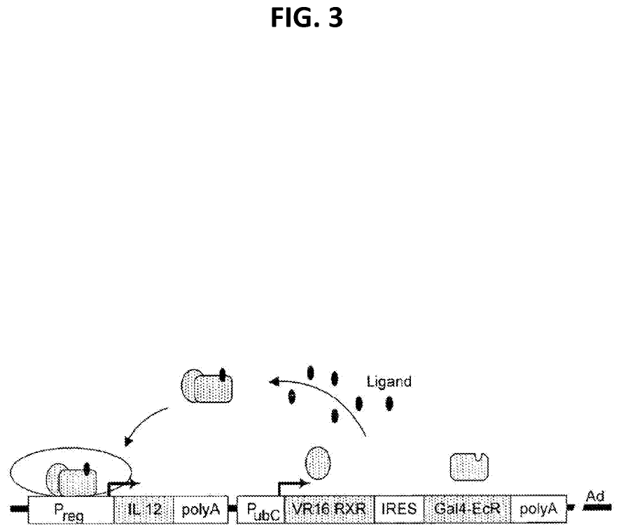

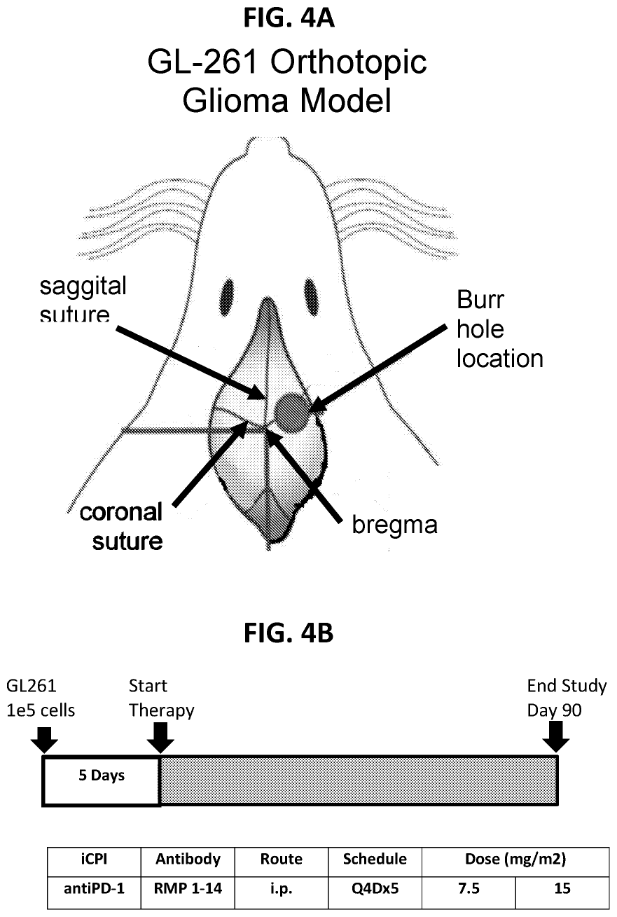

In another embodiment, one transcription factor sequence, e.g., CAP, is under the control of a first TSP (e.g., TSP-1) and two or more different second transcription factor sequences, e.g., LTF-1 and LTF-2 are under the control of different TSPs (e.g., TSP-2 and TSP-3 in FIG. 3). In this embodiment, each of the LTFs may have a different DBD that recognizes a different factor-regulated promoter sequence (e.g., DBD-A binds to a response element associated with factor-regulated promoter-1 (FRP-1) and DBD-B binds to a response element associated with factor-regulated promoter-2 (FRP-2). Each of the factor-regulated promoters may be operably linked to a different therapeutic gene. In this manner, multiple treatments may be provided simultaneously.

In one embodiment, the first transcription factor sequence encodes a polypeptide comprising a TAD (transactivation domain), a DBD (DNA binding domain) that recognizes a response element associated with the therapeutic product sequence whose expression is to be modulated; and a Group H nuclear receptor LBD (ligand binding domain), and the second transcription factor sequence encodes a transcription factor comprising a nuclear receptor LBD selected from a vertebrate retinoid X receptor (RXR), an invertebrate RXR, an ultraspiracle protein (USP), or a chimeric nuclear receptor comprising at least two different nuclear receptor ligand binding domain polypeptide fragments selected from a vertebrate RXR, an invertebrate RXR, and a USP (see WO 01/70816 A2 and US 2004/0096942 A1). The “partner” nuclear receptor ligand binding domain may further comprise a truncation mutation, a deletion mutation, a substitution mutation, or another modification.

In another embodiment, the gene switch comprises a first transcription factor sequence encoding a first polypeptide comprising a nuclear receptor LBD and a DBD that recognizes a response element associated with the therapeutic product sequence whose expression is to be modulated, and a second transcription factor sequence encoding a second polypeptide comprising an AD and a nuclear receptor LBD, wherein one of the nuclear receptor LBDs is a Group H nuclear receptor LBD. In one embodiment, the first polypeptide is substantially free of an AD and the second polypeptide is substantially free of a DBD. For purposes of the invention, “substantially free” means that the protein in question does not contain a sufficient sequence of the domain in question to provide activation or binding activity.

In another aspect of the invention, the first transcription factor sequence encodes a protein comprising a heterodimerization partner and an AD (a “CAP”) and the second transcription factor sequence encodes a protein comprising a DBD and an LBD (an “LTF”).

When only one nuclear receptor LBD is a Group H LBD, the other nuclear receptor LBD may be from any other nuclear receptor that forms a dimer with the Group H LBD. For example, when the Group H nuclear receptor LBD is an EcR LBD, the other nuclear receptor LBD “partner” may be from an EcR, a vertebrate RXR, an invertebrate RXR, an ultraspiracle protein (USP), or a chimeric nuclear receptor comprising at least two different nuclear receptor LBD polypeptide fragments selected from a vertebrate RXR, an invertebrate RXR, or a USP (see WO 01/70816 A2, International Patent Application No. PCT/US02/05235 and US 2004/0096942 A1, incorporated herein by reference in their entirety). The “partner” nuclear receptor ligand binding domain may further comprise a truncation mutation, a deletion mutation, a substitution mutation, or another modification.

In one embodiment, the vertebrate RXR LBD is from a human Homo sapiens, mouse Mus musculus, rat Rattus norvegicus, chicken Gallus gallus, pig Sus scrofa domestica, frog Xenopus laevis, zebrafish Danio rerio, tunicate Polyandrocarpa misakiensis, or jellyfish Tripedalia cysophora RXR.

In one embodiment, the invertebrate RXR ligand binding domain is from a locust Locusta migratoria ultraspiracle polypeptide (“LmUSP”), an ixodid tick Amblyomma americanum RXR homolog 1 (“AmaRXR1”), an ixodid tick Amblyomma americanum RXR homolog 2 (“AmaRXR2”), a fiddler crab Celuca pugilator RXR homolog (“CpRXR”), a beetle Tenebrio molitor RXR homolog (“TmRXR”), a honeybee Apis mellifera RXR homolog (“AmRXR”), an aphid Myzus persicae RXR homolog (“MpRXR”), or a non-Dipteran/non-Lepidopteran RXR homolog.

In one embodiment, the chimeric RXR LBD comprises at least two polypeptide fragments selected from a vertebrate species RXR polypeptide fragment, an invertebrate species RXR polypeptide fragment, or a non-Dipteran/non-Lepidopteran invertebrate species RXR homolog polypeptide fragment. A chimeric RXR ligand binding domain for use in the present invention may comprise at least two different species RXR polypeptide fragments, or when the species is the same, the two or more polypeptide fragments may be from two or more different isoforms of the species RXR polypeptide fragment. Such chimeric RXR LBDs are disclosed, for example, in WO 2002/066614.

In one embodiment, the chimeric RXR ligand binding domain comprises at least one vertebrate species RXR polypeptide fragment and one invertebrate species RXR polypeptide fragment.

In another embodiment, the chimeric RXR ligand binding domain comprises at least one vertebrate species RXR polypeptide fragment and one non-Dipteran/non-Lepidopteran invertebrate species RXR homolog polypeptide fragment.

The ligand, when combined with the LBD of the nuclear receptor(s), which in turn are bound to the response element of an FRP associated with a therapeutic product sequence, provides external temporal regulation of expression of the therapeutic product sequence. The binding mechanism or the order in which the various components of this invention bind to each other, that is, for example, ligand to LBD, DBD to response element, AD to promoter, etc., is not critical.

In a specific example, binding of the ligand to the LBD of a Group H nuclear receptor and its nuclear receptor LBD partner enables expression of the therapeutic product sequence. This mechanism does not exclude the potential for ligand binding to the Group H nuclear receptor (GHNR) or its partner, and the resulting formation of active homodimer complexes (e.g. GHNR+GHNR or partner+partner). Preferably, one or more of the receptor domains is varied producing a hybrid gene switch. Typically, one or more of the three domains, DBD, LBD, and AD, may be chosen from a source different than the source of the other domains so that the hybrid genes and the resulting hybrid proteins are optimized in the chosen host cell or organism for transactivating activity, complementary binding of the ligand, and recognition of a specific response element. In addition, the response element itself can be modified or substituted with response elements for other DNA binding protein domains such as the GAL-4 protein from yeast (see Sadowski et al., Nature 335:563 (1988)) or LexA protein from Escherichia coli (see Brent et al., Cell 43:729 (1985)), or synthetic response elements specific for targeted interactions with proteins designed, modified, and selected for such specific interactions (see, for example, Kim et al., Proc. Natl. Acad Sci. USA, 94:3616 (1997)) to accommodate hybrid receptors. Another advantage of two-hybrid systems is that they allow choice of a promoter used to drive the gene expression according to a desired end result. Such double control may be particularly important in areas of gene therapy, especially when cytotoxic proteins are produced, because both the timing of expression as well as the cells wherein expression occurs may be controlled. When genes, operably linked to a suitable promoter, are introduced into the cells of the subject, expression of the exogenous genes is controlled by the presence of the system of this invention. Promoters may be constitutively or inducibly regulated or may be tissue-specific (that is, expressed only in a particular cell type) or specific to certain developmental stages of the organism.

The DNA binding domain of the first hybrid protein binds, in the presence or absence of a ligand, to the DNA sequence of a response element to initiate or suppress transcription of downstream gene(s) under the regulation of this response element.

The functional LDTFC, e.g., an EcR complex, may also include additional protein(s) such as immunophilins. Additional members of the nuclear receptor family of proteins, known as transcriptional factors (such as DHR38 or betaFTZ-1), may also be ligand dependent or independent partners for EcR, USP, and/or RXR. Additionally, other cofactors may be required such as proteins generally known as coactivators (also termed adapters or mediators). These proteins do not bind sequence-specifically to DNA and are not involved in basal transcription. They may exert their effect on transcription activation through various mechanisms, including stimulation of DNA-binding of activators, by affecting chromatin structure, or by mediating activator-initiation complex interactions. Examples of such coactivators include RIP140, TIF1, RAP46/Bag-1, ARA70, SRC-1/NCoA-1, TIF2/GRIP/NCoA-2, ACTR/AIB1/RAC3/pCIP as well as the promiscuous coactivator C response element B binding protein, CBP/p300 (for review see Glass et al., Curr. Opin. Cell Biol. 9:222 (1997)). Also, protein cofactors generally known as corepressors (also known as repressors, silencers, or silencing mediators) may be required to effectively inhibit transcriptional activation in the absence of ligand. These corepressors may interact with the unliganded EcR to silence the activity at the response element. Current evidence suggests that the binding of ligand changes the conformation of the receptor, which results in release of the corepressor and recruitment of the above described coactivators, thereby abolishing their silencing activity. Examples of corepressors include N-CoR and SMRT (for review, see Horwitz et al., Mol Endocrinol. 10:1167 (1996)). These cofactors may either be endogenous within the cell or organism, or may be added exogenously as transgenes to be expressed in either a regulated or unregulated fashion.

In a preferred embodiment, an ecdysone receptor-based gene switch as may be used in the present invention is described in WO 2002/066612 (PCT/US2002/005090, filed Feb. 20, 2002, published Aug. 29, 2002) which is hereby incorporated by reference in its entirety.

In additional embodiments, ecdysone receptor-based gene switches that may be used in the present invention are described in WO 2001/070816 (PCT/US01/09050, filed Mar. 21, 2001, published Sep. 27, 2001); WO 2002/066614 (PCT/US02/05706, filed Feb. 20, 2002, published Aug. 29, 2002); and WO 2002/066615 (PCT/US02/05708, filed Feb. 20, 2002, published Aug. 29, 2002) each of which are hereby incorporated by reference in their entirety.

### Ligands

As used herein, the term “ligand,” as applied to ligand-activated ecdysone receptor-based gene switches are small molecules of varying solubility having the capability of activating a gene switch to stimulate expression of a polypeptide encoded therein. The ligand for a ligand-dependent transcription factor complex of the invention binds to the protein complex comprising one or more of the ligand binding domain, the heterodimer partner domain, the DNA binding domain, and the transactivation domain. The choice of ligand to activate the ligand-dependent transcription factor complex depends on the type of the gene switch utilized.

Examples of ligands include, without limitation, an ecdysteroid, such as ecdysone, 20-hydroxyecdysone, ponasterone A, muristerone A, and the like, 9-cis-retinoic acid, synthetic analogs of retinoic acid, N,N′-diacylhydrazines such as those disclosed in U.S. Pat. Nos. 6,013,836; 5,117,057; 5,530,028; and 5,378,726 and U.S. Published Application Nos. 2005/0209283 and 2006/0020146; oxadiazolines as described in U.S. Published Application No. 2004/0171651; dibenzoylalkyl cyanohydrazines such as those disclosed in European Application No. 461,809; N-alkyl-N,N′-diaroylhydrazines such as those disclosed in U.S. Pat. No. 5,225,443; N-acyl-N-alkylcarbonylhydrazines such as those disclosed in European Application No. 234,994; N-aroyl-N-alkyl-N′-aroylhydrazines such as those described in U.S. Pat. No. 4,985,461; amidoketones such as those described in U.S. Published Application No. 2004/0049037; each of which is incorporated herein by reference and other similar materials including 3,5-di-tert-butyl-4-hydroxy-N-isobutyl-benzamide, 8-O-acetylharpagide, oxysterols, 22(R) hydroxycholesterol, 24(S) hydroxycholesterol, 25-epoxycholesterol, T0901317, 5-alpha-6-alpha-epoxycholesterol-3-sulfate (ECHS), 7-ketocholesterol-3-sulfate, famesol, bile acids, 1,1-biphosphonate esters, juvenile hormone III, and the like. Examples of diacylhydrazine ligands useful in the present invention include RG-115819 (3,5-Dimethyl-benzoic acid N-(1-ethyl-2,2-dimethyl-propyl)-N′-(2-methyl-3-methoxybenzoyl)-hydrazide), RG-115932 ((R)-3,5-Dimethyl-benzoic acid N-(1-tert-butyl-butyl)N′-(2-ethyl-3-methoxy-benzoyl)-hydrazide), and RG-115830 (3,5-Dimethyl-benzoic acid N-(1-tert-butyl-butyl)-N′-(2-ethyl-3-methoxy-benzoyl)-hydrazide). See, e.g., U.S. patent application Ser. No. 12/155,111, and PCT Appl. No. PCT/US2008/006757, both of which are incorporated herein by reference in their entireties.

For example, a ligand for the ecdysone receptor-based gene switch may be selected from any suitable ligands. Both naturally occurring ecdysone or ecdysone analogs (e.g., 20-hydroxyecdysone, muristerone A, ponasterone A, ponasterone B, ponasterone C, 26-iodoponasterone A, inokosterone or 26-mesylinokosterone) and non-steroid inducers may be used as a ligand for gene switch of the present invention. U.S. Pat. No. 6,379,945 BI, describes an insect steroid receptor isolated from Heliothis virescens (“HEcR”) which is capable of acting as a gene switch responsive to both steroid and certain non-steroidal inducers. Non-steroidal inducers have a distinct advantage over steroids, in this and many other systems which are responsive to both steroids and non-steroid inducers, for several reasons including, for example: lower manufacturing cost, metabolic stability, absence from insects, plants, or mammals, and environmental acceptability. U.S. Pat. No. 6,379,945 B1 describes the utility of two dibenzoylhydrazines, 1,2-dibenzoyl-1-tert-butyl-hydrazine and tebufenozide (N-(4-ethylbenzoyl)-N′-(3,5-dimethylbenzoyl)-N′-tert-butyl-hydrazine) as ligands for an ecdysone-based gene switch. Also included in the present invention as a ligand are other dibenzoylhydrazines, such as those disclosed in U.S. Pat. No. 5,117,057 B1. Use of tebufenozide as a chemical ligand for the ecdysone receptor from Drosophila melanogaster is also disclosed in U.S. Pat. No. 6,147,282. Additional, non-limiting examples of ecdysone ligands are 3,5-di-tert-butyl-4-hydroxy-N-isobutyl-benzamide, 8-O-acetylharpagide, a 1,2-diacyl hydrazine, an N′-substituted-N,N′-disubstituted hydrazine, a dibenzoylalkyl cyanohydrazine, an N-substituted-N-alkyl-N,N-diaroyl hydrazine, an N-substituted-N-acyl-N-alkyl, carbonyl hydrazine or an N-aroyl-N′-alkylN′-aroyl hydrazine. (See U.S. Pat. No. 6,723,531).

In one embodiment, the ligand for an ecdysone-based gene switch system is a diacylhydrazine ligand or chiral diacylhydrazine ligand. The ligand used in the gene switch system may be compounds of Formula I

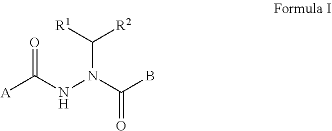

wherein A is alkoxy, arylalkyloxy or aryloxy; B is optionally substituted aryl or optionally substituted heteroaryl; and R1 and R2 are independently optionally substituted alkyl, arylalkyl, hydroxyalkyl, haloalkyl, optionally substituted cycloalkyl, optionally substituted alkenyl, optionally substituted alkynyl, optionally substituted heterocyclo, optionally substituted aryl or optionally substituted heteroaryl; or pharmaceutically acceptable salts, hydrates, crystalline forms or amorphous forms thereof.

In another embodiment, the ligand may be enantiomerically enriched compounds of Formula II

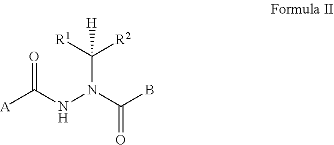

wherein A is alkoxy, arylalkyloxy, aryloxy, arylalkyl, optionally substituted aryl or optionally substituted heteroaryl; B is optionally substituted aryl or optionally substituted heteroaryl; and R1 and R2 are independently optionally substituted alkyl, arylalkyl, hydroxyalkyl, haloalkyl, optionally substituted cycloalkyl, optionally substituted alkenyl, optionally substituted alkynyl, optionally substituted heterocyclo, optionally substituted aryl or optionally substituted heteroaryl; with the proviso that R1 does not equal R2; wherein the absolute configuration at the asymmetric carbon atom bearing R1 and R2 is predominantly S; or pharmaceutically acceptable salts, hydrates, crystalline forms or amorphous forms thereof.

In certain embodiments, the ligand may be enantiomerically enriched compounds of Formula III

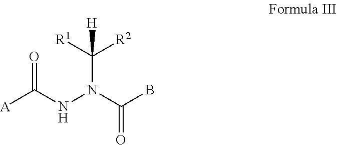

wherein A is alkoxy, arylalkyloxy, aryloxy, arylalkyl, optionally substituted aryl or optionally substituted heteroaryl; B is optionally substituted aryl or optionally substituted heteroaryl; and R1 and R2 are independently optionally substituted alkyl, arylalkyl, hydroxyalkyl, haloalkyl, optionally substituted cycloalkyl, optionally substituted alkenyl, optionally substituted alkynyl, optionally substituted heterocyclo, optionally substituted aryl or optionally substituted heteroaryl; with the proviso that R1 does not equal R2; wherein the absolute configuration at the asymmetric carbon atom bearing R1 and R2 is predominantly R; or pharmaceutically acceptable salts, hydrates, crystalline forms or amorphous forms thereof.

In one embodiment, a ligand may be (R)-3,5-dimethyl-benzoic acid N-(1-tertbutyl-butyl)-N′-(2-ethyl-3-methoxy-benzoyl)-hydrazide having an enantiomeric excess of at least 95% or a pharmaceutically acceptable salt, hydrate, crystalline form or amorphous form thereof.

The diacylhydrazine ligands of Formula I and chiral diacylhydrazine ligands of Formula II or III, when used with an ecdysone-based gene switch system, provide the means for external temporal regulation of expression of a therapeutic polypeptide or therapeutic polynucleotide of the present invention. See U.S. application Ser. No. 12/155,111, filed May 29, 2008, which is fully incorporated by reference herein.

The ligands used in the present invention may form salts. The term “salt(s)” as used herein denotes acidic and/or basic salts formed with inorganic and/or organic acids and bases. In addition, when a compound of Formula I, II or III contains both a basic moiety and an acidic moiety, zwitterions (“inner salts”) may be formed and are included within the term “salt(s)” as used herein. Pharmaceutically acceptable (i.e., non-toxic, physiologically acceptable) salts are used, although other salts are also useful, e.g., in isolation or purification steps which may be employed during preparation. Salts of the compounds of Formula I, II or III may be formed, for example, by reacting a compound with an amount of acid or base, such as an equivalent amount, in a medium such as one in which the salt precipitates or in an aqueous medium followed by lyophilization.

The ligands which contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

The ligands which contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine and the like.

Non-limiting examples of the ligands for the inducible gene expression system utilizing the FK506 binding domain are FK506, Cyclosporin A, or Rapamycin. FK506, rapamycin, and their analogs are disclosed in U.S. Pat. Nos. 6,649,595 B2 and 6,187,757. See also U.S. Pat. Nos. 7,276,498 and 7,273,874.

A LDTF such as an EcR complex can be activated by an active ecdysteroid or non-steroidal ligand bound to one of the proteins of the complex, inclusive of EcR, but not excluding other proteins of the complex. A LDTF such as an EcR complex includes proteins which are members of the nuclear receptor superfamily wherein all members are characterized by the presence of one or more polypeptide subunits comprising an amino-terminal transactivation domain (“AD,” “TD,” or “TA,” used interchangeably herein), a DNA binding domain (“DBD”), and a ligand binding domain (“LBD”). The AD may be present as a fusion with a “heterodimerization partner” or “HP.” A fusion protein comprising an AD and HP of the invention is referred to herein as a “coactivation protein” or “CAP.” The DBD and LBD may be expressed as a fusion protein, referred to herein as a “ligand-inducible transcription factor (“LTF”). The fusion partners may be separated by a linker, e.g., a hinge region. Some members of the LTF family may also have another transactivation domain on the carboxy-terminal side of the LBD. The DBD is characterized by the presence of two cysteine zinc fingers between which are two amino acid motifs, the P-box and the D-box, which confer specificity for ecdysone response elements. These domains may be either native, modified, or chimeras of different domains of heterologous receptor proteins.

The DNA sequences making up the exogenous gene, the response element, and the LDTF, e.g., EcR complex, may be incorporated into archaebacteria, prokaryotic cells such as Escherichia coli, Bacillus subtilis, or other enterobacteria, or eukaryotic cells such as plant or animal cells. However, because many of the proteins expressed by the gene are processed incorrectly in bacteria, eucaryotic cells are preferred. The cells may be in the form of single cells or multicellular organisms. The nucleotide sequences for the exogenous gene, the response element, and the receptor complex can also be incorporated as RNA molecules, preferably in the form of functional viral RNAs such as tobacco mosaic virus. Of the eukaryotic cells, vertebrate cells are preferred because they naturally lack the molecules which confer responses to the ligands of this invention for the EcR. As a result, they are “substantially insensitive” to the ligands of this invention. Thus, the ligands useful in this invention will have negligible physiological or other effects on transformed cells, or the whole organism. Therefore, cells can grow and express the desired product, substantially unaffected by the presence of the ligand itself.

The term “ecdysone receptor complex” generally refers to a heterodimeric protein complex having at least two members of the nuclear receptor family, ecdysone receptor (“EcR”) and ultraspiracle (“USP”) proteins (see Yao et al., Nature 366:476 (1993)); Yao et al., Cell 71:63 (1992)). The functional EcR complex may also include additional protein(s) such as immunophilins. Additional members of the nuclear receptor family of proteins, known as transcriptional factors (such as DHR38, betaFTZ-1 or other insect homologs), may also be ligand dependent or independent partners for EcR and/or USP. The EcR complex can also be a heterodimer of EcR protein and the vertebrate homolog of ultraspiracle protein, retinoic acid-X-receptor (“RXR”) protein or a chimera of USP and RXR. The term EcR complex also encompasses homodimer complexes of the EcR protein or USP.

An EcR complex can be activated by an active ecdysteroid or non-steroidal ligand bound to one of the proteins of the complex, inclusive of EcR, but not excluding other proteins of the complex. As used herein, the term “ligand,” as applied to EcR-based gene switches, describes small and soluble molecules having the capability of activating a gene switch to stimulate expression of a polypeptide encoded therein. Examples of ligands include, without limitation, an ecdysteroid, such as ecdysone, 20-hydroxyecdysone, ponasterone A, muristerone A, and the like, 9-cis-retinoic acid, synthetic analogs of retinoic acid, N,N′-diacylhydrazines such as those disclosed in U.S. Pat. Nos. 6,013,836; 5,117,057; 5,530,028; and 5,378,726 and U.S. Published Application Nos. 2005/0209283 and 2006/0020146; oxadiazolines as described in U.S. Published Application No. 2004/0171651; dibenzoylalkyl cyanohydrazines such as those disclosed in European Application No. 461,809; N-alkyl-N,N′-diaroylhydrazines such as those disclosed in U.S. Pat. No. 5,225,443; N-acyl-N-alkylcarbonylhydrazines such as those disclosed in European Application No. 234,994; N-aroyl-N-alkyl-N′-aroylhydrazines such as those described in U.S. Pat. No. 4,985,461; amidoketones such as those described in U.S. Published Application No. 2004/0049037; and other similar materials including 3,5-di-tert-butyl-4-hydroxy-N-isobutyl-benzamide, 8-O-acetylharpagide, oxysterols, 22(R) hydroxycholesterol, 24(S) hydroxycholesterol, 25-epoxycholesterol, T0901317, 5-alpha-6-alpha-epoxycholesterol-3-sulfate (ECHS), 7-ketocholesterol-3-sulfate, famesol, bile acids, 1,1-biphosphonate esters, juvenile hormone III, and the like. Examples of diacylhydrazine ligands useful in the invention include RG-115819 (3,5-Dimethylbenzoic acid N-(1-ethyl-2,2-dimethyl-propyl)-N′-(2-methyl-3-methoxy-benzoyl)hydrazide), RG-115932 ((R)-3,5-Dimethyl-benzoic acid N-(1-tert-butyl-butyl)-N′-(2-ethyl-3-methoxy-benzoyl)-hydrazide), and RG-115830 (3,5-Dimethyl-benzoic acid N-(1-tert-butyl-butyl)-N′-(2-ethyl-3-methoxy-benzoyl)-hydrazide). See U.S. application Ser. No. 12/155,111, filed May 29, 2008, and PCT/US2008/006757 filed May 29, 2008, for additional diacylhydrazines that are useful in the practice of the invention.

The EcR complex includes proteins which are members of the nuclear receptor superfamily wherein all members are characterized by the presence of an amino-terminal transactivation domain (“TA”), a DNA binding domain (“DBD”), and a ligand binding domain (“LBD”) separated by a hinge region. Some members of the family may also have another transactivation domain on the carboxy-terminal side of the LBD. The DBD is characterized by the presence of two cysteine zinc fingers between which are two amino acid motifs, the P-box and the D-box, which confer specificity for ecdysone response elements. These domains may be either native, modified, or chimeras of different domains of heterologous receptor proteins.

The DNA sequences making up the exogenous gene, the response element, and the EcR complex may be incorporated into archaebacteria, procaryotic cells such as Escherichia coli, Bacillus subtilis, or other enterobacteria, or eucaryotic cells such as plant or animal cells. However, because many of the proteins expressed by the gene are processed incorrectly in bacteria, eucaryotic cells are preferred. The cells may be in the form of single cells or multicellular organisms. The nucleotide sequences for the exogenous gene, the response element, and the receptor complex can also be incorporated as RNA molecules, preferably in the form of functional viral RNAs such as tobacco mosaic virus. Of the eucaryotic cells, vertebrate cells are preferred because they naturally lack the molecules which confer responses to the ligands of this invention for the EcR. As a result, they are “substantially insensitive” to the ligands of this invention. Thus, the ligands useful in this invention will have negligible physiological or other effects on transformed cells, or the whole organism. Therefore, cells can grow and express the desired product, substantially unaffected by the presence of the ligand itself.

EcR ligands, when used with the EcR complex which in turn is bound to the response element linked to an exogenous gene (e.g., IL-12), provide the means for external temporal regulation of expression of the exogenous gene. The order in which the various components bind to each other, that is, ligand to receptor complex and receptor complex to response element, is not critical. Typically, modulation of expression of the exogenous gene is in response to the binding of the EcR complex to a specific control, or regulatory, DNA element. The EcR protein, like other members of the nuclear receptor family, possesses at least three domains, a transactivation domain, a DNA binding domain, and a ligand binding domain. This receptor, like a subset of the nuclear receptor family, also possesses less well-defined regions responsible for heterodimerization properties. Binding of the ligand to the ligand binding domain of EcR protein, after heterodimerization with USP or RXR protein, enables the DNA binding domains of the heterodimeric proteins to bind to the response element in an activated form, thus resulting in expression or suppression of the exogenous gene. This mechanism does not exclude the potential for ligand binding to either EcR or USP, and the resulting formation of active homodimer complexes (e.g., EcR+EcR or USP+USP). In one embodiment, one or more of the receptor domains can be varied producing a chimeric gene switch. Typically, one or more of the three domains may be chosen from a source different than the source of the other domains so that the chimeric receptor is optimized in the chosen host cell or organism for transactivating activity, complementary binding of the ligand, and recognition of a specific response element. In addition, the response element itself can be modified or substituted with response elements for other DNA binding protein domains such as the GAL-4 protein from yeast (see Sadowski et al., Nature 335:563 (1988) or LexA protein from E. coli (see Brent et al., Cell 43:729 (1985)) to accommodate chimeric EcR complexes. Another advantage of chimeric systems is that they allow choice of a promoter used to drive the exogenous gene according to a desired end result. Such double control can be particularly important in areas of gene therapy, especially when cytotoxic proteins are produced, because both the timing of expression as well as the cells wherein expression occurs can be controlled. When exogenous genes, operatively linked to a suitable promoter, are introduced into the cells of the subject, expression of the exogenous genes is controlled by the presence of the ligand of this invention. Promoters may be constitutively or inducibly regulated or may be tissue-specific (that is, expressed only in a particular cell type) or specific to certain developmental stages of the organism.

In certain embodiments, the therapeutic switch promoter described in the methods is constitutive. In certain embodiments, the therapeutic switch promoter is activated under conditions associated with a disease, disorder, or condition, e.g., the promoter is activated in response to a disease, in response to a particular physiological, developmental, differentiation, or pathological condition, and/or in response to one or more specific biological molecules; and/or the promoter is activated in particular tissue or cell types. In certain embodiments, the disease, disorder, or condition is responsive to the therapeutic polypeptide or polynucleotide. For example, in certain non-limiting embodiments the therapeutic polynucleotide or polypeptide is useful to treat, prevent, ameliorate, reduce symptoms, prevent progression, or cure the disease, disorder or condition, but need not accomplish any one or all of these things. In certain embodiments, the first and second polynucleotides are introduced to permit expression of the ligand-dependent transcription factor complex under conditions associated with a disease, disorder or condition. In one embodiment, the therapeutic methods are carried out such that the therapeutic polypeptide or therapeutic polynucleotide is expressed and disseminated through the subject at a level sufficient to treat, ameliorate, or prevent said disease, disorder, or condition. As used herein, “disseminated” means that the polypeptide is expressed and released from the modified cell sufficiently to have an effect or activity in the subject. Dissemination may be systemic, local or anything in between. For example, the therapeutic polypeptide or therapeutic polynucleotide might be systemically disseminated through the bloodstream or lymph system. Alternatively, the therapeutic polypeptide or therapeutic polynucleotide might be disseminated locally in a tissue or organ to be treated.

Numerous genomic and cDNA nucleic acid sequences coding for a variety of polypeptides, such as transcription factors and reporter proteins, are well known in the art. Those skilled in the art have access to nucleic acid sequence information for virtually all known genes and can either obtain the nucleic acid molecule directly from a public depository, the institution that published the sequence, or employ routine methods to prepare the molecule. See for example the description of the sequence accession numbers, infra.

The gene switch may be any gene switch system that regulates gene expression by addition or removal of a specific ligand. In one embodiment, the gene switch is one in which the level of gene expression is dependent on the level of ligand that is present. Examples of ligand-dependent transcription factors that may be used in the gene switches of the invention include, without limitation, members of the nuclear receptor superfamily activated by their respective ligands (e.g., glucocorticoid, estrogen, progestin, retinoid, ecdysone, and analogs and mimetics thereof) and rTTA activated by tetracycline. In one aspect of the invention, the gene switch is an EcR-based gene switch. Examples of such systems include, without limitation, the systems described in U.S. Pat. Nos. 6,258,603, 7,045,315, U.S. Published Patent Application Nos. 2006/0014711, 2007/0161086, and International Published Application No. WO 01/70816. Examples of chimeric ecdysone receptor systems are described in U.S. Pat. No. 7,091,038, U.S. Published Patent Application Nos. 2002/0110861, 2004/0033600, 2004/0096942, 2005/0266457, and 2006/0100416, and International Published Application Nos. WO 01/70816, WO 02/066612, WO 02/066613, WO 02/066614, WO 02/066615, WO 02/29075, and WO 2005/108617. An example of a non-steroidal ecdysone agonist-regulated system is the RheoSwitch® Mammalian Inducible Expression System (New England Biolabs, Ipswich, Mass.).

In one embodiment, a polynucleotide encoding the gene switch comprises a single transcription factor sequence encoding a ligand-dependent transcription factor under the control of a promoter. The transcription factor sequence may encode a ligand-dependent transcription factor that is a naturally occurring or an artificial transcription factor. An artificial transcription factor is one in which the natural sequence of the transcription factor has been altered, e.g., by mutation of the sequence or by the combining of domains from different transcription factors. In one embodiment, the transcription factor comprises a Group H nuclear receptor ligand binding domain (LBD). In one embodiment, the Group H nuclear receptor LBD is from an EcR, a ubiquitous receptor, an orphan receptor 1, a NER-1, a steroid hormone nuclear receptor 1, a retinoid X receptor interacting protein-15, a liver X receptor β, a steroid hormone receptor like protein, a liver X receptor, a liver X receptor α, a farnesoid X receptor, a receptor interacting protein 14, or a farnesol receptor. In another embodiment, the Group H nuclear receptor LBD is from an ecdysone receptor.

The EcR and the other Group H nuclear receptors are members of the nuclear receptor superfamily wherein all members are generally characterized by the presence of an amino-terminal transactivation domain (TD), a DNA binding domain (DBD), and a LBD separated from the DBD by a hinge region. As used herein, the term “DNA binding domain” comprises a minimal polypeptide sequence of a DNA binding protein, up to the entire length of a DNA binding protein, so long as the DNA binding domain functions to associate with a particular response element. Members of the nuclear receptor superfamily are also characterized by the presence of four or five domains: A/B, C, D, E, and in some members F (see U.S. Pat. No. 4,981,784 and Evans, Science 240:889 (1988)). The “A/B” domain corresponds to the transactivation domain, “C” corresponds to the DNA binding domain, “D” corresponds to the hinge region, and “E” corresponds to the ligand binding domain. Some members of the family may also have another transactivation domain on the carboxy-terminal side of the LBD corresponding to “F”.

The DBD is characterized by the presence of two cysteine zinc fingers between which are two amino acid motifs, the P-box and the D-box, which confer specificity for response elements. These domains may be either native, modified, or chimeras of different domains of heterologous receptor proteins. The EcR, like a subset of the nuclear receptor family, also possesses less well-defined regions responsible for heterodimerization properties. Because the domains of nuclear receptors are modular in nature, the LBD, DBD, and TD may be interchanged.

In another embodiment, the transcription factor comprises a TD, a DBD that recognizes a response element associated with the exogenous gene whose expression is to be modulated; and a Group H nuclear receptor LBD. In certain embodiments, the Group H nuclear receptor LBD comprises a substitution mutation.

In another embodiment, a polynucleotide encoding the gene switch comprises a first transcription factor sequence under the control of a first promoter and a second transcription factor sequence under the control of a second promoter, wherein the proteins encoded by said first transcription factor sequence and said second transcription factor sequence interact to form a protein complex which functions as a ligand-dependent transcription factor, i.e., a “dual switch”- or “two-hybrid”-based gene switch. The first and second promoters may be the same or different.

In certain embodiments, the polynucleotide encoding a gene switch comprises a first transcription factor sequence and a second transcription factor sequence under the control of a promoter, wherein the proteins encoded by said first transcription factor sequence and said second transcription factor sequence interact to form a protein complex which functions as a ligand-dependent transcription factor, i.e., a “single gene switch”. The first transcription factor sequence and a second transcription factor sequence may be connected by an internal ribosomal entry site (IRES). The IRES may be an EMCV TRES.

In one embodiment, the first transcription factor sequence encodes a polypeptide comprising a TD, a DBD that recognizes a response element associated with the exogenous gene whose expression is to be modulated; and a Group H nuclear receptor LBD, and the second transcription factor sequence encodes a transcription factor comprising a nuclear receptor LBD selected from a vertebrate RXR LBD, an invertebrate RXR LBD, an ultraspiracle protein LBD, and a chimeric LBD comprising two polypeptide fragments, wherein the first polypeptide fragment is from a vertebrate RXR LBD, an invertebrate RXR LBD, or an ultraspiracle protein LBD, and the second polypeptide fragment is from a different vertebrate RXR LBD, invertebrate RXR LBD, or ultraspiracle protein LBD.

In another embodiment, the gene switch comprises a first transcription factor sequence encoding a first polypeptide comprising a nuclear receptor LBD and a DBD that recognizes a response element associated with the exogenous gene whose expression is to be modulated, and a second transcription factor sequence encoding a second polypeptide comprising a TD and a nuclear receptor LBD, wherein one of the nuclear receptor LBDs is a Group H nuclear receptor LBD. In one embodiment, the first polypeptide is substantially free of a TD and the second polypeptide is substantially free of a DBD. For purposes of the invention, “substantially free” means that the protein in question does not contain a sufficient sequence of the domain in question to provide activation or binding activity.

In another aspect of the invention, the first transcription factor sequence encodes a protein comprising a heterodimer partner and a TD and the second transcription factor sequence encodes a protein comprising a DBD and a LBD.

When only one nuclear receptor LBD is a Group H LBD, the other nuclear receptor LBD may be from any other nuclear receptor that forms a dimer with the Group H LBD. For example, when the Group H nuclear receptor LBD is an EcR LBD, the other nuclear receptor LBD “partner” may be from an EcR, a vertebrate RXR, an invertebrate RXR, an ultraspiracle protein (USP), or a chimeric nuclear receptor comprising at least two different nuclear receptor LBD polypeptide fragments selected from a vertebrate RXR, an invertebrate RXR, and a USP (see WO 01/70816 A2, International Patent Application No. PCT/US02/05235 and US 2004/0096942 A1). The “partner” nuclear receptor ligand binding domain may further comprise a truncation mutation, a deletion mutation, a substitution mutation, or another modification.

In one embodiment, the vertebrate RXR LBD is from a human Homo sapiens, mouse Mus musculus, rat Rattus norvegicus, chicken Gallus gallus, pig Sus scrofa domestica, frog Xenopus laevis, zebrafish Danio rerio, tunicate Polyandrocarpa misakiensis, or jellyfish Tripedalia cysophora RXR.

In one embodiment, the invertebrate RXR ligand binding domain is from a locust Locusta migratoria ultraspiracle polypeptide (“LmUSP”), an ixodid tick Amblyomma americanum RXR homolog 1 (“AmaRXR1”), an ixodid tick Amblyomma americanum RXR homolog 2 (“AmaRXR2”), a fiddler crab Celuca pugilator RXR homolog (“CpRXR”), a beetle Tenebrio molitor RXR homolog (“TmRXR”), a honeybee Apis mellifera RXR homolog (“AmRXR”), an aphid Myzus persicae RXR homolog (“MpRXR”), or a non-Dipteran/non-Lepidopteran RXR homolog.

In one embodiment, the chimeric RXR LBD comprises at least two polypeptide fragments selected from a vertebrate species RXR polypeptide fragment, an invertebrate species RXR polypeptide fragment, and a non-Dipteran/non-Lepidopteran invertebrate species RXR homolog polypeptide fragment. A chimeric RXR ligand binding domain for use in the invention may comprise at least two different species RXR polypeptide fragments, or when the species is the same, the two or more polypeptide fragments may be from two or more different isoforms of the species RXR polypeptide fragment.

In one embodiment, the chimeric RXR ligand binding domain comprises at least one vertebrate species RXR polypeptide fragment and one invertebrate species RXR polypeptide fragment.

In another embodiment, the chimeric RXR ligand binding domain comprises at least one vertebrate species RXR polypeptide fragment and one non-Dipteran/non-Lepidopteran invertebrate species RXR homolog polypeptide fragment.

The ligand, when combined with the LBD of the nuclear receptor(s), which in turn are bound to the response element linked to the exogenous gene, provides external temporal regulation of expression of the exogenous gene. The binding mechanism or the order in which the various components of this invention bind to each other, that is, for example, ligand to LBD, DBD to response element, TD to promoter, etc., is not critical.

In a specific example, binding of the ligand to the LBD of a Group H nuclear receptor and its nuclear receptor LBD partner enables expression of the exogenous gene. This mechanism does not exclude the potential for ligand binding to the Group H nuclear receptor (GHNR) or its partner, and the resulting formation of active homodimer complexes (e.g., GHNR+GHNR or partner+partner). Preferably, one or more of the receptor domains is varied producing a hybrid gene switch. Typically, one or more of the three domains, DBD, LBD, and TD, may be chosen from a source different than the source of the other domains so that the hybrid genes and the resulting hybrid proteins are optimized in the chosen host cell or organism for transactivating activity, complementary binding of the ligand, and recognition of a specific response element. In addition, the response element itself can be modified or substituted with response elements for other DNA binding protein domains such as the GAL-4 protein from yeast (see Sadowski et al., Nature 335:563 1988) or LexA protein from Escherichia coli (see Brent et al., Cell 43:729 1985), or synthetic response elements specific for targeted interactions with proteins designed, modified, and selected for such specific interactions (see, for example, Kim et al., Proc. Natl. Acad. Sci. USA, 94:3616 1997) to accommodate hybrid receptors.

The functional EcR complex may also include additional protein(s) such as immunophilins. Additional members of the nuclear receptor family of proteins, known as transcriptional factors (such as DHR38 or betaFTZ-1), may also be ligand dependent or independent partners for EcR, USP, and/or RXR. Additionally, other cofactors may be required such as proteins generally known as coactivators (also termed adapters or mediators). These proteins do not bind sequence-specifically to DNA and are not involved in basal transcription. They may exert their effect on transcription activation through various mechanisms, including stimulation of DNA-binding of activators, by affecting chromatin structure, or by mediating activator-initiation complex interactions. Examples of such coactivators include RIP140, TIF1, RAP46/Bag-1, ARA70, SRC-1/NCoA-1, TIF2/GRIP/NCoA-2, ACTR/AIB1/RAC3/pCIP as well as the promiscuous coactivator C response element B binding protein, CBP/p300 (for review see Glass et al., Curr. Opin. Cell Biol. 9:222 1997). Also, protein cofactors generally known as corepressors (also known as repressors, silencers, or silencing mediators) may be required to effectively inhibit transcriptional activation in the absence of ligand. These corepressors may interact with the unliganded EcR to silence the activity at the response element. Current evidence suggests that the binding of ligand changes the conformation of the receptor, which results in release of the corepressor and recruitment of the above described coactivators, thereby abolishing their silencing activity. Examples of corepressors include N—CoR and SMRT (for review, see Horwitz et al., Mol Endocrinol. 10:1167 1996). These cofactors may either be endogenous within the cell or organism, or may be added exogenously as transgenes to be expressed in either a regulated or unregulated fashion.

Vectors with Inducible Expression of Interleukin 12

As used herein, the term “rAD.RheoIL12” or “Ad-RTS-mIL-12” or “Ad-RTS-hIL-12” refers to an adenoviral polynucleotide vector harboring the human IL-12 (hIL-12) gene or a mouse IL-12 (mIL-12) gene under the control of a gene switch of the RheoSwitch© Therapeutic System (RTS©), which can produce IL-12 protein in the presence of activating ligand. As used herein, the term “rAd.cIL12” refers to an adenoviral polynucleotide control vector containing the IL-12 gene under the control of a constitutive promoter.

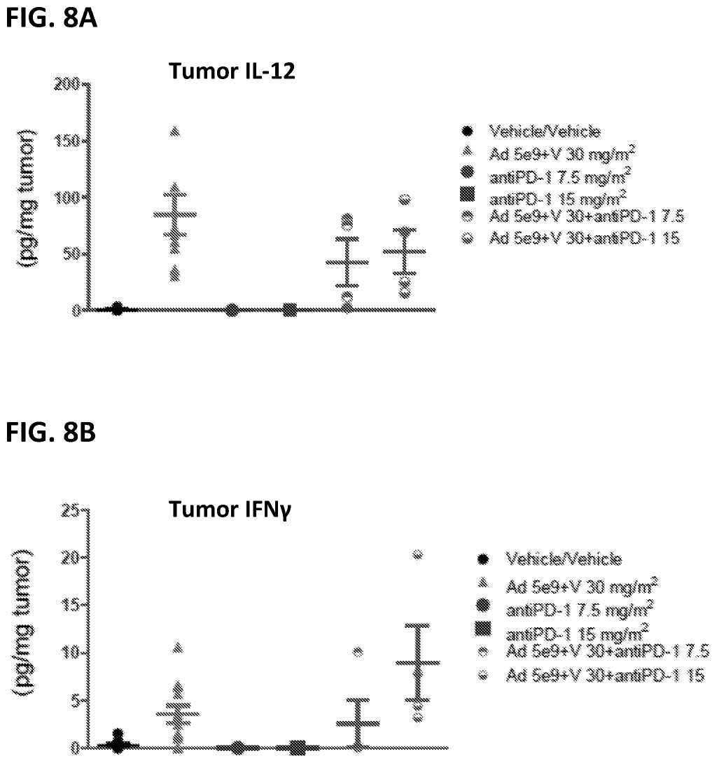

The recombinant DNA used as the recombinant adenoviral vector allows the expression of human IL-12 and one or more other immunodulators under the control of the RheoSwitch® Therapeutic System (RTS®). The RTS® comprises a bicistronic message expressed from the human Ubiquitin C promoter and codes for two fusion proteins: Gal4-EcR and VP16-RXR. Gal4-EcR is a fusion between the DNA binding domain (amino acids 1-147) of yeast Gal4 and the DEF domains of the ecdysone receptor from the insect Choristoneura fumiferana. In another embodiment, the RTS® consists of a bicistronic message expressed from the human Ubiquitin C promoter and codes for two fusion proteins: Gal4-EcR and VP16-RXR. Gal4-EcR is a fusion between the DNA binding domain (amino acids 1-147) of yeast Gal4 and the DEF domains of the ecdysone receptor from the insect Choristoneura fumiferana. VP16-RXR is a fusion between the transcription activation domain of HSV-VP16 and the EF domains of a chimeric RXR derived from human and locust sequences. These Gal4-EcR and VP16-RXR sequences are separated by an internal ribosome entry site (IRES) from EMCV. These two fusion proteins dimerize when Gal4-EcR binds to a small molecule drug (RG-115932) and activate transcription of hIL-12 and one or more other immunodulators from a Gal4-responsive promoter that contains six Gal4-binding sites and a synthetic minimal promoter. The RTS transcription unit described above is placed downstream of the hIL-12 and one or more other immunodulators transcription units. This whole RTS-hIL12-immunomodulator cassette is incorporated into the adenovirus 5 genome at the site where the E1 region has been deleted. The adenoviral backbone also lacks the E3 gene. A map for the adenoviral vector Ad-RTS-hIL-12 is shown in FIG. 8 of US 2009/0123441 A1.

In some embodiments, the IL-12 p40 of the disclosure comprise the amino acid sequence of:

(SEQ ID NO: 12)
MCPQKLTISWFAIVLLVSPLMAMWELEKDVYVVEVDWTPDAPGETVNLT
CDTPEEDDITWTSDQRHGVIGSGKTLTITVKEFLDAGQYTCHKGGETLS
HSHLLLHKKENGIWSTEILKNFKNKTFLKCEAPNYSGRFTCSWLVQRNM
DLKFNIKSSSSSPDSRAVTCGMASLSAEKVTLDQRDYEKYSVSCQEDVT
CPTAEETLPIELALEARQQNKYENYSTSFFIRDIIKPDPPKNLQMKPLK
NSQVEVSWEYPDSWSTPHSYFSLKFFVRIQRKKEKMKETEEGCNQKGAF
LVEKTSTEVQCKGGNVCVQAQDRYYNSSCSKWACVPCRVRS.

In some embodiments, the IL-12 p40 of the disclosure is encoded by the polynucleotide sequence of:

| (SEQ ID NO: 13) | |
|---|---|
| atgtgccccc agaagctgac catcagctgg ttcgccatcg tgctgctggt gagccccctg | 60 |
| atggccatgt gggagctgga gaaggacgtg tacgtggtgg aggtggactg gacccccgac | 120 |
| gcccccggcg agaccgtgaa cctgacttgc gacacccccg aggaggacga catcacctgg | 180 |
| accagcgacc agagacacgg cgtcatcggc agcggcaaga ccctgaccat caccgtgaag | 240 |
| gagttcctgg acgccggaca gtacacctgt cacaagggcg gcgagaccct gagccacagc | 300 |
| cacctgttgc tgcacaagaa ggagaacggc atctggagca ccgagatcct gaagaacttc | 360 |
| aagaacaaga ccttcctgaa gtgcgaggcc cccaactaca gcggcagatt cacctgtagc | 420 |
| tggctggtgc agagaaacat ggacctgaag ttcaacatca agagcagcag cagcagcccc | 480 |
| gacagcagag ccgtgacatg cggcatggcc agcctgagcg ccgagaaggt gaccctggac | 540 |
| cagagagact acgagaagta cagcgtgagc tgccaggagg acgtgacctg tcccaccgcc | 600 |
| gaggagaccc tgcccatcga gcttgccctg gaagccagac agcagaacaa gtacgagaac | 660 |
| tacagcacca gcttcttcat cagagacatc atcaagcccg acccccccaa gaacctccag | 720 |
| atgaagcccc tgaagaacag ccaggtggag gtgtcctggg agtaccccga cagctggagc | 780 |
| accccccaca gctacttcag cctgaagttc ttcgtgagaa tccagagaaa gaaggagaag | 840 |
| atgaaggaga ccgaggaggg ctgcaaccag aagggcgctt tcctggtgga gaaaaccagc | 900 |
| accgaggtgc agtgcaaggg cggcaacgtg tgtgtgcagg cccaggacag atactacaac | 960 |
| agcagctgct ccaagtgggc ctgcgtgccc tgccgcgtga gaagctga. | 1008 |

In some embodiments, the IL-12 p40 of the disclosure has an amino acid sequence having at least 75%, 80%, 85%, 90%, 95%, 97%, 99%, or any percentage in between of identity to the amino acid sequence of:

(SEQ ID NO: 12)
MCPQKLTISWFAIVLLVSPLMAMWELEKDVYVVEVDWTPDAPGETVNLT
CDTPEEDDITWTSDQRHGVIGSGKTLTITVKEFLDAGQYTCHKGGETLS
HSHLLLHKKENGIWSTEILKNFKNKTFLKCEAPNYSGRFTCSWLVQRNM
DLKFNIKSSSSSPDSRAVTCGMASLSAEKVTLDQRDYEKYSVSCQEDVT
CPTAEETLPIELALEARQQNKYENYSTSFFIRDIIKPDPPKNLQMKPLK
NSQVEVSWEYPDSWSTPHSYFSLKFFVRIQRKKEKMKETEEGCNQKGAF
LVEKTSTEVQCKGGNVCVQAQDRYYNSSCSKWACVPCRVRS.

In some embodiments, the IL-12 p35 of the disclosure comprise the amino acid sequence of:

(SEQ ID NO: 14)
MCQSRYLLFLATLALLNHLSLARVIPVSGPARCLSQSRNLLKTTDDMVK
TAREKLKHYSCTAEDIDHEDITRDQTSTLKTCLPLELHKNESCLATRET
SSTTRGSCLPPQKTSLMMTLCLGSIYEDLKMYQTEFQAINAALQNHNHQ
QIILDKGMLVAIDELMQSLNHNGETLRQKPPVGEADPYRVKMKLCILLH
AFSTRVVTINRVMGYLSSA

In some embodiments, the IL-12 p35 of the disclosure is encoded by the polynucleotide sequence of:

| (SEQ ID NO: 15) | |
|---|---|
| atgtgccaga gcagatacct gttgttcctg gctaccctgg ccctgctgaa ccacctgagc | 60 |
| ctggcccgcg tgatccccgt gagcggcccc gccagatgcc tgagccagag cagaaacctg | 120 |
| ttgaaaacaa ccgacgacat ggtgaaaacc gccagagaga agctgaagca ctacagctgc | 180 |
| accgccgagg acatcgacca cgaggacatc accagagacc agaccagcac cctgaaaacc | 240 |
| tgtctgcccc tggagctgca caagaacgag agctgcctgg ctaccagaga gaccagcagc | 300 |
| accaccagag gcagctgcct gcccccccag aaaaccagcc tgatgatgac cctgtgcctg | 360 |
| ggcagcatct acgaggacct gaagatgtac cagaccgagt tccaggccat caacgccgcc | 420 |
| ctgcaaaacc acaaccacca gcagatcatc ctggacaagg gcatgttggt ggccatcgac | 480 |
| gagctgatgc agagcctgaa ccacaacggc gagaccctga gacagaagcc ccccgtgggc | 540 |
| gaggccgacc cctacagagt gaagatgaag ctgtgcatcc tgctgcacgc cttcagcacc | 600 |
| agagtggtga ccatcaacag agtgatgggc tacctgagca gcgcctga. | 648 |

In some embodiments, the IL-12 p35 of the disclosure has an amino acid sequence having at least 75%, 80%, 85%, 90%, 95%, 97%, 99%, or any percentage in between of identity to the amino acid sequence of:

(SEQ ID NO: 14)
MCQSRYLLFLATLALLNHLSLARVIPVSGPARCLSQSRNLLKTTDDMVK
TAREKLKHYSCTAEDIDHEDITRDQTSTLKTCLPLELHKNESCLATRET
SSTTRGSCLPPQKTSLMMTLCLGSIYEDLKMYQTEFQAINAALQNHNHQ
QIILDKGMLVAIDELMQSLNHNGETLRQKPPVGEADPYRVKMKLCILLH
AFSTRVVTINRVMGYLSSA

As used herein, the term “IL-12p70” refers to IL-12 protein, which naturally has two subunits commonly referred to as p40 and p35. The term IL-12p70 encompasses fusion proteins comprising the two subunits of IL-12 (IL-12 p40 and IL-12 p35), wherein the fusion protein may include linker amino acids between subunits.

In one embodiment, the recombinant adenoviral vector contains the following exemplary regulatory elements in addition to the viral vector sequences: Human Ubiquitin C promoter, Internal ribosome entry site derived from EMCV, an inducible promoter containing 6 copies of Gal4-binding site, 3 copies of SP-1 binding sites, and a synthetic minimal promoter sequence, SV40 polyadenylation sites, and a transcription termination sequence derived from human alpha-globin gene. It should be understood that other regulatory elements could be utilized as alternatives.

In one embodiment, the recombinant adenoviral vector Ad-RTS-hIL-12-immunomodulator(s) is produced in the following manner. The coding sequences for the receptor fusion proteins, VP16-RXR and Gal4-EcR separated by the EMCV-IRES (internal ribosome entry site), are inserted into the adenoviral shuttle vector under the control of the human ubiquitin C promoter (constitutive promoter). Subsequently, the coding sequences for the p40 and p35 subunits of hIL-12 separated by IRES, and one or more other immunomodulators, is placed under the control of a synthetic inducible promoter containing 6 copies of Gal4-binding site are inserted upstream of the ubiquitin C promoter and the receptor sequences. The shuttle vector contains the adenovirus serotype 5 sequences from the left end to map unit 16 (mu16), from which the E1 sequences are deleted and replaced by the RTS, IL-12 and one or more other immunomodulator sequences (RTS-hIL-12). The shuttle vector carrying the RTS-hIL12-immunodulator(s) is tested by transient transfection in HT-1080 cells for Activator Drug-dependent IL-12 and other immunomodulator(s) expression. The shuttle vector is then recombined with the adenoviral backbone by cotransfection into HEK 293 cells to obtain recombinant adenovirus Ad-RTS-hIL-12-immunomodulator(s). The adenoviral backbone contains sequence deletions of mu 0 to 9.2 at the left end of the genome and the E3 gene. The shuttle vector and the adenoviral backbone contain the overlapping sequence from mu 9.2 to mu 16 that allows the recombination between them and production of the recombinant adenoviral vector. Since the recombinant adenoviral vector is deficient in the E1 and E3 regions, the virus is replication-deficient in normal mammalian cells. However, the virus can replicate in HEK 293 cells that harbor the adenovirus-5 E1 region and hence provide the E1 function in trans.

In certain embodiments, Ad-RTS-hIL12 and components thereof are encoded by polynucleotide and polypeptide sequences as described and disclosed in:

SEQ ID NOs: 1-64 in WO2001/070816 (PCT/US2001/09050) filed 21 Mar. 2001;

SEQ ID NOs: 1-113 in WO2002/066612 (PCT/US2002/005090) filed 20 Feb. 2002;

SEQ ID NOs: 1-75 in WO2002/066614 (PCT/US2002/005706) filed 20 Feb. 2002;

SEQ ID NOs: 1-8 and 13 in WO2009/048560 (PCT/US2008/011563) filed 8 Oct. 2008;

SEQ ID NOs: 1-24 and 29 in WO2010/042189 (PCT/US2009/005510) filed 8 Oct. 2009; and,

SEQ ID NOs: 1-6, 24-29, 47-62 in WO2011/119773 (PCT/US2011/029682) filed 23 Mar. 2011.

The disclosure and sequences from the sequence listings in each of the above referenced publications are hereby incorporated by reference in the entirety.

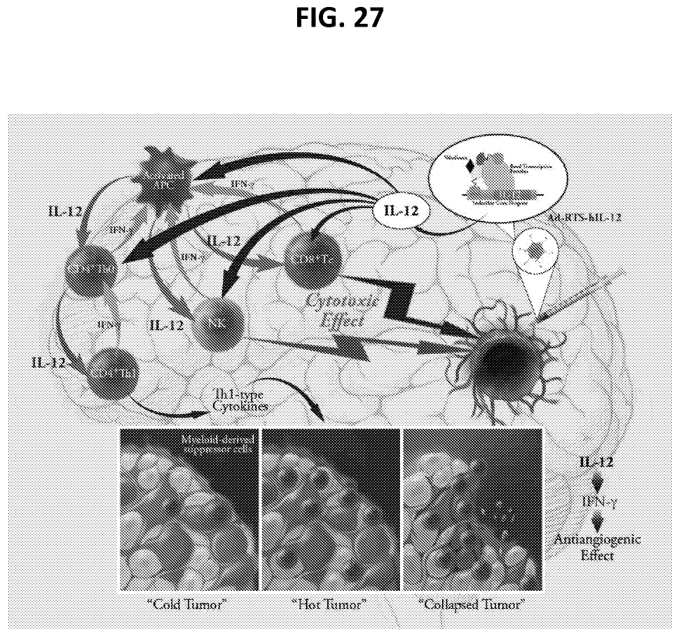

The bioactivities of IL-12 are also well known and include, without limitation, differentiation of naive T cells into Th1 cells, stimulation of the growth and function of T cells, production of interferon-gamma (IFN-gamma) and tumor necrosis factor-alpha (TNF-α) from T and natural killer (NK) cells, reduction of IL-4 mediated suppression of IFN-gamma, enhancement of the cytotoxic activity of NK cells and CD8⁺ cytotoxic T lymphocytes, stimulation of the expression of IL-12R-31 and IL-12R-132, facilitation of the presentation of tumor antigens through the upregulation of MHC I and II molecules, and anti-angiogenic activity. FIG. 27 is a schematic illustration of the aforementioned cytokine cascade. The term “a protein having the function of IL-12” encompasses mutants of a wild type IL-12 sequence, wherein the wild type sequence has been altered by one or more of addition, deletion, or substitution of amino acids, as well as non-IL-12 proteins that mimic one or more of the bioactivities of IL-12.

In one embodiment, a nucleic acid adenoviral vector is provided containing a gene switch, wherein the coding sequences for VP16-RXR and Gal4-EcR are separated by the EMCV internal ribosome entry site (IRES) sequence are inserted into the adenoviral shuttle vector under the control of the human ubiquitin C promoter. For example, the coding sequences for the p40 and p35 subunits of IL-12 separated by an IRES sequence and placed under the control of a synthetic inducible promoter, are inserted upstream of the ubiquitin C promoter. In another example, the coding sequence of TNF-alpha, which is placed under the control of a synthetic inducible promoter, is inserted upstream of the ubiquitin C promoter.

In another embodiment, the invention provides a shuttle vector carrying transcription units (VP16-RXR and Gal4-EcR) for the two fusion proteins and inducible IL-12 subunits recombined with the adenoviral backbone (AdEasyl) in E. coli BJ5183 cells. After verifying the recombinant clone, the plasmid carrying the rAd.RheoIL12 genome is grown in and purified from XL10-Gold cells, digested off the plasmid backbone and packaged by transfection into HEK 293 cells or CHO cells or other suitable cell lines.

Purification of the vector to enhance the concentration can be accomplished by any suitable method, such as by density gradient purification (e.g., cesium chloride (CsCl)) or by chromatography techniques (e.g., column or batch chromatography). For example, the vector of the invention can be subjected to two or three CsCl density gradient purification steps. The vector, e.g., a replication-deficient adenoviral vector, is desirably purified from cells infected with the replication-deficient adenoviral vector using a method that comprises lysing cells infected with adenovirus, applying the lysate to a chromatography resin, eluting the adenovirus from the chromatography resin, and collecting a fraction containing adenovirus.

In a particular embodiment, the resulting primary viral stock is amplified by re-infection of HEK 293 cells or CHO cells or other suitable cell lines and is purified by CsCl density-gradient centrifugation or other suitable purification methods.

In one embodiment the IL-12 gene is a wild-type gene sequence. In another embodiment, the IL-12 gene is a modified gene sequence, e.g., a chimeric sequence or a sequence that has been modified to use preferred codons.

In one embodiment, the IL-12 gene is the human wild type sequence. In another embodiment, the sequence is at least 85% identical to wild type human sequence, e.g., at least 90%, 95%, or 99% identical to wild type human sequence. See e.g., SEQ ID NO: 3 and 4. In a further embodiment, the gene sequence encodes the human polypeptide. In another embodiment, the gene encodes a polypeptide that is at least 85% identical to wild type human polypeptide e.g., at least 90%, 95%, or 99% identical to wild type human polypeptide. See e.g., SEQ ID NO: 7 and 8.

In one embodiment, the IL-12 gene is the wild type mouse IL-12 sequence. In another embodiment, the sequence is at least 85% identical to wild type mouse IL-12, e.g., at least 90%, 95%, or 99% identical to wild type mouse IL-12. See e.g., SEQ ID NO: 1 and 2. In a further embodiment, the IL-12 gene sequence encodes the mouse IL-12 polypeptide. In another embodiment, the gene encodes a polypeptide that is at least 85% identical to wild type mouse IL-12, e.g., at least 90%, 95%, or 99% identical to wild type mouse IL-12. See e.g., SEQ ID NO: 5 and 6.

### Immune Modulators

An “immune modulator” is a type of drug (large or small molecule, including but not limited to antibodies (immunoglobulins) and other proteins), vaccine or cell therapy which induces, amplifies, attenuates or prevents change in the immune system cells, such as T cells, and some cancer cells. Non-limiting examples of immune modulators are shown in Table 10. Immune modulators may be used to treat cancer; alone or in conjuction with other compounds.

TABLE 10
Immune Modulators by Target Type

| Category | Target | Examples (non-inclusive) |
|---|---|---|
| Immune Checkpoint | PD-1 inhibitor | cemiplimab-rwlc |
| Inhibitors | | nivolumab |
| | | pembrolizumab |
| | | pidilizumab |
| | | spartalizumab |
| | | AMP-224 |
| | | AUNP-12 |
| | | BGB-A317 |
| | | MEDI-0680 |
| | | STI-A1110 |
| | PD-L1 inhibitor | atezolizumab |
| | | avelumab |
| | | durvalumab |
| | | BMS-936559 |
| | | CK-301 |
| | | KD033 |
| | Negative checkpoint | |
| | regulator | |
| | CTLA-4 inhibitor | ipilumimab |
| | | tremelimumab |
| | VISTA | anti-VISTA or VISTA-Fc fusion |
| | | protein |
| | Co-inhibitory receptor | |
| | targets | |
| | Tim-3 inhibitor (T cell | CA-327 |
| | exhaustion) | |
| | Tim-3 ligands | |
| | Galectin 9 | CNC225 |
| | HMGB1 | VB4-845 (?) |
| | phosphatidyl serine | |
| | CECAM-1 | |
| | LAG-3 | IMP321 |
| | TIGIT | BMS986207 |
| | CD25 (IL2RA) | daclizumab |
| | Toll-like Receptors (TLR) | |
| | TLR2 blockade | |
| | Intrinsic Pathways | |
| | IDO1 | indoximod |
| | TDO | CRD1152 |
| | Ectonucleotidases | |
| | CD39 | OREG-103/BY40 |
| | CD73 | BMS-986179 |
| | B7-H3 | MGC018 |
| | Bispecifics | Beta-TRAP (TGF-beta & PD-L1) |
| | | BMS-936559 (PD-L1 & B7.1) |
| Co-Stimulatory receptor | Tumor necrosis factor superfamily members | |
| targets including agonist | GITR | MEDI1873 |
| antibodies | | INCAGN01876 |
| | GITR ligand | |
| | CD27 | varlimumab |
| | CD137 (4-1BB) | urelumab |
| | CD137L (4-BBL) | |
| | OX40 (CD134) | 19B12 |
| | CD40 | MEDI6469 |
| | | dacetuzumab |
| | | ICP-870,893 |
| | Toll-like receptor (TLR) | |
| | agonists | |
| | TLR8 | VTX-2337 |
| | CD28 superfamily costimulatory molecules | |
| | BTLA | |
| | ICOS | GSK3359609 |
| Tumor-antigen-specific | EGFR | cetuximab |
| | Bispecific T cell engager | |
| | (BiTE) | |
| | CD19 | blinatumomab |
| | CD3 and EpCAM | catumaxomab |
| | CD3 and Her2/neu | ertumaxomab |
| | CD20 | |
| Immunomodulators | CD38 | daratumumab |
| | | isatuximab |
| | NK cell modulator | |
| | Killer inhibitory receptor | lirilumab |
| | (KIR) | |
| | SLAMF-7 (CSI) | elotuzumab |
| | CD96 | |
| | DNAM-1 (CD226) | |
| | NKG2A or NKG2D | IPH2201 |
| | MGN-3 | arabinoxylan |
| Viral receptor-related | Nectin-1 (CD111, PLRV1) | |
| cell adhesion molecules | Nectin-2 | |
| Vaccines | Dendritic cell vaccine | |
| | Tumor-associated peptide (TUMAP) vaccine | |
| | Oncofetal antigen vaccine | |
| | Autologous tumor cell | |
| | lysate vaccine | |
| | Viral vaccines | HPV-16 peptide |
| | | E6 |
| | | E7 |
| | Immunostimulant | TLR9 and STING ligands |
| | adjuvants | |
| | Agonists and Nonagonists for LRS, C-type lectins, and | |
| | stimulators of IFN-γ | |
| Cell Therapy | Lymphokine-activated | |
| | killer (LAK) cells | |
| | Tumor infiltrating | |
| | lymphocytes (TIL) | |
| | Cytokine-activated killer | |
| | cells | |
| | CAR-T | |
| | TCR | |

An immune modulator is for example, a immune checkpoint inhibitor, a vaccine, a molecule that stimulates T cells and/or NK cells, a cytokine, an antigen specific binder, a T cell, a NK cell, a cell expressing an introduced chimeric antigen receptor or a cell expressing an introduced T-cell receptor. Other relevant immune modulators include a chemotherapy or a radiation.

An “immune checkpoint inhibitor” is a type of drug (large or small molecule, including but not limited to antibodies (immunoglobulins) and other proteins) which block certain proteins made by some types of immune system cells, such as T cells, and some cancer cells. These proteins help keep immune responses in check and limit or prevent T cells from killing cancer cells. When these proteins are blocked, the molecular “brakes” on the immune system are released and T cells can better (i.e., more effectively) kill cancer cells. Examples of checkpoint proteins found on T cells or cancer cells include PD-1/PD-L1 and CTLA-4/B7-1/B7-2. Immune checkpoint inhibitors may be used to treat cancer; alone or in conjunction with other compounds.

In some of the embodiments of the methods described herein, the immune checkpoint inhibitor is for example, a PD-1 binder, a PD-L1 binder, a CTLA-4 binder, a V-domain immunoglobulin suppressor of T cell activation (VISTA) binder, a TIM-3 binder, a TIM-3 ligand binder, a LAG-3 binder, a T-cell immunoreceptor with Ig and ITIM domains (TIGIT) binder, a B- and T-cell attenuator (BTLA) binder, a B7-H3 binder, a TGFbeta and PD-L1 bispecific binder or a PD-L1 and B7.1 bispecific binder.

In some embodiments, the PD-1 binder is an antibody that specifically binds PD-1. In some embodiments, the PD-1 binder is an antagonist. In some embodiments, the antibody that binds PD-1 is pembrolizumab (KEYTRUDA, MK-3475; CAS #1374853-91-4) developed by Merck, pidilizumab (CT-011; CAS #1036730-42-3) developed by Curetech Ltd., nivolumab (OPDIVO, BMS-936558, MDX-1106; CAS #946414-94-4) developed by Bristol Myer Squibb, MEDI0680 (AMP-514); developed by AstraZenenca/Medlmmune, cemiplimab-rwlc (REGN2810, LIBTAYO®; CAS #1801342-60-8) developed by Regeneron Pharmaceuticals, BGB-A317 developed by BeiGene Ltd., spartalizumab (PDR-001; CAS #1935694-88-4) developed by Novartis, or STI-A1110 developed by Sorrento Therapeutics. In some embodiments, the antibody that binds PD-1 is described in PCT Publication WO2014/179664, for example, an antibody identified as APE2058, APE1922, APE1923, APE1924, APE 1950, or APE 1963 developed by Anaptysbio, or an antibody containing the CDR regions of any of these antibodies. In other embodiments, the PD-1 binder is a fusion protein that includes the extracellular domain of PD-L1 or PD-L2, for example, AMP-224 (AstraZeneca/Medlmmune). In other embodiments, the PD-1 binder is a peptide inhibitor, for example, AUNP-12 developed by Aurigene.

| Nivolumab heavy chain sequence: | |
|---|---|
| (SEQ ID NO: 10) | |
| QVQLVESGGG VVQPGRSLRL DCKASGITFS NSGMHWVRQA PGKGLEWVAV | 50 |
| IWYDGSKRYY ADSVKGRFTI SRDNSKNTLF LQMNSLRAED TAVYYCATND | 100 |
| DYWGQGTLVT VSSASTKGPS VFPLAPCSRS TSESTAALGC LVKDYFPEPV | 150 |
| TVSWNSGALT SGVHTFPAVL QSSGLYSLSS VVTVPSSSLG TKTYTCNVDH | 200 |
| KPSNTKVDKR VESKYGPPCP PCPAPEFLGG PSVFLFPPKP KDTLMISRTP | 250 |
| EVTCVVVDVS QEDPEVQFNW YVDGVEVHNA KTKPREEQFN STYRVVSVLT | 300 |
| VLHQDWLNGK EYKCKVSNKG LPSSIEKTIS KAKGQPREPQ VYTLPPSQEE | 350 |
| MTKNQVSLTC LVKGFYPSDI AVEWESNGQP ENNYKTTPPV LDSDGSFFLY | 400 |
| SRLTVDKSRW QEGNVFSCSV MHEALHNHYT QKSLSLSLGK | 440 |
| Nivolumab light chain sequence: | |
| (SEQ ID NO: 11) | |
| EIVLTQSPAT LSLSPGERAT LSCRASQSVS SYLAWYQQKP GQAPRLLIYD | 50 |
| ASNRATGIPA RFSGSGSGTD FTLTISSLEP EDFAVYYCQQ SSNWPRTFGQ | 100 |
| GTKVEIKRTV AAPSVFIFPP SDEQLKSGTA SVVCLLNNFY PREAKVQWKV | 150 |
| DNALQSGNSQ ESVTEQDSKD STYSLSSTLT LSKADYEKHK VYACEVTHQG | 200 |
| LSSPVTKSFN RGEC | |

See, WHO Drug Information, “International Nonproprietary Names for Pharmaceutical Substances (INN)”, Vol. 26, No. 2, 2012.

In some embodiments, the PD-L1 binder is an antibody that specifically binds PD-L1. In some embodiments, the PD-L1 binder is an antagonist. In some embodiments, the antibody that binds PD-L1 is atezolizumab (RG7446, MPDL3280A; Tecentriq; CAS #1380723-44-3) developed by Genentech, durvalumab (MEDI4736, IMFINZI®; CAS #1428935-60-7) developed by AstraZeneca/Medlmmune, BMS-936559 (MDX-1105) developed by Bristol Myers Squibb, avelumab (MSB0010718C; Merck KGaA; Bavencio; CAS #1537032-82-8), KD033 (Kadmon), the antibody portion of KD033, STI-A 1014 (Sorrento Therapeutics) or CK-301 (Checkpoint Therapeutics). In some embodiments, the antibody that binds PD-L1 is described in PCT Publication WO 2014/055897, for example, Ab-14, Ab-16, Ab-30, Ab-31, Ab-42, Ab-50, Ab-52, or Ab-55, or an antibody that contains the CDR regions of any of these antibodies.

In some embodiments, the CTLA-4 binder is an antibody that specifically binds CTLA-4. In some embodiments, the CTLA-4 binder is an antagonist. In some embodiments, the antibody that binds CTLA-4 is ipilimumab (YERVOY) developed by Bristol Myer Squibb or tremelimumab (CP-675,206) developed by MedImmune/AtraZenica then Pfizer. In some embodiments, the CTLA-4 binder is an antagonistic CTLA-4 fusion protein or soluble CTLA-4 receptor, for example, KAHR-102 developed by Kahr Medical Ltd.

In some embodiments, the 4-1BB (CD137) binder is a binding molecule, such as an anticalin. In some embodiments, the 41-BB binder is an agonist. In some embodiments, the anticalin is PRS-343 (Pieris AG). In some embodiments, the 4-1BB binder is an agonistic antibody that specifically binds 4-1BB. In some embodiments, antibody that binds 4-1BB is PF-2566 (PF-05082566) developed by Pfizer or urelumab (BMS-663513) developed by Bristol Myer Squibb.

In some embodiments, the LAG3 binder is an antibody that specifically binds LAG3. In some embodiments, the LAG3 binder is an antagonist. In some embodiments, the antibody that binds LAG3 is IMP701 developed by Prima BioMed, IMP731 developed by Prima BioMed/GlaxoSmithKline, BMS-986016 developed by Bristol Myer Squibb, LAG525 developed by Novartis, and GSK2831781 developed by Glaxo SmithKline. In some embodiments, the LAG-3 antagonist includes a soluble LAG-3 receptor, for example, IP321 developed by Prima BioMed.

In some embodiments, the KIR binder is an antibody that specifically binds KTR. In some embodiments, the KIR binder is an antagonist. In some embodiments, the antibody that binds KIR is lirilumab developed by Bristol Myer Squibb/Innate Pharma.

In some embodiments, a combination of controlled expression of IL-12 with a check point inhibitor, such as but not limited to, a PD-1-specific antibody (e.g., nivolumab) provides improved cancer treatment, such as but not limited to brain cancer (e.g., gliomas/glioblastomas) wherein IL-12 provides therapeutically effective recruitment and infiltration of T cells (such as killer T-cells) into the tumor while the check point inhibitor (e.g., anti-PD-1 antibody) provides for enhanced and/or improved immune cell function and activity within the tumor (i.e., improved anti-tumor immune cell activity).

In some embodiments, in conjunction with administration of a checkpoint inhibitor, methods of the invention also comprise administration of an adenovirus capable of ligand-inducible gene switch controlled-expression of IL-12, wherein the adenovirus is administered intratumorally or near (e.g., adjacent) to a tumor.

In some embodiments, in conjunction with administration of a checkpoint inhibitor, methods of the invention also comprise administration of an adenovirus capable of ligand-inducible gene switch controlled-expression of IL-12, wherein the adenovirus is administered intratumorally or near (e.g., adjacent) to a tumor via stereotactic delivery.

### Methods of Treatment

In various aspects the invention provides method of preventing, delaying the progression of, treating, alleviating a symptom of, or otherwise ameliorating cancer in a subject by administering a therapeutically effective amount of an Ad-RTS-hIL12 viral vector described herein to a subject in need thereof.

The therapeutic methods of the invention involve in vivo introduction of the polynucleotides, e.g., Ad-RTS-hIL12, into the subject. The polynucleotides may be introduced into the subject systemically or locally. For example, the polynucleotides are introduced intratumorally, at the site of the tumor, or to a lymph node associated with the tumor).

An effective amount of an Ad-RTS-hIL12 viral vector is a unit dose of about 1×10¹¹, 2×10¹¹, 3×10¹¹, 4×10¹¹, 5×10¹¹, 6×10¹¹, 7×10¹¹, 8×10¹¹, 9×10¹¹, or 1×10¹², or 2×10¹² viral particles (vp). Preferably, the viral vector is administered at a unit dose of 2×10¹¹ vp.

In some cases, the vector may be delivered by injection. In some cases, direct administration to the tumor, tumor site or lymph node includes injection of a liquid pharmaceutical composition via syringe. In another example, direct administration may involve injection via a cannula or other suitable instrument for delivery for a vector. In other examples, direct administration may comprise an implant further comprising a suitable vector for delivery of transgenes such as IL-12. In some cases the implant may be either directly implanted in or near the tumor.

The Ad-RTS-hIL12 viral vector is administered as a single administration or multiple administration, e.g., two, three, four or more administrations.

Cancers that can be treated according to the methods of the invention include a primary, progressive, metastatic or recurrent tumor. Preferably, the tumor is a solid tumors. Cancers include for example, tumors of the central nervous system, a glioma tumor, renal cancer tumor, an ovarian cancer tumor, a head and neck cancer tumor, a liver cancer tumor, a pancreatic cancer tumor, a gastric cancer tumor, an esophageal cancer tumor, a bladder cancer tumor, a ureter cancer tumor, a renal pelvis cancer tumor, a urothelial cell cancer tumor, a urogenital cancer tumor, a cervical cancer tumor, a endometrial cancer tumor, a penile cancer tumor, a thyroid cancer tumor, or a prostate cancer tumor, a breast cancer tumor, a melanoma tumor, a glioma tumor, a colon cancer tumor, a lung cancer tumor, a sarcoma cancer tumor, or a squamous cell tumor, or a prostate cancer tumor.

Tumor of the central nervous system, include for example a chordoma, a craniopharyngioma, a gangliocytoma, a glomus jugulare, a meningioma, a pineocytoma, a pineoblastoma, a pituitary adenoma, a glioma, a astrocytoma, a pilocytic astrocytoma, a “diffuse” astrocytoma, a anaplastic astrocytoma, a ependymoma, a anaplastic ependymoma, a glioblastoma multiforme (GBM), a medulloblatoma, a oligodendroglioma, a pure oligodendroglioma, a anaplastic oligodendroglioma, a anaplastic oliogoastrocytoma ganglioglioma, a acoustic neuroma (schwannoma), a vestibular schwannoma, a brain metastases, a choroid plexus carcinoma, a embryonal tumor, a germ cell tumor, a dysembryoplastic neuroepithelial tumor (DNETs), a choriocarcinoma, teratoma, a Yolk sac tumor (endodermal sinus tumor), a primary CNS lymphoma, a hemangioblastoma, a rhabdoid tumor, a glioma, a adenoma, a blastoma, a carcinoma, a sarcoma, a pineal tumor, a medulloblastoma, a medulloepithelioma, a atypical teratoid/rhabdoid tumor (ATRT), a pilocytic astrocytoma, a subependymal giant cell astrocytoma (SEGAs), a diffuse astrocytoma, a pleomorphic xanthoastrocytoma (PXAs), a optical glioma, a brain stem glioma, a focal brain stem glioma, diffuse midline glioma, a diffuse intrinsic pontine glioma (DIPGs), a midline tumor, a ganglioglioma, a craniopharyngioma, a pineal region tumor, a glioblastoma, a anaplastic astrocytoma, a embryonal tumor with multilayered rosettes, a primitive neuroectodermal tumor (PNETs), a pineoblastoma, a germinoma, a choroid plexus papilloma, a choroid plexus carcinoma, a acoustic neuroma, a neuroblastoma, a pituitary tumor, a high grade glioma, a medulloblastoma (MB), a neuroblastoma (NB), a Ewing sarcoma (EWS) or a osteosarcoma.

In one embodiment, methods of the present invention are used to treat brain cancer, such as but not limited to, malignant gliomas, primary glioblastoma, recurrent glioblastoma, progressive glioblastoma, or diffuse intrinsic pontine glioma (DIPG) and diffuse midline glioma tumors (e.g., in the thalamus, brainstem or spinal cord).

In some aspects, the methods of the present invention can be used to treat a cancer metastatic to the brain or elsewhere to the central nervous system (e.g., leptomeninges or spinal cord).

In other aspects, the methods of the present invention can be used to treat the a recurrent glioblastoma, progressive glioblastoma, or a malignant glioma.

Expression of polypeptide (e.g. IL-12) by the polynucleotide is induced by administration of a ligand as described herein to the subject.

The ligand may be administered by any suitable method, either systemically (e.g., orally, intravenously) or locally (e.g., intraperitoneally, intrathecally, intraventricularly, direct injection into the tissue or organ where the disease or disorder is occurring). Preferably, the ligand is administered orally.

The ligand is administered at a unit daily dose of about 1 mg to about 120 mg. For example, the ligand is administered at unit daily dose of about 5, 10, 15, 20, 25, 30, 35, 40, 50, 60, 70, 80, 90, 100 or 120 mg. In some embodiments the ligand is administered at a unit daily dose of about 5 mg, 10 mg, 15 mg, or 20 mg.

The ligand is administered once a day, twice a day or every other day.

Optionally, the subject is administered one or more immune modulators as described herein. The immune modulator is administered orally or parentally. For example, the immune modulator is administered intravenously. The immune modulator is administered at a dose known in the art for the particular immune modulator. For example, the immune modulator is administered at an FDA approved dose.

In preferred methods, the immune modulator is a checkpoint inhibitor such as a PD-1 binder. For example the PD-1 binder is a PD-1 antibody.

The PD-1 antibody is nivolumab (MDX 1106) and is administered at doses of about 0.5 mg/kg to about 7 mg/kg. For example, the nivolumab is administered at a dose of about 0.5 mg/kg, 1 mg/kg, 3 mg/kg, 3 mg/kg, 4 mg/kg, 5 mg/kg, 6 mg/kg, 7 mg/kg or more.

Alternatively, the nivolumab is administered at a flat dose of about between 200 mg and 500 mg. For example the flat dose is 240 mg or 480 mg.

The PDl-1 antibody is cemiplimab-rwlc (REGN-2810) and is administered at a dose of about 0.5 mg/kg to about 6 mg/kg. For example, the cemiplimab-rwlc is administered at a dose of about 0.5 mg/kg, 1 mg/kg, 2 mg/kg, 3 mg/kg, 4 mg/kg, 5 mg/kg, 6 mg/kg, or more. In further embodiments, the method optionally includes administered the subject a corticosteroid such as for example, dexamethasone. In some aspects the corticosteroid is administered during the administration of the ligand. The cumulative dose of corticosteroid during the administration of ligand is less than or equal to about 5 mg, 10 mg, 15 mg, 20 mg, 25 mg or 30 mg. Preferably the cumulative dose is less than or equal 20 mg.

The corticosteroid is administered orally or parentally. For example, the corticosteroid is administered intravenously.

Optionally, a blood vessel growth inhibitor is administered to the subject. For example, blood vessel growth inhibitor is bevacizumab. In some embodiments, bevacizumab is administered at a dose of 10 mg/kg body weight.

The term “subject,” or “individual” or “patient” as used herein in reference to individuals having a disease or disorder or are suspected of having a disease or disorder, and the like. Subject, individual or patent may be used interchangeably in the disclosure and encompass mammals and non-mammals. The subject is a pediatric patient or an adult patient.

Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In some aspects of the methods and compositions provided herein, the mammal is a human.

The subject has never previously been administered with corticosteroid. Alternatively, the subject has been previously administered a corticosteroid. For example, the has not previously been administered a corticosteroid within 4 weeks prior to the administration of the ligand. Alternatively, the subject has been administered a corticosteroid within 4 weeks prior to the administration of the ligand.

### Dosing Regimens

The invention provides dosing regimens for treating a subject having cancer with an Ad-RTS-hIL12 vector, a ligand (e.g. veledimex) and optionally an immune modulator. (“therapeutic compounds(s)”) The dosage amounts of the (“therapeutic compounds(s)” are described herein supra.

The initial dose of the vector and the initial dose of the ligand is administered concurrently or sequentially. For example, the initial dose of the ligand is administered at a period of time after the initial dose of the vector. Alternatively, initial dose of the ligand is administered at a period of time prior to the initial dose of the vector. In some embodiments the initial dose of the ligand is administered at about 1, 2, 3, 4, 5, 6, 7, 8, 9, 10, 11 or 12 hours prior to the administration of the vector. In some embodiments one or more subsequent doses of the ligand are administered once daily after the administration of the initial dose of the ligand. In other embodiments the one or more subsequent doses of the ligand are administered once daily for 3-28 days after the administration of the initial dose of the ligand. For example, daily subsequent doses of the ligand are administered for 3, 4, 5, 6, 7, 8, 9, 10, 11, 12, 13, 14, 15, 16, 17, 18, 19, 20, 21, 22, 23, 24, 25, 26, 27, 28 or more days after the after the administration of the initial dose of the ligand. Preferably, the ligand is administered daily for 14 days after the after the administration of the initial dose of the ligand. In some embodiments, a corticosteroid is further administered to the subject during the treatment period of the ligand.

The a blood vessel growth inhibitor is administered prior to the treatment period of the ligand. For example, 1, 2, 3, 4, 5, 6, or more doses of the blood vessel growth inhibitor is administered prior to the treatment period of the ligand. Preferably is administered 1, 2 or 3 doses of the blood vessel growth inhibitor are administered prior to the treatment period of the ligand.

The initial dose of the vector and the initial dose of the immune modulator is administered concurrently or sequentially. For example, the initial dose of the vector is administered at a period of time after the initial dose of the immune modulator. Alternatively, initial dose of the vector is administered at a period of time before to the initial dose of the immune modulator. In some embodiments the initial dose of the immune modulator is administered at about 1, 2, 3, 4, 5, 6, 7 or more days prior to the administration of the vector. In some embodiments one or more subsequent doses of the immune modulator are administered after the administration of the initial dose of the vector. For example, one or more subsequent doses of the immune modulator are administered within 7 to 28 days after the administration of the vector. In some. one or more subsequent doses of the immune modulator are administered embodiments at least 7, 8, 9, 10, 11, 12, 13, 14, 15, 16, 17, 18, 19, 20, 21, 22, 23, 24, 25, 26, 27, 28 or more days after administration of the vector. Preferably. one of the subsequent doses of the immune modulator are administered embodiments at 15 days after administration of the vector.

In other embodiments, subsequent doses of the immune modulator are administered once every one, two, three or four weeks after the first subsequent dose of the immune modulator. Preferably, subsequent doses of the immune modulator are administered once every two week or once every four weeks after the first subsequent dose of the immune modulator.

### Pharmaceutical Compositions

The viral vectors, ligands, immune modulators and corticosteroid described herein (also referred to herein as “therapeutic compound(s)”), can be incorporated into pharmaceutical compositions suitable for administration. Such compositions typically include the therapeutic compound(s) and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Suitable examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the disclosure is formulated to be compatible with its intended route of administration. Examples of routes of administration include parenteral, (e.g., intravenous, intradermal, subcutaneous) oral (including, inhalation), topical; (i.e., transdermal), transmucosal, or rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some embodiments, it will be desirable to include isotonic agents, for example, sugars, polyalcohols such as manitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the therapeutic compound(s) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the therapeutic compound(s) are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The therapeutic compound(s) can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of therapeutic compound(s) calculated to produce the desired therapeutic effect in association with the required pharmaceutical carrier. The specification for the dosage unit forms of the disclosure are dictated by and directly dependent on the unique characteristics of the therapeutic compound(s) and the particular therapeutic effect to be achieved, and the limitations inherent in the art of compounding such a therapeutic compound(s) for the treatment of individuals.

The pharmaceutical compositions can be included in a container, pack, or dispenser together with instructions for administration.

### Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of; cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The term “isolated” for the purposes of the invention designates a biological material (cell, nucleic acid or protein) that has been removed from its original environment (the environment in which it is naturally present). For example, a polynucleotide present in the natural state in a plant or an animal is not isolated, however the same polynucleotide separated from the adjacent nucleic acids in which it is naturally present, is considered “isolated.”

The term “purified,” as applied to biological materials does not require the material to be present in a form exhibiting absolute purity, exclusive of the presence of other compounds. It is rather a relative definition.

“Nucleic acid,” “nucleic acid molecule,” “oligonucleotide,” “nucleotide,” and “polynucleotide” are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxy cytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA.

The term “fragment,” as applied to polynucleotide sequences, refers to a nucleotide sequence of reduced length relative to the reference nucleic acid and comprising, over the common portion, a nucleotide sequence identical to the reference nucleic acid. Such a nucleic acid fragment according to the invention may be, where appropriate, included in a larger polynucleotide of which it is a constituent. Such fragments comprise, or alternatively consist of, oligonucleotides ranging in length from at least 6, 8, 9, 10, 12, 15, 18, 20, 21, 22, 23, 24, 25, 30, 39, 40, 42, 45, 48, 50, 51, 54, 57, 60, 63, 66, 70, 75, 78, 80, 90, 100, 105, 120, 135, 150, 200, 300, 500, 720, 900, 1000, 1500, 2000, 3000, 4000, 5000, or more consecutive nucleotides of a nucleic acid according to the invention.

As used herein, an “isolated nucleic acid fragment” refers to a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

A “gene” refers to a polynucleotide comprising nucleotides that encode a functional molecule, including functional molecules produced by transcription only (e.g., a bioactive RNA species) or by transcription and translation (e.g., a polypeptide). The term “gene” encompasses cDNA and genomic DNA nucleic acids. “Gene” also refers to a nucleic acid fragment that expresses a specific RNA, protein or polypeptide, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and/or coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A chimeric gene may comprise coding sequences derived from different sources and/or regulatory sequences derived from different sources. “Endogenous gene” refers to a native gene in its natural location in the genome of an organism, A “foreign” gene or “heterologous” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure. For example, the interleukin-12 (IL-12) gene encodes the EL-12 protein. IL-12 is a heterodimer of a 35-kD subunit (p35) and a 40-kD subunit (p40) linked through a disulfide linkage to make fully functional !L-12p70. The IL-12 gene encodes both the p35 and p40 subunits.

“Heterologous DNA” refers to DNA not naturally located in the cell, or in a chromosomal site of the cell. The heterologous DNA may include a gene foreign to the cell.

The term “genome” includes chromosomal as well as mitochondrial, chloroplast and viral DNA or RNA. The term “probe” refers to a single-stranded nucleic acid molecule that can base pair with a complementary single stranded target nucleic acid to form a double-stranded molecule.

As used herein, the term “oligonucleotide” refers to a short nucleic acid that is hybridizable to a genomic DNA molecule, a cDNA molecule, a plasmid DNA or an mR A molecule. Oligonucleotides can be labeled, e.g., with P-nucleotides or nucleotides to which a label, such as biotin, has been covalently conjugated. A labeled oligonucleotide can be used as a probe to detect the presence of a nucleic acid. Oligonucleotides (one or both of which may be labeled) can be used as PCR primers, either for cloning full length or a fragment of a nucleic acid, for DNA sequencing, or to detect the presence of a nucleic acid. An oligonucleotide can also be used to form a triple helix with a DNA molecule. Generally, oligonucleotides are prepared synthetically, preferably on a nucleic acid synthesizer. Accordingly, oligonucleotides can be prepared with non-naturally occurring phosphoester analog bonds, such as thioester bonds, etc.

A “primer” refers to an oligonucleotide that hybridizes to a target nucleic acid sequence to create a double stranded nucleic acid region that can serve as an initiation point for DNA synthesis under suitable conditions. Such primers may be used in a polymerase chain reaction or for DNA sequencing.

“Polymerase chain reaction” is abbreviated PCR and refers to an in vitro method for enzymatically amplifying specific nucleic acid sequences. PCR involves a repetitive series of temperature cycles with each cycle comprising three stages: denaturation of the template nucleic acid to separate the strands of the target molecule, annealing a single stranded PCR oligonucleotide primer to the template nucleic acid, and extension of the annealed primer(s) by DNA polymerase. PCR provides a means to detect the presence of the target molecule and, under quantitative or semi-quantitative conditions, to determine the relative amount of that target molecule within the starting pool of nucleic acids.

“Reverse transcription-polymerase chain reaction” is abbreviated RT-PCR and refers to an in vitro method for enzymatically producing a target cDNA molecule or molecules from an RNA molecule or molecules, followed by enzymatic amplification of a specific nucleic acid sequence or sequences within the target cDNA molecule or molecules as described above. RT-PCR also provides a means to detect the presence of the target molecule and, under quantitative or semi-quantitative conditions, to determine the relative amount of that target molecule within the starting pool of nucleic acids.

A DNA “coding sequence” or “coding region” refers to a double-stranded DNA sequence that encodes a polypeptide and can be transcribed and translated into a polypeptide in a cell, ex vivo, in vitro or in vivo when placed under the control of suitable regulatory sequences. “Suitable regulatory sequences” refers to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing sites, effector binding sites and stem-loop structures. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from mRNA, genomic jJNA sequences, and even synthetic DNA sequences. If the coding sequence is intended for expression in an eukaryotic cell, a polyadenylation signal and transcription termination sequence will usually be located 3′ to the coding sequence.

“Open reading frame” is abbreviated ORF and refers to a length of nucleic acid sequence, either DNA, cDNA or RNA, that comprises a translation start signal or initiation codon, such as an ATG or AUG, and a termination codon and can be potentially translated into a polypeptide sequence.

The term “head-to-head” is used herein to describe the orientation of two polynucleotide sequences in relation to each other. Two polynucleotides are positioned in a head-to-head orientation when the 5′ end of the coding strand of one polynucleotide is adjacent to the 5′ end of the coding strand of the other polynucleotide, whereby the direction of transcription of each polynucleotide proceeds away from the 5′ end of the other polynucleotide. The term “head-to-head”may be abbreviated (5′)-to-(5′) and may also be indicated by the symbols (>) or (3′<-5′5′-»3′).

The term “tail-to-tail” is used herein to describe the orientation of two polynucleotide sequences in relation to each other. Two polynucleotides are positioned in a tail-to-tail orientation when the 3′ end of the coding strand of one polynucleotide is adjacent to the 3′ end of the coding strand of the other polynucleotide, whereby the direction of transcription of each polynucleotide proceeds toward the other polynucleotide. The term “tail-to-tail” may be abbreviated (3′)-to-(3′) and may also be indicated by the symbols (→<-) or (5′-3′3′-5′).

The term “head-to-tail” is used herein to describe the orientation of two polynucleotide sequences in relation to each other. Two polynucleotides are positioned in a head-to-tail orientation when the 5′ end of the coding strand of one polynucleotide is adjacent to the 3′ end of the coding strand of the other polynucleotide, whereby the direction of transcription of each polynucleotide proceeds in the same direction as that of the other polynucleotide. The term “head-to-tail” may be abbreviated (5′)-to-(3′) and may also be indicated by the symbols (->⋅-) or (5′→3′5′-3′).

The term “downstream” refers to a nucleotide sequence that is located 3′ to a reference nucleotide sequence. In particular, downstream nucleotide sequences generally relate to sequences that follow the starting point of transcription. For example, the translation initiation codon of a gene is located downstream of the start site of transcription.

The term “upstream” refers to a nucleotide sequence that is located 5′ to a reference nucleotide sequence. In particular, upstream nucleotide sequences generally relate to sequences that are located on the 5′ side of a coding sequence or starting point of transcription. For example, most promoters are located upstream of the start site of transcription.

The terms “restriction endonuclease” and “restriction enzyme” are used interchangeably and refer to an enzyme that binds and cuts within a specific nucleotide sequence within double stranded DNA.

“Homologous recombination” refers to the insertion of a foreign DNA sequence into another DNA molecule, e.g., insertion of a vector in a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

A “vector” refers to any vehicle for the cloning of and/or transfer of a nucleic acid into a host cell. A vector may be a replicon to which another DNA segment may be attached so as to bring abou the replication of the attached segment. A “replicon” refers to any genetic element (e.g., plasmid, phage, eosmid, chromosome, virus) that functions as an autonomous unit of DNA replication in vivo, i.e., capable of replication under its own control. The term “vector” includes both, viral and nonviral vehicles for introducing the nucleic acid into a cell in vitro, ex vivo or in vivo. A large number of vectors known in the art may be used to manipulate nucleic acids, incorporate response elements and promoters into genes, etc. Possible vectors include, for example, plasmids or modified viruses including, for example bacteriophages such as lambda derivatives, or plasmids such as pBR322 or pUC plasmid derivatives, or the Bluescript vector. Another example of vectors that are useful in the invention is the ULTRAVECTOR® Production System (Intrexon Corp., Blacksburg, Va.) as described in WO 2007/038276. For example, the insertion of the DN fragments corresponding to response elements and promoters into a suitable vector can be accomplished by ligating the appropriate DNA fragments into a chosen vector that has complementary cohesive termini. Alternatively, the ends of the DNA molecules may be enzymatically modified or any site may be produced by ligating nucleotide sequences (linkers) into the DNA termini. Such vectors may be engineered to contain selectable marker genes that provide for the selection of cells that have incorporated the marker into the cellular genome. Such markers allow identification and/or selection of host cells that incorporate and express the proteins encoded by the marker.

Viral vectors, and particularly retroviral vectors, have been used in a wide variety of gene delivery applications in cells, as well as living animal subjects. Viral vectors that can be used include, but are not limited to, retrovirus, adeno-associated virus, pox, baculovirus, vaccinia, herpes simplex, Epstein-Barr, adenovirus, geminivirus, and caulimovirus vectors. Non-viral vectors include plasmids, liposomes, electrically charged lipids (cytofectins), DNA-protein complexes, and biopolymers. In addition to a nucleic acid, a vector may also comprise one or more regulatory regions, and/or selectable markers useful in selecting, measuring, and monitoring nucleic acid transfer results (transfer to which tissues, duration of expression, etc.).

The term “plasmid” refers to an extra-chromosomal element often carrying a gene that is not part of the central metabolism of the cell, and usually in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

A “cloning vector” refers to a “replicon,” which is a unit length of a nucleic acid, preferably DNA, that replicates sequentially and which comprises an origin of replication, such as a plasmid, phage or cosmid, to which another nucleic acid segment may be attached so as to bring about the replication of the attached segment. Cloning vectors may be capable of replication in one cell type and expression in another (“shuttle vector”). Cloning vectors may comprise one or more sequences that can be used for selection of cells comprising the vector and/or one or more multiple cloning sites for insertion of sequences of interest.

The term “expression vector” refers to a vector, plasmid or vehicle designed to enable the expression of an inserted nucleic acid sequence. The cloned gene, i.e., the inserted nucleic acid sequence, is usually placed under the control of control elements such as a promoter, a minimal promoter, an enhancer, or the like. Initiation control regions or promoters, which are useful to drive expression of a nucleic acid in the desired host cell are numerous and familiar to those skilled in the art. Virtually any promoter capable of driving expression of these genes can be used in an expression vector, including but not limited to, viral promoters, bacterial promoters, animal promoters, mammalian promoters, synthetic promoters, constitutive promoters, tissue specific promoters, pathogenesis or disease related promoters, developmental specific promoters, inducible promoters, light regulated promoters; CYC J, HIS3, GAL1, GAL4, GAL10, ADH1, PGK, PH05, GAPDH, ADC1, TRP1, URA3, LEU2, ENO, TPI, alkaline phosphatase promoters (useful for expression in Saccharomyces); AOX1 promoter (useful for expression in Pichia); β-lactamase, lac, ara, tet, trp, IPjA, IPR, T7, tac, and trc promoters (useful for expression in Escherichia coli); light regulated-, seed specific-, pollen specific-, ovary specific-, cauliflower mosaic virus 35S, CMV 35S minimal, cassava vein mosaic virus (CsVMV), chlorophyll a/b binding protein, ribulose 1,5-bisphosphate carboxylase, shoot-specific, root specific, chitinase, stress inducible, rice tungro bacilliform virus, plant super-promoter, potato leucine aminopeptidase, nitrate reductase, mannopine synthase, nopaline synthase, ubiquitin, zein protein, and anthocyanin promoters (useful for expression in plant cells); animal and mammalian promoters known in the art including, but are not limited to, the SV40 early (SV40e) promoter region, the promoter contained in the 3′ long terminal repeat (LTR) of Rous sarcoma virus (RSV), the promoters of the EIA or major late promoter (MLP) genes of adenoviruses (Ad), the cytomegalovirus (CMV) early promoter, the herpes simplex virus (HSV) thymidine kinase (TK) promoter, a baculo virus IE1 promoter, an elongation factor 1 alpha (EF1) promoter, a phosphoglycerate kinase (PGK) promoter, a ubiquitin (Ubc) promoter, an albumin promoter, the regulatory sequences of the mouse metallothionein-L promoter and transcriptional control regions, the ubiquitous promoters (HPRT, vimentin, ct-actin, tubulin and the like), the promoters of the intermediate filaments (desmin, neurofilaments, keratin, GFAP, and the like), the promoters of therapeutic genes (of the MDR, CFTR or factor VIII type, and the like), pathogenesis or disease related-promoters, and promoters that exhibit tissue specificity and have been utilized in transgenic animals, such as the elastase I gene control region which is active in pancreatic acinar cells; insulin gene control region active in pancreatic beta cells, immunoglobulin gene control region active in lymphoid cells, mouse mammary tumor virus control region active in testicular, breast, lymphoid and mast cells; albumin gene, Apo AI and Apo All control regions—'I—active in liver, alpha-fetoprotein gene control region active in liver, alpha 1-antitrypsin gene control region active in the liver, beta-globin gene control region active in myeloid cells, myelin basic protein gene control region active in oligodendrocyte cells in the brain, myosin light chain-2 gene control region active in skeletal muscle, and gonadotropic releasing hormone gene control region active in the hypothalamus, pyruvate kinase promoter, villin promoter, promoter of the fatty acid binding intestinal protein, promoter of the smooth muscle cell a-actin, and the like. In addition, these expression sequences may be modified by addition of enhancer or regulatory sequences and the like.

The term “transfection” refers to the uptake of exogenous or heterologous RNA or DNA by a cell. A cell has been “transfected” by exogenous or heterologous RNA or DNA when such RNA or DNA has been introduced inside the cell. A cell has been “transformed” by exogenous or heterologous RNA or DNA when the transfected RNA or DNA effects a phenotypic change. The transforming RNA or DNA can be integrated (covalently linked) into chromosomal DNA making up the genome of the cell.

“Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

In addition, the recombinant vector comprising a polynucleotide according to the invention may include one or more origins for replication in the cellular hosts in which their amplification or their expression is sought, markers or selectable markers.

The term “selectable marker” refers to an identifying factor, usually an antibiotic or chemical resistance gene, that is able to be selected for based upon the marker gene's effect, i.e., resistance to an antibiotic, resistance to a herbicide, colorimetric markers, enzymes, fluorescent markers, and the like, wherein the effect is used to track the inheritance of a nucleic acid of interest and/or to identify a cell or organism that has inherited the nucleic acid of interest. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. [00291] The term “reporter gene” refers to a nucleic acid encoding an identifying factor that is able to be identified based upon the reporter gene's effect, wherein the effect is used to track the inheritance of a nucleic acid of interest, to identify a cell or organism that has inherited the nucleic acid of interest, and/or to measure gene expression induction or transcription. Examples of reporter genes known and used in the art include: luciferase (Luc), green fluorescent protein (GFP), chloramphenicol acetyltransferase (CAT), β-galactosidase (LacZ), β-glucuronidase (Gus), and the like. Selectable marker genes may also be considered reporter genes.

“Promoter” and “promoter sequence” are used interchangeably and refer to a DNA sequence capable of controlling the expression of a coding sequence or functional R A. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths may have identical promoter activity.

In any of the vectors of the present invention, the vector optionally comprises a promoter disclosed herein.

In any of the vectors of the present invention, the vector optionally comprises a tissue-specific promoter. In one embodiment, the tissue-specific promoter is a tissue specific promoter disclosed herein.

The promoter sequence is typically bounded at its 3′terminus by the transcription initiation site and extends upstream (5′ direction) to include the minimum number of bases or elements necessary to initiate transcription at levels detectable above background. Within the promoter sequence is found a transcription initiation site (conveniently defined for example, by mapping with nuclease SI), as well as protein binding domains (consensus sequences) responsible for the binding of RNA polymerase.

“Therapeutic switch promoter” (“TSP”) refers to a promoter that controls expression of a gene switch component. Gene switches and their various components are described in detail elsewhere herein. In certain embodiments a TSP is constitutive, i.e., continuously active. A consitutive TSP may be either constitutive-ubiquitous (i.e., generally functions, without the need for additional factors or regulators, in any tissue or cell) or constitutive-tissue or cell specific (i.e., generally functions, without the need for additional factors or regulators, in a specific tissue type or cell type). In certain embodiments a TSP of the invention is activated under conditions associated with a disease, disorder, or condition. In certain embodiments of the invention where two or more TSPs are involved the promoters may be a combination of constitutive and activatable promoters. As used herein, a “promoter activated under conditions associated with a disease, disorder, or condition” includes, without limitation, disease-specific promoters, promoters responsive to particular physiological, developmental, differentiation, or pathological conditions, promoters responsive to specific biological molecules, and promoters specific for a particular tissue or cell type associated with the disease, disorder, or condition, e.g. tumor tissue or malignant cells. TSPs can comprise the sequence of naturally occurring promoters, modified sequences derived from naturally occurring promoters, or synthetic sequences (e.g., insertion of a response element into a minimal promoter sequence to alter the responsiveness of the promoter).

A coding sequence is “under the control” of transcriptional and translational control sequences in a cell when RNA polymerase transcribes the coding sequence into mRNA, which is then trans-RNA spliced (if the coding sequence contains introns) and translated into the protein encoded by the coding sequence.

“Transcriptional and translational control sequences” refer to DNA regulatory sequences, such as promoters, enhancers, terminators, and the like, that provide for the expression of a coding sequence in a host cell. In eukaryotic cells, polyadenylation signals are control sequences.

The term “response element” refers to one or more cis-acting DNA elements which confer responsiveness on a promoter mediated through interaction with the DNA-binding domains of a transcription factor. This DNA element may be either palindromic (perfect or imperfect) in its sequence or composed of sequence motifs or half sites separated by a variable number of nucleotides. The half sites can be similar or identical and arranged as either direct or inverted repeats or as a single half site or multimers of adjacent half sites in tandem. The response element may comprise a minimal promoter isolated from different organisms depending upon the nature of the cell or organism into which the response element is incorporated. The DNA binding domain of the transcription factor binds, in the presence or absence of a ligand, to the DNA sequence of a response element to initiate or suppress transcription of downstream gene(s) under the regulation of this response element. Examples of DNA sequences for response elements of the natural ecdysone receptor include: RRGG/TTCANTGAC/ACYY (SEQ ID NO: 16) (see Cherbas et. al., Genes Dev. 5:120 (1991)); AGGTCAN₍ₙ₎AGGTCA, where N₍ₙ₎ can be one or more spacer nucleotides (SEQ ID NO: 17) (see DAvino et al., Mol. Cell. Endocrinol. 113:1 (1995)); and GGGTTGAATGAATTT (SEQ ID NO: 18) (see Antoniewski et al., Mol. Cell Biol. 14:4465 (1994)).

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression” as used herein refers to the transcription and stable accumulation of sense (mRNA) or antisense RNA derived from a nucleic acid or polynucleotide. Expression may also refer to translation of mRNA into a protein or polypeptide. [00302] The terms “cassette,” “expression cassette” and “gene expression cassette” refer to a segment of DNA that can be inserted into a nucleic acid or polynucleotide at specific restriction sites or by homologous recombination. The segment of DNA comprises a polynucleotide that encodes a polypeptide of interest, and the cassette and restriction sites are designed to ensure insertion of the cassette in the proper reading frame for transcription and translation. “Transformation cassette” refers to a specific vector comprising a polynucleotide that encodes a polypeptide of interest and having elements in addition to the polynucleotide that facilitate transformation of a particular host cell. Cassettes, expression cassettes, gene expression cassettes and transformation cassettes of the invention may also comprise elements that allow for enhanced expression of a polynucleotide encoding a polypeptide of interest in a host cell. These elements may include, but are not limited to: a promoter, a minimal promoter, an enhancer, a response element, a terminator sequence, a polyadenylation sequence, and the like.

For purposes of this invention, the term “gene switch” refers to the combination of a response element associated with a promoter, and a ligand-dependent transcription factor-based system which, in the presence of one or more ligands, modulates the expression of a gene into which the response element and promoter are incorporated. The term “a polynucleotide encoding a gene switch” refers to the combination of a response element associated with a promoter, and a polynucleotide encoding a ligand-dependent transcription factor-based system which, in the presence of one or more ligands, modulates the expression of a gene into which the response element and promoter are incorporated.

The therapeutic switch promoters of the invention may be any promoter that is useful for treating, ameliorating, or preventing a specific disease, disorder, or condition. Examples include, without limitation, promoters of genes that exhibit increased expression only during a specific disease, disorder, or condition and promoters of genes that exhibit increased expression under specific cell conditions (e.g., proliferation, apoptosis, change in pH, oxidation state, oxygen level). In some embodiments where the gene switch comprises more than one transcription factor sequence, the specificity of the therapeutic methods can be increased by combining a disease- or condition-specific promoter with a tissue- or cell type-specific promoter to limit the tissues in which the therapeutic product is expressed. Thus, tissue- or cell type-specific promoters are encompassed within the definition of therapeutic switch promoter.

As an example of disease-specific promoters, useful promoters for treating cancer include the promoters of oncogenes. Examples of classes of oncogenes include, but are not limited to, growth factors, growth factor receptors, protein kinases, programmed cell death regulators and transcription factors. Specific examples of oncogenes include, but are not limited to, sis, erb B, erb B-2, ras, abl, myc and bcl-2 and TERT. Examples of other cancer-related genes include tumor associated antigen genes and other genes that are overexpressed in neoplastic cells (e.g., MAGE-1, carcinoembryonic antigen, tyrosinase, prostate specific antigen, prostate specific membrane antigen, p53, MUC-1, MUC-2, MUC-4, HER-2/neu, T/Tn, MART-1, gpl OO, GM2, Tn, sTn, and Thompson-Friedenreich antigen (TF)).

The source of the promoter that is inserted into the gene switch can be natural or synthetic, and the source of the promoter should not limit the scope of the invention described herein. In other words, the promoter may be directly cloned from cells, or the promoter may have been previously cloned from a different source, or the promoter may have been synthesized.

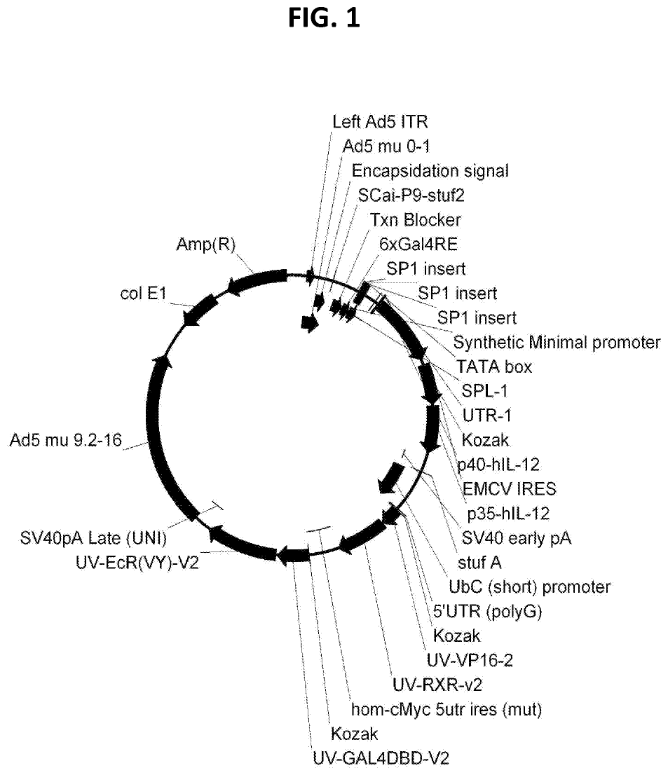

The term “ecdysone receptor-based,” with respect to a gene switch, refers to a gene switch comprising at least a functional part of a naturally occurring or synthetic ecdysone receptor ligand binding domain and which regulates gene expression in response to a ligand that binds to the ecdysone receptor ligand binding domain. Examples of ecdysone-responsive systems are described in U.S. Pat. Nos. 7,091,038 and 6,258,603. In one embodiment, the system is the RheoSwitch® Therapeutic System (RTS), which contains two fusion proteins, the DEF domains of a mutagenized ecdysone receptor (EcR) fused with a Gal4 DNA binding domain and the EF domains of a chimeric RXR fused with a VP16 transcription activation domain, expressed under a constitutive promoter as illustrated in FIG. 1.

The term “ligand-dependent transcription factor” (LDTF) refers to a transcription factor comprising one or more protein subunits, which complex can regulate gene expression driven by a transcription factor-regulated promoter. One such example is an “ecdysone receptor complex” generally refers to a heterodimeric protein complex having at least two members of the nuclear receptor family, ecdysone receptor (“EcR”) and ultraspiracle (“USP”) proteins (see Yao et al., Nature 366:476 (1993)); Yao et al., Cell 71:63 (1992)). A functional LDTF such as an EcR complex may also include additional protein(s) such as immunophilins. Additional members of the nuclear receptor family of proteins, known as transcriptional factors (such as DHR38, betaFTZ-1 or other insect homologs), may also be ligand dependent or independent partners for EcR and/or USP. A LDTFC such as an EcR complex can also be a heterodimer of EcR protein and the vertebrate homolog of ultraspiracle protein, retinoic acid-X-receptor (“RXR”) protein or a chimera of USP and RXR. The terms “LDTFC” and “EcR complex” also encompass homodimer complexes of the EcR protein or USP, as well as single polypeptides or trimers, tetramer, and other multimers serving the same function.

The terms “modulate” and “modulates” mean to induce, reduce or inhibit nucleic acid or gene expression, resulting in the respective induction, reduction or inhibition of protein or polypeptide production.

The polynucleotides or vectors according to the invention may further comprise at least one promoter suitable for driving expression of a gene in a host cell.

Enhancers that may be used in embodiments of the invention include but are not limited to: an SV40 enhancer, a cytomegalovirus (CMV) enhancer, an elongation factor 1 (EF 1) enhancer, yeast enhancers, viral gene enhancers, and the like.

Termination control regions, i.e., terminator or polyadenylation sequences, may also be derived from various genes native to the preferred hosts. Optionally, a termination site may be unnecessary, however, it is most preferred if included. In one embodiment of the invention, the termination control region may be comprised or be derived from a synthetic sequence, synthetic polyadenylation signal, an SV40 late polyadenylation signal, an SV40 polyadenylation signal, a bovine growth hormone (BGH) polyadenylation signal, viral terminator sequences, or the like.

The terms “3′ non-coding sequences” or “3′ untranslated region (UTR)” refer to DNA sequences located downstream (3′) of a coding sequence and may comprise polyadenylation [poly(A)] recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“Regulatory region” refers to a nucleic acid sequence that regulates the expression of a second nucleic acid sequence. A regulatory region may include sequences which are naturally responsible for expressing a particular nucleic acid (a homologous region) or may include sequences of a different origin that are responsible for expressing different proteins or even synthetic proteins (a heterologous region). In particular, the sequences can be sequences of prokaryotic, eukaryotic, or viral genes or derived sequences that stimulate or repress transcription of a gene in a specific or non-specific manner and in an inducible or non-inducible manner. Regulatory regions include origins of replication, RNA splice sites, promoters, enhancers, transcriptional termination sequences, and signal sequences which direct the polypeptide into the secretory pathways of the target cell.

A regulatory region from a “heterologous source” refers to a regulatory region that is not naturally associated with the expressed nucleic acid. Included among the heterologous regulatory regions are regulatory regions from a different species, regulatory regions from a different gene, hybrid regulatory sequences, and regulatory sequences which do not occur in nature, but which are designed by one having ordinary skill in the art.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. “Messenger RNA (mRNA)” refers to the RNA that is without introns and that can be translated into protein by the cell. “cDNA” refers to a double-stranded DNA that is complementary to and derived from mRNA. “Sense” RNA refers to RNA transcript that includes the mRNA and so can be translated into protein by the cell. “Antisense RNA” refers to a RNA transcript that is complementary to all or part of a target primary transcript or mRNA and that blocks the expression of a target gene. The complementarity of an antisense RNA may be with any part of the specific gene transcript, i.e., at the 5′ non-coding sequence, 3′ non-coding sequence, or the coding sequence. “Functional RNA” refers to antisense RNA, ribozyme RNA, or other RNA that is not translated yet has an effect on cellular processes.

“Polypeptide,” “peptide” and “protein” are used interchangeably and refer to a polymeric compound comprised of covalently linked amino acid residues.

An “isolated polypeptide,” “isolated peptide” or “isolated protein” refer to a polypeptide or protein that is substantially free of those compounds that are normally associated therewith in its natural state (e.g., other proteins or polypeptides, nucleic acids, carbohydrates, lipids). “Isolated” is not meant to exclude artificial or synthetic mixtures with other compounds, or the presence of impurities which do not interfere with biological activity, and which may be, for example, due to incomplete purification, addition of stabilizers, or compounding into a pharmaceutically acceptable preparation.

The term “fragment,” as applied to a polypeptide, refers to a polypeptide whose amino acid sequence is shorter than that of the reference polypeptide and which comprises, over the entire portion with these reference polypeptides, an identical amino acid sequence. Such fragments may, where appropriate, be included in a larger polypeptide of which they are a part. Such fragments of a polypeptide according to the invention may have a length of at least 2, 3, 4, 5, 6, 8, 10, 13, 14, 15, 16, 17, 18, 19, 20, 21, 22, 25, 26, 30, 35, 40, 45, 50, 100, 200, 240, or 300 or more amino acids.

A “variant” of a polypeptide or protein refers to any analogue, fragment, derivative, or mutant which is derived from a polypeptide or protein and which retains at least one biological property of the polypeptide or protein. Different variants of the polypeptide or protein may exist in nature. These variants may be allelic variations characterized by differences in the nucleotide sequences of the structural gene coding for the protein, or may involve differential splicing or post-translational modification. The skilled artisan can produce variants having single or multiple amino acid substitutions, deletions, additions, or replacements. These variants may include, inter alia: (a) variants in which one or more amino acid residues are substituted with conservative or non-conservative amino acids, (b) variants in which one or more amino acids are added to the polypeptide or protein, (c) variants in which one or more of the amino acids includes a substituent group, and (d) variants in which the polypeptide or protein is fused with another polypeptide such as serum albumin. The techniques for obtaining these variants, including genetic (suppressions, deletions, mutations, etc.), chemical, and enzymatic techniques, are known to persons having ordinary skill in the art. In one embodiment, a variant polypeptide comprises at least about 14 amino acids.

The term “homology” refers to the percent of identity between two polynucleotide or two polypeptide moieties. The correspondence between the sequence from one moiety to another can be determined by techniques known to the art. For example, homology can be determined by a direct comparison of the sequence information between two polypeptide molecules by aligning the sequence information and using readily available computer programs. Alternatively, homology can be determined by hybridization of polynucleotides under conditions that form stable duplexes between homologous regions, followed by digestion with single-stranded-specific nuclease(s) and size determination of the digested fragments.

As used herein, the term “homologous” in all its grammatical forms and spelling variations refers to the relationship between proteins that possess a “common evolutionary origin,” including proteins from superfamilies (e.g., the immunoglobulin superfamily) and homologous proteins from different species (e.g., myosin light chain, etc.) (Reeck et al., Cell 50:667 (1987)). Such proteins (and their encoding genes) have sequence homology, as reflected by their high degree of sequence similarity. However, in common usage and in the application, the term “homologous,” when modified with an adverb such as “highly,” may refer to sequence similarity and not a common evolutionary origin.

The term “corresponding to” is used herein to refer to similar or homologous sequences, whether the exact position is identical or different from the molecule to which the similarity or homology is measured. A nucleic acid or amino acid sequence alignment may include spaces. Thus, the term “corresponding to” refers to the sequence similarity, and not the numbering of the amino acid residues or nucleotide bases.

A “substantial portion” of an amino acid or nucleotide sequence comprises enough of the amino acid sequence of a polypeptide or the nucleotide sequence of a gene to putatively identify that polypeptide or gene, either by manual evaluation of the sequence by one skilled in the art, or by computer-automated sequence comparison and identification using algorithms such as BLAST (Basic Local Alignment Search Tool; Altschul et al., J. Mol. Biol. 215:403 (1993)); available at ncbi.nlm.nih.gov/BLAST/). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to specifically identify and/or isolate a nucleic acid fragment comprising the sequence.

The term “percent identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “identity” and “similarity” can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). Preferred methods to determine identity are designed to give the best match between the sequences tested. Methods to determine identity and similarity are codified in publicly available computer programs. Sequence alignments and percent identity calculations may be performed using sequence analysis software such as the MegAlign (or more recently MegAlign Pro) program of the LASERGENE bioinformatics computing suite (DNASTAR Inc., Madison, Wis.). Multiple alignment of the sequences may be performed using a Clustal method of alignment (Higgins et al., CABIOS. 5:151 1989) with the default parameters (GAP PENALTY=10, GAP LENGTH PENALTY=10). Default parameters for pairwise alignments using a Clustal method may be selected: KTUPLE 1, GAP PENALTY=3, WINDOW=5 and DIAGONALS SAVED=5.

As used herein, two or more individually operable gene regulation systems are said to be “orthogonal” when; a) modulation of each of the given systems by its respective ligand, at a chosen concentration, results in a measurable change in the magnitude of expression of the gene of that system, and b) the change is statistically significantly different than the change in expression of all other systems simultaneously operable in the cell, tissue, or organism, regardless of the simultaneity or sequentiality of the actual modulation. Preferably, modulation of each individually operable gene regulation system effects a change in gene expression at least 2-fold greater than all other operable systems in the cell, tissue, or organism, e.g., at least 5-fold, 10-fold, 100-fold, or 500-fold greater. Ideally, modulation of each of the given systems by its respective ligand at a chosen concentration results in a measurable change in the magnitude of expression of the gene of that system and no measurable change in expression of all other systems operable in the cell, tissue, or organism. In such cases the multiple inducible gene regulation system is said to be “fully orthogonal.” Useful orthogonal ligands and orthogonal receptor-based gene expression systems are described in US 2002/0110861 A1.

The term “exogenous gene” means a gene foreign to the subject, that is, a gene which is introduced into the subject through a transformation process, an unmutated version of an endogenous mutated gene or a mutated version of an endogenous unmutated gene. The method of transformation is not critical to this invention and may be any method suitable for the subject known to those in the art. Exogenous genes can be either natural or synthetic genes which are introduced into the subject in the form of DNA or RNA which may function through a DNA intermediate such as by reverse transcriptase. Such genes can be introduced into target cells, directly introduced into the subject, or indirectly introduced by the transfer of transformed cells into the subject.

The term “therapeutic product” refers to a therapeutic polypeptide or therapeutic polynucleotide which imparts a beneficial function to the host cell in which such product is expressed. Therapeutic polypeptides may include, without limitation, peptides as small as three amino acids in length, single- or multiple-chain proteins, and fusion proteins. Therapeutic polynucleotides may include, without limitation, antisense oligonucleotides, small interfering RNAs, ribozymes, and RNA external guide sequences. The therapeutic product may comprise a naturally occurring sequence, a synthetic sequence or a combination of natural and synthetic sequences.

As used herein, the terms “activating” or “activate” refer to any measurable increase in cellular activity of a gene switch, resulting in expression of a gene of interest, e.g., IL-12.

As used herein, the terms “treating” or “treatment” of a disease refer to executing a protocol, which may include administering one or more drugs or in vitro engineered cells to a mammal (human or non-human), in an effort to alleviate signs or symptoms of the disease. Thus, “treating” or “treatment” should not necessarily be construed to require complete alleviation of signs or symptoms, does not require a cure, and specifically includes protocols that have only marginal effect on the subject.

As used herein, “immune cells” include dendritic cells, macrophages, neutrophils, mast cells, eosinophils, basophils, natural killer cells and lymphocytes (e.g., B and T cells).

As used herein, the terms “MOI” or “Multiplicity of Infection” refer to the average number of adenovirus particles that infect a single cell in a specific experiment (e.g., recombinant adenovirus or control adenovirus).

As used herein, the term “binder” refers to a molecule that binds to a polypeptide or epitope of a polyeptide. A binder can be an antagonist or an agonist.

As used herein, the term “tumor” refers to all benign or malignant cell growth and proliferation either in vivo or in vitro, whether precancerous or cancerous cells and/or tissues.

As used herein, the term “binder” is a composition that binds to a target. A binder is a molecule that by attractive interactions forms a stable association with a target molecule, which may be reversible or irreversible. Attractive interactions may include for example, non-covalent interactions, which include but are not limited to electrostatic interactions, Van der Waals forces and hydrophobic effects. For example, a PD-1 binder binds to a PD-1. The binder can be an antagonist, an agonist or a co-stimulatory molecule. As used herein, the term “tumor” refers to all benign or malignant cell growth and proliferation either in vivo or in vitro, whether precancerous or cancerous cells and/or tissues.

As used herein, a “dosage regimen” or “dosing regimen” includes a treatment regimen based on a determined set of doses.

As used herein, the term “dosing”, as used herein, refers to the administration of a substance (e.g., Ad-RTS-hIL-12 and veledimex and nivolumab) to achieve a therapeutic objective (e.g., the treatment of a central nervous system tumor).

### EXAMPLES

The following working examples are illustrative and are not intended to be limiting and it will be readily understood by one of skill in the art that other embodiments may be utilized.

### Example 1 Veledimex Crosses the Blood-Brain Barrier in GL-261 Orthotopic Glioma Mice and Normal Mice

To generate orthotopic GL-261 glioma mice, a group of C57BL/6 mice received 1×105 GL-261 glioma cells via intracranial injection ˜2 mm distal to the intersection of the coronal and sagittal suture. On day 5, the animals were randomly assigned to one of the treatment groups. GL-261, a murine glioma tumor cell line, was purchased from American Type Culture Collection (Manassas, Va.).

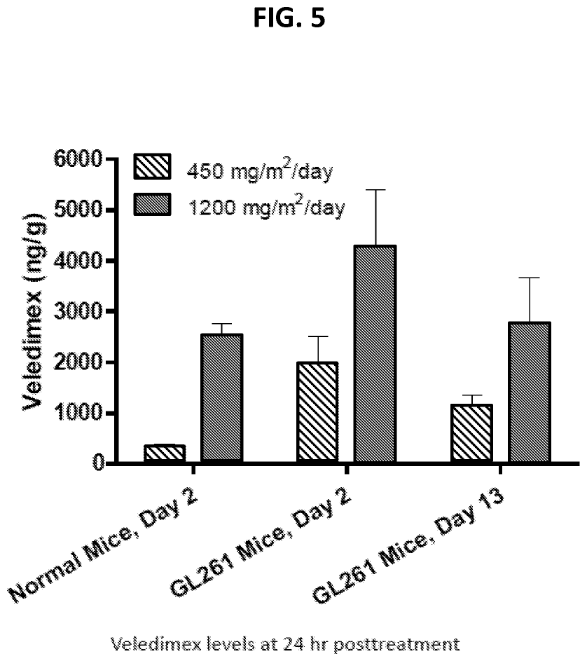

Veledimex was administered to normal C57BL/6 mice or orthotopic GL-261 glioma mice via oral gavage (PO) at 450 mg/m2/day or 1,200 mg/m²/day. Terminal blood and CSF were collected for normal C57BL/6 mice or orthotopic GL-261 glioma mice after 2 days of treatment, and for orthotopic GL-261 glioma mice after 13 days of treatment. The veledimex levels at 24 hours post-veledimex treatment were quantified and shown in FIG. 5. These data demonstrated that orally administered veledimex crosses the blood-brain barrier in both normal C57BL/6 mice and orthotopic GL-261 glioma mice at sufficient levels to warrant assessment of IL-12 expression in vivo via administration of Ad-RTS-IL-12 plus veledimex in glioma.

### Example 2—Ad-RTS-mIL-12 Plus (+) Veledimex in Combination with Anti-PD-1 Antibody Improves Survival

An orthotopic GL-261 mouse model was used to assess the effects of adenovirus expressing murine IL-12 via veledimex induced expression, as controlled via an ecdysone receptor-based expression system (i.e., referred to as “Ad-RTS-mIL-12”) (5×10⁹ viral particles (vp)) with veledimex only (10-30 mg/m²/day for 14 days) versus Ad-RTS-mIL-12 with veledimex in combination with PD-1-specific monoclonal antibody (i.e., mAb RMP1-14) (anti-PD-1 at 7.5 and 15 mg/m²).

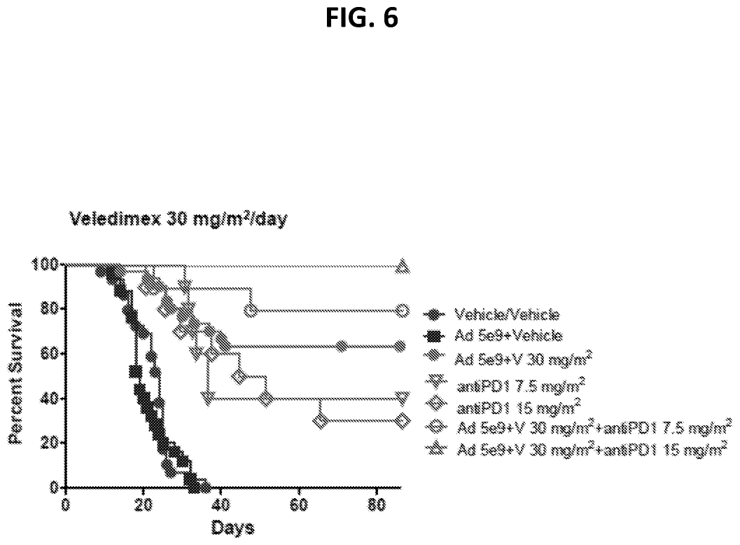

As shown in FIG. 6, all mice without treatment succumb to disease progression by Day 35. Eighty days after immunotherapy, 70-80% receiving Ad-RTS-mIL-12 plus veledimex monotherapy survived, 30-40% receiving anti-PD-1 monotherapy survived, and 100% receiving a combination of Ad-RTS-mIL-12 plus veledimex at 30 mg/m² with anti-PD-1 (anti-mouse PD-1 (CD279, clone RMP1-14, InVivoPlus, cat #BP0146, BioXCell, West Lebanon, N.H.) at 15 mg/m² survived.

There was an observed increase in tumor localized IL-12 (100 pg/mg) which was 15-times greater than that observed at peak plasma levels, 5 days after Ad-RTS-mIL-12 plus veledimex. Furthermore, the combination of Ad-RTS-mIL-12 plus veledimex with anti-PD-1 sustained peak IL-12 levels in tumors and was associated with a 100-150% increase of activated T cells in spleens compared with the minimal changes observed with either immunotherapy alone. In addition, there was a significant reduction in regulatory T cells (FoxP3+) compared with monotherapies. In conclusion, murine model studies using controlled local immunostimulation with IL-12 combined with inhibition of PD-1 demonstrated this type of therapy to be a potentially promising approach for treatment of glioma.

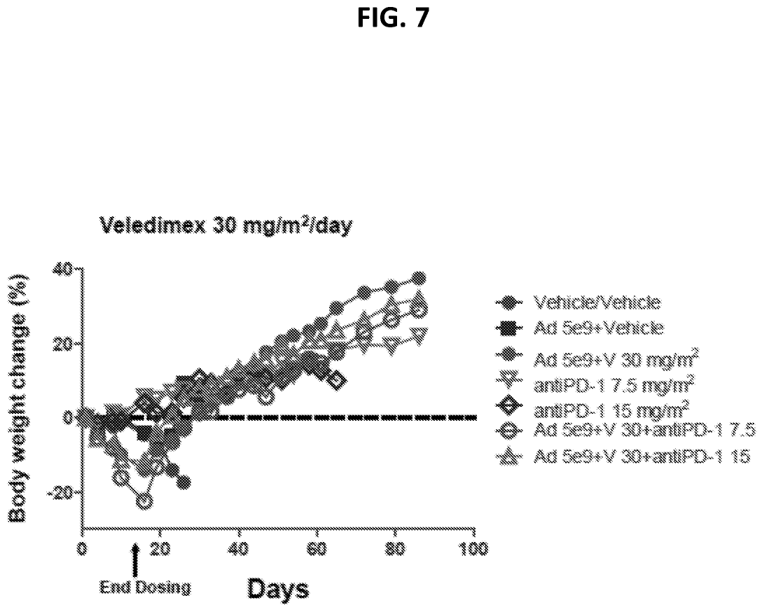

Consistent with disease progression, combination therapy of Ad-RTS-mIL-12 plus veledimex with anti-PD-1 augmented reductions in body weight change compared to Ad-RTS-mIL-12 plus veledimex monotherapy or anti-PD-1 monotherapy, as shown in FIG. 7. All groups recovered when veledimex was discontinued.

### Example 3 Effects of Ad-RTS-mIL-12 Plus (+) Veledimex in Combination with Anti-PD-1 Antibody on Local Cytokine Production

The ability of intratumoral Ad-RTS-mIL-12 at 5×10⁹ plus (+) oral veledimex at 30 mg/m2, with or without anti-PD-1, to locally produce IL-12 and stimulate IFN-γ production in the tumor in the GL-261 orthotopic glioma mouse model was explored. Tumor samples from mice in each group were collected for evaluation of IL-12 and IFN-γ levels via ELISA. As shown in FIG. 8A, there was an increase in tumor IL-12 (100 pg/mg), which was 15 times greater than that of plasma peak 5 days after Ad-RTS-mIL-12 plus veledimex. The combination of Ad-RTS-mIL-12 plus veledimex with anti-PD-1 produced sustained peak IL-12 levels in tumor. IFN-γ followed a similar trend coinciding with the peak increases of IL-12; thereby confirming that IL-12 produced by the vector was biologically active.

### Example 4—Effects of Ad-RTS-mIL-12 Plus Veledimex in Combination with Anti-PD-1 Antibody on T-Cell Activation

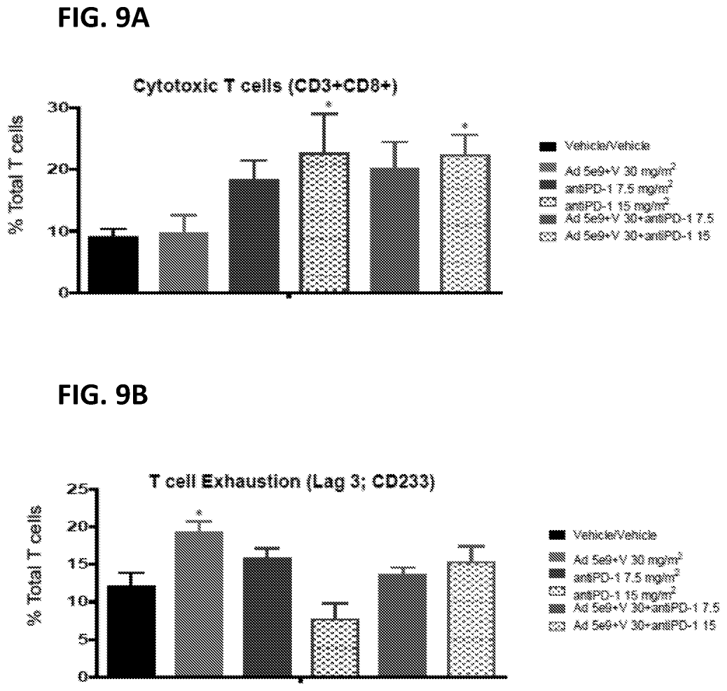

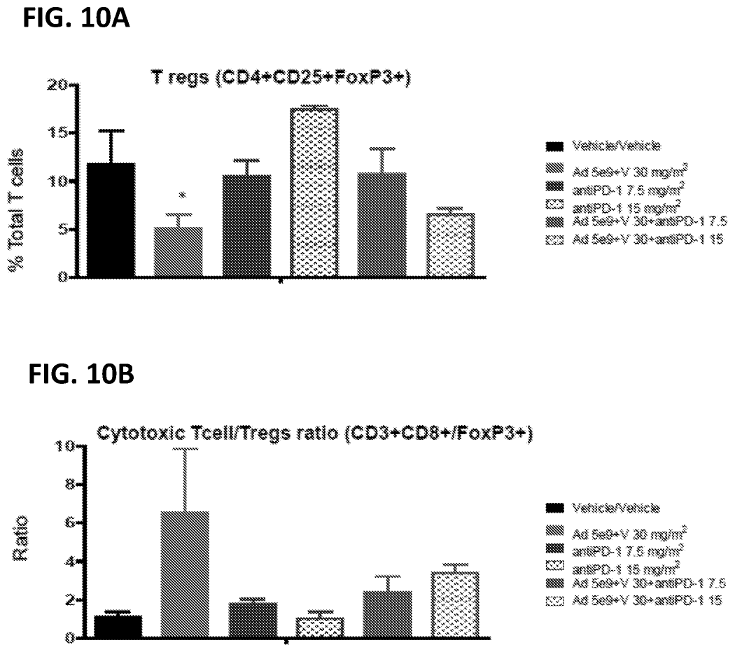

The effects of Ad-RTS-mIL-12 plus veledimex with anti-PD-1 antibody therapy on the tumor microenvironment and recruitment of effector and regulatory T cells in the GL-261 orthotopic glioma mouse model was assessed. There was an observed 100% to 150% increase of activated cytotoxic T cells (CD3⁺CD8⁺) in the spleen, compared with the minimal changes observed with either immunotherapy alone (FIG. 9A). An increase in T-cell exhaustion (LAG3*) was also observed in some treatment groups during the active dosing period (FIG. 9B). In addition, there was a significant reduction in regulatory T cells (CD4⁺CD25⁺FoxP3⁺) compared to the monotherapies (FIGS. 10A and B). In conclusion, controlled local immune-stimulation with IL-12 combined with inhibition of PD-1 is a potentially promising approach for the treatment of glioma.

### Example 5—Clinical Protocol for Ad-RTS-mIL-12 Plus Veledimex with Anti-PD-1 Antibody Therapy

The following is an example of parameters which may be used in a (human) clinical protocol to practice the invention; i.e., in the form of the administration of Ad-RTS-IL-12 plus veledimex in combination with PD-1-specific antibodies for the treatment of glioma, including but not limited to recurrent or progressive glioblastoma.

Targeted objectives are: (1) Assess safety and tolerability of intratumoral administration of adenovirus-delivery and expression of IL-12 via Ad-RTS—using varying levels (doses) of oral (PO) veledimex (small molecule activator ligand) in combination with an anti-PD-1 immunoglobulin (for example, but not limited to, nivolumab) in subjects with recurrent or progressive glioblastoma; (2) Determine optimal dose of Ad-RTS-hTL-12 plus veledimex when administered in combination with anti-PD-1 antibody (e.g., nivolumab); (3) Determine (via an investigator's assessment of response) tumor objective response rate (ORR), progression free survival (PFS), and rate of pseudo-progression (PSP) of Ad-RTS-hTL-12 plus veledimex when administered in combination with anti-PD-1 antibody (e.g., nivolumab); (4) Determine overall survival (OS) of Ad-RTS-hTL-12 plus veledimex when administered in combination with nivolumab; (5) Evaluate cellular and humoral immune responses elicited by Ad-RTS-hTL-12 plus veledimex when administered in combination with nivolumab; and, (6) Determine the veledimex pharmacokinetic (PK) profile after administration of anti-PD-1 antibody (e.g., nivolumab).

A target study population includes adult humans (subjects) with glioma, such as recurrent or progressive glioblastoma. In certain study subsets, subjects with glioblastoma have not previously been treated with inhibitors of immune checkpoint pathways (e.g., anti-PD-1, anti-PD-L1, anti-PD-L2, anti-CD137, or anti-CTLA-4 antibody) or other agents specifically targeting T cells.

Criteria for a target subject population may include: (1) male or female subject ≥18 and ≤75 years of age; (2) provisions for tumor resection, tumor biopsy, and/or samples collection; (3) histologically confirmed supratentorial glioblastoma or other World Health Organization (WHO) Grade III or IV malignant glioma from archival tissue; (4) evidence of tumor recurrence/progression by magnetic resonance imaging (MRI) according to Response Assessment in Neuro-Oncology (RANO) criteria after standard initial therapy; (5) previous standard-of-care antitumor treatment including surgery and/or biopsy and chemoradiation. Study criteria may include that subjects have recovered from the toxic effects of previous treatments, if any, as determined by a physician. Such “washout periods” from prior therapies are may be defined as follows: (1) nitrosureas, 6 weeks; (2) other cytotoxic agents, 4 weeks; (3) antiangiogenic agents, including bevacizumab, 4 weeks; (4) other cancer targeting agents, including small molecule tyrosine kinase inhibitors, 2 weeks; (5) vaccine-based therapy, 3 months; (6) able to undergo standard MRI scans with contrast agent before enrollment and after treatment; (7) Karnofsky Performance Status ≥70; (8) adequate bone marrow reserves and liver and kidney function (as assessed by the following laboratory requirements: (a) hemoglobin ≥9 g/L; (b) lymphocytes >500/mm3; (c) absolute neutrophil count ≥1500/mm3; (d) platelets ≥100,000/mm³; (e) serum creatinine ≤1.5× upper limit of normal (ULN); (f) aspartate transaminase (AST) and alanine transaminase (ALT)≤2.5×ULN for subjects with documented liver metastases, ALT and AST≤5×ULN; (g) total bilirubin <1.5×ULN; (h) International normalized ratio (INR) and activated partial thromboplastin time (aPTT) within normal institutional limits); (9) male and female subjects agree to use a highly reliable method of birth control (expected failure rate <5% per year) from initial study screening until after the last dose of study drug. Women of childbearing potential (perimenopausal women must be amenorrheic for at least 12 months to be considered of non-childbearing potential) must have a negative pregnancy test at screening; (10) normal cardiac and pulmonary function as evidenced by a normal ECG and peripheral oxygen saturation (SpO2) ≥90% by pulse oximetry.

Subject exclusion criteria may include any one or more of: (1) radiotherapy treatment within 4 weeks of starting veledimex; (2) subjects with clinically significant increased intracranial pressure (e.g., impending herniation or requirement for immediate palliative treatment) or uncontrolled seizures; (3) known immunosuppressive disease, or autoimmune conditions, and/or chronic viral infections (e.g., human immunodeficiency virus [HIV], hepatitis); (4) use of systemic antibacterial, antifungal, or antiviral medications for the treatment of acute clinically significant infection within 2 weeks of first veledimex dose. Concomitant therapy for chronic infections is not allowed. Subjects are afebrile prior to Ad-RTS-hIL-12 injection; only prophylactic antibiotic is used perioperatively, if necessary; (5) use of enzyme-inducing antiepileptic drugs (EIAED) within 7 days prior to the first dose of study drug (note: Levetiracetam is not an EIAED and is allowed); (6) other concurrent clinically active malignant disease, requiring treatment, with the exception of non-melanoma cancers of the skin or carcinoma in situ of the cervix or nonmetastatic prostate cancer; (7) nursing or pregnant females; (8) prior exposure to veledimex; (9) use of medications that induce, inhibit, or are substrates of Cytochrome P450 3A4 (CYP3A4) (EC 1.14.13.97) within 7 days prior to veledimex dosing without consultation with the Medical Monitor; (10) presence of any contraindication for a neurosurgical procedure: (11) unstable or clinically significant concurrent medical condition that would jeopardize the safety of a subject and/or their compliance with study protocol (examples may include, but are not limited to, colitis, pneumonitis, unstable angina, congestive heart failure, myocardial infarction within 2 months of screening, and ongoing maintenance therapy for life-threatening ventricular arrhythmia or uncontrolled asthma); and, (12) history of myocarditis or congestive heart failure (as defined by New York Heart Association Functional Classification III or IV), as well as unstable angina, serious uncontrolled cardiac arrhythmia, uncontrolled infection, or myocardial infarction 6 months prior to study entry.

Study Design. Example study includes intratumoral injection of Ad-RTS-hIL12 (2×10¹¹ viral particles [vp]) and 2 escalating doses of veledimex (10 and 20 mg) administered PO in combination with PD-1-specific antibody (e.g., nivolumab) administered intravenously (IV) in subjects with recurrent or progressive glioblastoma. To determine the safe and tolerable dose of Ad-RTS-hIL-12 plus veledimex with nivolumab when administered in combination based on the safety profile observed in the presence of variable corticosteroid use or exposure (such as including: no immediately prior use or exposure (i.e., such that no exogenously administered corticosteroids detectably remain in a subject's system; using then present routine methods of monitoring or detecting); use or exposure to therapeutically low corticosteroid dose(s); use or exposure to therapeutically high corticosteroid dose(s), immediately prior to and/or during the study protocol and treatment).

Subjects may be enrolled into three cohorts to receive two different dose levels of veledimex (e.g., 10 mg or 20 mg) in combination with an PD-1-specific antibody (e.g., nivolumab) at 1 mg/kg or 3 mg/kg. The dose of Ad-RTS-hIL12 may be kept constant (at 2×10¹¹ vp) across cohorts.

For example, in all cohorts subjects may receive anti-PD-1 antibody (e.g., nivolumab) on Day −7. On Day 0, subjects may take one dose of veledimex 3(2) hours prior to injection of Ad-RTS-hIL12. Ad-RTS-hIL12 (2×10¹¹ vp) will be administered by injection on Day 0. The day of Ad-RTS-hIL12 administration is designated as Day 0. Ad-RTS-hIL-12 may be delivered intratumorally or at the margin of the tumor; for example, delivering a total volume of 0.1 mL.

After the Ad-RTS-hIL-12 injection, veledimex may be administered orally; for example, daily for 14 days. The first post craniotomy veledimex dose may be given on Day 1, preferably with food. Subsequent veledimex doses may be taken once daily; for example, in the morning and within approximately 30 minutes of a regular meal. Dosing on Days 2-14 may be at approximately the same time of day (±1 hours) as the Day 1 dosing.

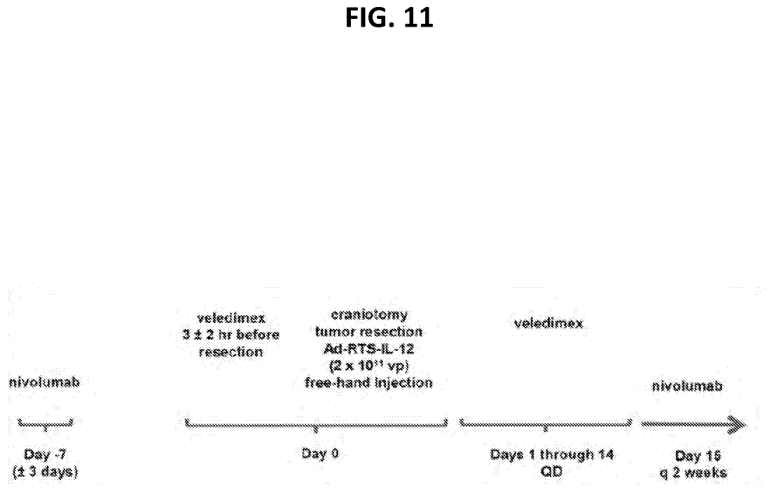

Subjects may receive 1 dose of PD-1-specific antibody (e.g., nivolumab) (for example, either 1 mg/kg or 3 mg/kg) on Day 15 and every two weeks thereafter. Delays in nivolumab dosing may allow for improved therapeutic effect. An example study schema is shown in FIG. 11.

Dose Escalation. Subject dose escalation may proceed according to a standard 3+3 (3 plus 3) study format. For example, a subject in the first cohort may be monitored through Day 28 before the next subject is dosed. In subsequent cohorts, the first subject may be monitored through Day 28 prior to enrolling the second and third subjects in the same cohort. The dose-limiting toxicity (DLT) evaluation period may be defined as Day 0 to Day 28. If a subject receives PD-1-specific antibody (e.g., nivolumab), but not Ad-RTS-hIL-12 plus veledimex, the subject may be replaced to enable assessment of at least 3 subjects for DLTs. Determination of safety and recommendation to dose escalate may occur after all dosed subjects in a cohort have been evaluated for at least 28 days after Ad-RTS-hIL-12 injection. Subjects may receive the cohort-specific dose of PD-1-specific antibody (e.g., nivolumab) on Day 15 and Day 28. After review by a qualified investigator, subjects who received anti-PD-1 antibody (e.g., nivolumab) 1 mg/kg may be permitted to escalate to nivolumab 3 mg/kg for subsequent doses.

Veledimex Dose De-Escalation. If it is determined that dose escalation should not proceed, then dose de-escalation may be undertaken. De-escalation of veledimex dose may be as follows: De-escalation by increments of 5 mg from cohorts in which 2 or more DLTs were observed (e.g., 15 mg down from 20 mg). If in the de-escalation cohort there are fewer than 2 DLTs, the maximum tolerated dose (MTD) may be considered to have been reached or it may be considered to escalate dose by 5 mg (e.g., 15 mg up from 10 mg). In the event of toxicities considered related to PD-1-specific antibody (e.g., nivolumab), individualized management of PD-1-specific antibody (e.g., nivolumab) dosing may be done in accordance with the product label.

Study Duration. The duration of study from the time of initiating subject screening until the completion of survival follow-up may be approximately 42 months, including 18 months for enrollment and 24 months of follow-up. A primary analysis may be performed after the last subject to complete the study reaches 6 months on study. The start of study is defined as the date when the first subject is consented into the study and the study stop date is the date of the last subject's last visit.

Definition of DLT. A DLT is defined as an event occurring in subjects who received nivolumab and Ad-RTS-hIL-12+veledimex from Day 0 to Day 28 that meets any of the following conditions: (1) Any local reaction that requires operative intervention and felt to be attributable to Ad RTS hTL 12+veledimex and nivolumab; (2) Any local reaction that has life threatening consequences requiring urgent intervention or results in death and felt to be attributable to Ad RTS hTL 12+veledimex and nivolumab; (3) Any Grade 3 or greater non-hematological adverse event that is at least possibly related to the Ad RTS hTL 12+veledimex and nivolumab; (4) Any Grade 4 hematologic toxicity that is at least possibly related to Ad RTS hTL 12+veledimex and nivolumab and lasts at least 5 days; (5) Grade 3 or higher thrombocytopenia at least possibly related to Ad RTS hTL 12+veledimex and nivolumab. Diagnostic brain tumor biopsy is not considered a DLT. Fatigue, seizures, headaches, and cerebral edema are commonly observed in this population and will be recorded according to grade of toxicity, but will not be considered a DLT unless a relationship to the combination of Ad-RTS-hIL-12+veledimex and nivolumab is deemed to be the main contributory factor.

Stopping Rules. If any subject, in the DLT evaluation period, experiences a local reaction that requires operative intervention; a local reaction that has life-threatening consequences requiring urgent intervention or results in death; or a grade 4 hematologic toxicity that persists for 5 days, enrollment of new subjects will be paused pending review of the event by the Safety Review Committee. The SRC will make a decision to the enrollment of additional patients at the relevant dose level, to de-escalate veledimex dosing at the relevant dose level, or to amend the protocol prior to enrollment of additional subjects or to discontinue enrollment in the study. In the event that a decision is made to de-escalate dosing, the SRC will evaluate the appropriateness of dosing at a previously evaluated lower dose or exploring an intermediate dose level. If any subject, in the DLT evaluation period, experiences a local reaction that requires operative intervention or a local reaction that has life-threatening consequences requiring urgent intervention or results in death the qualified investigator will discuss the relationship to study drug and determine whether or not to convene an urgent SRC meeting to make a decision to continue active dosing in ongoing subjects.

Definition of MTD. The MTD is defined as the dose level below the dose in which 33% or more subjects of the same cohort experience DLTs. If 2 DLTs occur in the same cohort the dose escalation will stop in the cohort experiencing the DLTs.

Safety Evaluation. Safety will be evaluated in the Overall Safety Population (OSP) and the Evaluable Safety Population (ESP) using National Cancer Institute (NCI) Common Terminology Criteria for Adverse Events (CTCAE) v4.03. In the DLT evaluation period (Day 0 to Day 28) if any subject experiences a local reaction that requires operative intervention and is felt to be attributable to the combination of Ad RTS hTL 12+veledimex and nivolumab; any local reaction that has life-threatening consequences requiring urgent intervention or results in death and is felt to be attributable to the combination of Ad RTS hTL 12+veledimex and nivolumab; or any Grade 4 hematologic toxicity that is at least possibly related to the combination of Ad RTS hTL 12+veledimex and nivolumab and lasts at least 5 days, enrollment of new subjects will be paused pending review of the event by the SRC. Safety assessments will be based on medical review of AE reports and the results of vital signs, physical and neurologic examinations, electrocardiograms (ECGs), clinical laboratory tests, and monitoring the frequency and severity of AEs. The incidence of AEs will be tabulated and reviewed for potential significance and clinical importance. The reporting period of safety data will be from the date of ICF signature through 30 days after the last dose of any study drug.

Evaluation of MTD. Expansion cohorts are not prospectively planned in this substudy. A decision to enroll additional subjects, as part of an expansion cohort, at the determined MTD will be made by the SRC only after the MTD has been identified and safety evaluated, as described in the protocol.

Evaluation of Efficacy. (1) Tumor Response Assessments. The ESP will be evaluated for the Investigator's assessment of ORR, PFS, PSP, and OS. Response will be assessed using iRANO criteria. (2) Immune Response Assessments. Immunological and biological markers, such as, but not limited to, levels of IL 12, interferon gamma (IFN 7), interferon gamma induced protein 10 (IP 10), IL 2, IL 6, IL 10, and neutralizing antibodies to viral components or hIL 12 will be assessed in pretreatment and posttreatment serum samples. (3) Immune cell population markers, such as, but not limited to, cluster of differentiation (CD) antigens CD3, CD4, CD8, CD25, and FOXP3, CD56, CD45RO, natural killer (NK), PD-L1, cytotoxic T lymphocyte associated antigen 4 (CTLA 4), and human leukocyte antigen allele status will be assessed in peripheral blood and tumor. (4) Pharmacokinetics. Veledimex PK will be evaluated at each dose level in the dose escalation and any proposed expansion cohorts.

### Example 6—Expansion Substudy Clinical Protocol for Evaluation of Ad-RTS-hIL-12+Veledimex in Subjects with Recurrent or Progressive Glioblastoma

The following is an example of the parameters which may be used in a (human) clinical protocol to practice the invention: i.e., in the form of the administration of Ad-RTS-IL-12 plus veledimex for the treatment of recurrent glioblastoma or progressive glioblastoma.

Study Objectives. (1) Determine the safety and tolerability of intratumoral Adenovirus RheoSwitch Therapeutic System® (RTS®) human interleukin-12 (Ad-RTS-hIL-12) and oral (PO) veledimex (RTS activator ligand) in subjects with recurrent or progressive glioblastoma. (2) Determine the overall survival (OS) of Ad-RTS-hIL-12+veledimex. (3) Determine the veledimex pharmacokinetic (PK) profile. (4) Determine the veledimex concentration ratio between the brain tumor and blood. (5) Determine (via an investigator's assessment of response) including tumor objective response rate (ORR), progression free survival (PFS), and rate of pseudo-progression (PSP). (6) Evaluate cellular and humoral immune responses elicited by Ad RTS hIL 12+veledimex.

Study Design. Example study of an intratumoral injection of Ad-RTS-hIL-12 (2×10¹¹ viral particles [vp]) and 20 mg of veledimex administered PO in subjects with recurrent or progressive glioblastoma. This study includes a Screening Period, Treatment Period, and Survival Follow-up. After the informed consent form (ICF) is signed, subjects will enter the Screening Period to assess eligibility. The dose of Ad-RTS-hIL-12 2×10¹¹ vp and veledimex 20 mg are constant. The day of Ad-RTS-hIL-12 administration is designated as Day 0. On Day 0 subjects will take one dose of veledimex 3±2 hours prior to injection of Ad-RTS-hIL-12 and Ad-RTS-hIL-12 (2×10¹¹ vp) will be administered by freehand injection. Ad-RTS-hIL-12 will be delivered intratumorally or at the margin of the tumor for a total volume of 0.1 mL. The total amount delivered to each site will be recorded in the CRF. In the event that less than the planned total injected volume is administered, the reason will be provided. Care should be taken to avoid intraventricular or basal cisternal injection or other critical locations. After the Ad-RTS-hIL-12 injection, veledimex will be administered orally for 14 days. The first post craniotomy veledimex dose is to be given on Day 1, preferably with food. Subsequent veledimex doses are to be taken once daily, in the morning and within approximately 30 minutes of a regular meal. Dosing on Days 2-14 should be at approximately the same time of day (+/−1 hours) as the Day 1 dosing.

Eligible Population. An Example study population may include adult subjects with recurrent or progressive Grade IV glioblastoma (herein after referred to as glioblastoma) for which there is no alternative curative therapy. Subjects with Grade III malignant glioma are not eligible to participate in this substudy. Example study population may include Subjects with glioblastoma who are eligible for enrollment who have not previously been treated with bevacizumab for their disease (short use (<4 doses) of bevacizumab for controlling edema is allowed) and who have not received corticosteroids in the previous 4 weeks.

Subject inclusion criteria may include any one or more of: (1) Male or female subject ≥18 and ≤75 years of age; (2) Provision of written informed consent for tumor resection, tumor biopsy, samples collection, and treatment with investigational products prior to undergoing any study-specific procedures; (3) Histologically confirmed supratentorial glioblastoma; (4) Evidence of tumor recurrence/progression by magnetic resonance imaging (MRI) according to Response Assessment in Neuro Oncology (RANO) criteria after standard initial therapy; (5) Previous standard of care antitumor treatment including surgery and/or biopsy and chemoradiation. At the time of registration, subjects must have recovered from the toxic effects of previous treatments as determined by the treating physician. The washout periods from prior therapies are intended as follows: (a) Nitrosureas: 6 weeks; (b) Other cytotoxic agents: 4 weeks; (c) Antiangiogenic agents: 4 weeks (short use (<4 doses) of bevacizumab for controlling edema is allowed); (d) Targeted agents, including small molecule tyrosine kinase inhibitors: 2 weeks; (e) Vaccine-based therapy: 3 months; (6) Able to undergo standard MRI scans with contrast agent before enrollment and after treatment; (7) Karnofsky Performance Status ≥70; (8) Adequate bone marrow reserves and liver and kidney function, as assessed by the following laboratory requirements: (a) Hemoglobin ≥29 g/L; (b) Lymphocytes >500/mm3; (c) Absolute neutrophil count ≥1500/mm3; (d) Platelets ≥100,000/mm3; (e) Serum creatinine ≤1.5× upper limit of normal (ULN); (f) Aspartate transaminase (AST) and alanine transaminase (ALT)≤2.5×ULN. For subjects with documented liver metastases, ALT and AST≤5×ULN; (g) Total bilirubin <1.5×ULN; (h) International normalized ratio (INR) and activated partial thromboplastin time (aPTT) or partial thromboplastin time (PTT) within normal institutional limits. (9) Male and female subjects must agree to use a highly reliable method of birth control (expected failure rate <5% per year) from the Screening Visit through 28 days after the last dose of study drug. Women of childbearing potential (perimenopausal women must be amenorrheic for at least 12 months to be considered of non-childbearing potential) must have a negative pregnancy test at screening.

Subject exclusion criteria may include any one or more of: (1) Radiotherapy treatment within 4 weeks of starting veledimex; (2) Subjects with clinically significant increased intracranial pressure (eg, impending herniation or requirement for immediate palliative treatment) or uncontrolled seizures; (3) Known immunosuppressive disease, or autoimmune conditions, and/or chronic viral infections (eg, human immunodeficiency virus [HIV], hepatitis); (4) Use of systemic antibacterial, antifungal, or antiviral medications for the treatment of acute clinically significant infection within 2 weeks of first veledimex dose. Concomitant therapy for chronic infections is not allowed. Subjects must be afebrile prior to Ad-RTS-hIL-12 injection; only prophylactic antibiotic use is allowed perioperatively; (5) Use of enzyme-inducing antiepileptic drugs (EIAED) within 7 days prior to the first dose of study drug. Note: Levetiracetam (Keppra®) is not an EIAED and is allowed; (6) Other concurrent clinically active malignant disease, requiring treatment, with the exception of non-melanoma cancers of the skin or carcinoma in situ of the cervix or nonmetastatic prostate cancer; (7) Nursing or pregnant females; (8) Prior exposure to veledimex; (9) Use of medications that induce, inhibit, or are substrates of CYP4503A4 within 7 days prior to veledimex dosing without consultation with the Medical Monitor; (10) Presence of any contraindication for a neurosurgical procedure; (11) Unstable or clinically significant concurrent medical condition that would, in the opinion of the Investigator or Medical Monitor, jeopardize the safety of a subject and/or their compliance with the protocol. Examples may include, but are not limited to, colitis, pneumonitis, unstable angina, congestive heart failure, myocardial infarction within 2 months of screening, and ongoing maintenance therapy for life-threatening ventricular arrhythmia or uncontrolled asthma; (12) Previous treatment with bevacizumab for their disease (short use (<4 doses) of bevacizumab for controlling edema is allowed); (13) Subjects receiving systemic corticosteroids during the previous 4 weeks.

Study Duration. The duration of this study from the time of initiating subject screening until the completion of survival follow up is anticipated to be approximately 36 months, including 12 months for enrollment and 24 months of follow-up. The primary analysis will be performed after the last subject to complete the study reaches 12 months on study. The start of study is defined as the date when the first subject is consented into the study and the study stop date is the date of the last subject's last visit.

Stopping Rules. If any subject, in the treatment and Initial Follow-up Period, experiences a local reaction that requires operative intervention; a local reaction that has life-threatening consequences requiring urgent intervention or results in death; a grade 4 hematologic toxicity that persists for 5 days; or death (other than death related to progressive disease) that occurs within 30 days of dosing, enrollment of new subjects will be paused pending review of the event by the Safety Review Committee (SRC). The SRC will make a decision to the enrollment of additional patients at the relevant dose level, to de-escalate veledimex dosing, or to amend the substudy protocol prior to enrollment of additional subjects or to discontinue enrollment in the study. In the event that a decision is made to de-escalate dosing, the SRC will evaluate the appropriateness of dosing at a previously evaluated lower dose or exploring an intermediate dose level. If any subject, in the treatment and Initial Follow-up Period, experiences a local reaction that requires operative intervention or a local reaction that has life-threatening consequences requiring urgent intervention or results in death the qualified investigator will discuss the relationship to study drug and determine whether or not to convene an urgent SRC meeting to make a decision to continue active dosing in ongoing subjects.

Safety Evaluation. Safety will be evaluated in the Overall Safety Population (OSP) and the Evaluable Safety Population (ESP) using National Cancer Institute (NCI) Common Terminology Criteria for Adverse Events (CTCAE) v4.03. Safety assessments will be based on medical review of AE reports and the results of vital signs, physical and neurologic examinations, electrocardiograms (ECGs), clinical laboratory tests, and monitoring the frequency and severity of AEs. The incidence of AEs will be tabulated and reviewed for potential significance and clinical importance. The reporting period of safety data will be from the date of ICF signature through 30 days after the last dose of any study drug.

Evaluation for Efficacy. (1) Tumor Response Assessments: The ESP will be evaluated for the Investigator's assessment of ORR, PFS, PSP, and OS. Response will be assessed using iRANO criteria. (2) Immune Response Assessments: Immunological and biological markers, such as, but not limited to, levels of IL 12, interferon gamma (IFN 7), interferon gamma induced protein 10 (IP 10), IL 2, IL 6, IL 10, and neutralizing antibodies to viral components or hIL 12 will be assessed in pretreatment and posttreatment serum samples. Immune cell population markers, such as, but not limited to, cluster of differentiation (CD) antigens CD3, CD4, CD8, CD25, and FOXP3, CD56, CD45RO, PD-1, PD-L1, and cytotoxic T lymphocyte associated antigen 4 (CTLA 4) will be assessed in peripheral blood and tumor. (3) Pharmacokinetics: Veledimex PK parameters will be evaluated and determined based on plasma levels of veledimex using standard methods and will include, but are not limited to, the maximum plasma concentration (Cmax), time to maximum plasma concentration (Tmax), half life (t½), area under the curve (AUC), volume of distribution (Vd), and clearance (CL).

### Example 7—Clinical Protocol for Evaluation of Ad-RTS-hIL-12+Veledimex in Pediatric Brain Tumor Subjects

The following is an example of parameters which may be used in a (human) clinical protocol to practice the invention: i.e. in the form of the administration of Ad-RTS-hIL-12 plus veledimex in comination with PD-1 specific antibodies for the treatment of pediatric brain tumor subjects, such as Diffuse intrinsic pontine glioma (DIPG) patients.

Target objectives are: (1) Determine the safety and tolerability of intratumoral Ad-RTS-hIL-12 and varying PO veledimex doses in pediatric brain tumor subjects; (2) Determine the recommended Phase II veledimex dose in pediatric brain tumor subjects when given with intratumoral Ad-RTS-hIL-12; (3) Determine the pharmacokinetics (PK) of veledimex in subjects treated with Ad-RTS-hIL-12+veledimex; (4) Determine the veledimex concentration ratio between the brain tumor and blood in subjects treated with Ad-RTS-hIL-12+veledimex (Arm 1 only); (5) Evaluate cellular and humoral immune responses elicited by Ad-RTS-hIL-12+veledimex in pediatric brain tumor subjects; (6) Determine investigator assessment of response, including tumor objective response rate (ORR) and progression-free survival (PFS) of subjects treated with Ad-RTS-hIL-12+veledimex; (7) Determine overall survival (OS) of subjects treated with Ad-RTS-hIL-12+veledimex; (8) Assess the value of tumor and/or blood markers in predicting response to treatment.

Study Design. Example study includes Ad-RTS-hIL-12 administered by intratumoral injection and varying PO veledimex doses in pediatric brain tumor subjects. This study will investigate one fixed intratumoral Ad-RTS-hIL-12 dose (2×10¹¹ viral particles [vp]) and escalating veledimex doses to determine the safe and tolerable Phase II pediatric dose based on the safety profiles observed in the presence of variable corticosteroid exposure. Example study is divided into 3 periods: the Screening Period, the Treatment Period, and the Follow-up Period (Initial and Long Term). After the informed consent form (ICF) or subject assent, as applicable, is signed, subjects will enter the Screening Period to assess eligibility. Eligible subjects will be stratified into one of 2 arms, according to diagnosis. Arm 1 is open to pediatric brain tumor subjects who are scheduled for a standard-of-care craniotomy and tumor resection, with the exclusion of subjects with diffuse intrinsic pontine glioma (DIPG). Arm 2 is open only to subjects with DIPG who are post prior standard focal radiotherapy (≥2 weeks and ≤10 weeks). Arm 1 subjects will receive one veledimex dose before the resection procedure. Samples (tumor, blood, and cerebrospinal fluid [CSF] [if available]) will be collected as described below. After Ad-RTS-hIL-12 intratumoral injection, Arm 1 subjects will continue on PO veledimex for 14 days for a total of 15 doses of veledimex. Arm 2 subjects will receive a single Ad RTS hIL-12 (2×10¹¹ vp) dose by stereotactic injection and will receive PO veledimex for 14 days.

Arm 1: Pediatric brain tumor subjects scheduled for craniotomy and tumor resection (excluding DIPG). Subjects with a clinical indication for tumor resection will receive veledimex 3 (±2) hours before the craniotomy procedure, on an empty stomach (excluding other medications). At the time of tumor resection, brain tumor, blood, and CSF (if available) samples will be collected to determine the veledimex concentration ratio between brain tumor, blood, and CSF (if available). Immediately after tumor resection, Ad-RTS-hIL-12 (2×10¹¹ vp) will be administered by freehand injection into approximately 2 sites within the residual tumor for a total volume of 0.1 mL. The total amount delivered to each site will be recorded in the case report form (CRF). In the event that less than the planned total injected volume is administered, the reason will be provided. Care should be taken to avoid intraventricular or basal cisternal injection or other critical locations. The day of Ad-RTS-hIL-12 administration is designated as Day 0. When available, an intra-operative magnetic resonance imaging (MRI) scan should be performed to guide the Ad-RTS-hIL-12 injection to areas of contrast-enhancing tumor tissue. After the Ad-RTS-hIL-12 injection, PO veledimex will be administered once daily (QD) for 14 days. The first postresection veledimex dose is to be given on Day 1, preferably in the morning and within approximately 30 minutes of completion of a regular meal. There should be a minimum of 10 hours between veledimex doses. Subsequent veledimex doses (Days 2 to 14) are to be taken at approximately the same time of day (±1 hour) as the Day 1 dosing and within approximately 30 minutes of completion of a regular meal.

Arm 2: Subjects with DIPG who will not undergo tumor resection. Subjects with DIPG who will not undergo tumor resection will receive Ad-RTS-hIL-12 by standard stereotactic surgery on Day 0. At the time of stereotactic surgery, brain tumor biopsy and blood samples will be collected. Ad-RTS-hIL-12 (2×10¹¹ vp) will be administered by stereotactic injection into the intratumoral site. The day of Ad-RTS-hIL-12 administration is designated as Day 0. Ad-RTS-hIL-12 will be delivered into the intratumoral site or into the periphery of the tumor for a total volume of 0.1 mL. The total amount delivered to each site will be recorded in the CRF. In the event that less than the planned total injected volume is administered, the reason will be provided. Care should be taken to avoid intraventricular or basal cisternal injection or other critical locations. After the Ad-RTS-hIL-12 injection, PO veledimex will be administered QD for 14 days. The first veledimex dose is to be given on Day 1, preferably in the morning and within approximately 30 minutes of completion of a regular meal. Subsequent veledimex doses (Days 2 to 14) are to be taken QD and at approximately the same time of day (±1 hour) as the Day 1 dosing and within approximately 30 minutes of completion of a regular meal. There should be a minimum of 10 hours between veledimex doses.

Cohorts: For example, 2 veledimex doses are used (10 mg and 20 mg). Subject enrollment and veledimex dose escalation will proceed according to a standard 3+3 design, modified to independently evaluate 2 groups (ie, arms) of subjects that may exhibit different safety and tolerability profiles, with the first cohort of each arm receiving 10 mg veledimex followed by the second cohort of each arm receiving 20 mg veledimex. The study arms and assigned doses may be divided into 4 cohorts. Cohort 1—Arm 1, Craniotomy Procedure, 10 mg veledimex dose (BSA-adjusted dose); Cohort 2—Arm 1, Craniotomy Procedure, 20 mg veledimex dose (BSA-adjusted dose); Cohort 3—Arm 2, Streotactic Procedure, 10 mg veledimex dose (BSA-adjusted dose); Cohort 4—Arm 2, Streotactic Procedure, 20 mg veledimex dose (BSA-adjusted dose).

Each cohort will consist of subjects ≤21 years-of-age who meet eligibility criteria. Once the last subject in Cohort 1 completes the dose-limiting toxicity (DLT) evaluation period and the SRC has approved, enrollment may be opened for Cohort 2 and Cohort 3. Once the last subject in Cohort 3 completes the DLT evaluation period and the SRC has approved, enrollment may be opened for Cohort 4. Each subject in each cohort will be monitored for 28 days after Ad-RTS-hIL-12 injection before additional subjects are enrolled in the same cohort. The evaluation period for DLT is 28 days after Ad RTS-hIL-12 injection (Day 0 to Day 28). Determination of safety and the recommendation to dose escalate will occur after all dosed subjects in a cohort have been evaluated for at least 28 days after Ad-RTS-hIL-12 injection.

Study Population. Example study population includes pediatric subjects with a) recurrent or refractory supratentorial brain tumors, not in direct continuity with the ventricular system, that are unresponsive to conventional treatment or for which there is no alternative curative therapy and b) DIPG post prior standard focal radiotherapy and for which a biopsy has previously been obtained

Criteria for a target subject population may include: (1) Male or female subjects ≤21 years-of-age with the demonstrated ability to swallow capsules whole and who are willing to provide access to previously obtained biopsy results; (2) Provision of written informed consent and assent, when applicable, for tumor resection, stereotactic surgery, tumor biopsy, sample collection, and/or treatment with study drug prior to undergoing any study-specific procedures; (3) Arm 1: Evidence of recurrent or progressive supratentorial tumor, which has shown a >25% increase in bi dimensional measurements by MRI or is refractory with significant neuro deterioration that is not otherwise explained with no known curative therapy, not in direct continuity with the ventricular system (e.g., there is physical separation between the tumor and ventricule, the tumor does not open directly into the ventricular system). Arm 2: Clinical presentation of DIPG and compatible MRI with approximately ⅔ of the pons included. Subject should be ≥2 weeks and ≤10 weeks post standard focal radiotherapy (ie, dose of 5400 to 5960 cGy and maximum dexamethasone of 1 mg/m2/day); (4) At the time of registration, subjects must have recovered from the toxic effects of previous treatments, as determined by the treating physician. The washout periods from prior therapies are intended as follows: (a) Targeted agents, including small-molecular tyrosine kinase inhibitors: 2 weeks; (b) Other cytotoxic agents: 3 weeks; (c)Nitrosoureas: 6 weeks; (d) Monoclonal antibody immunotherapies (eg, PD-1, CTLA-4): 6 weeks; (e) Vaccine-based and/or viral therapy: 3 months; (5) On a stable or decreasing dose of dexamethasone for the previous 7 days; (6) Able to undergo standard MRI scans with contrast agent before enrollment and after treatment; (7) Have age-appropriate functional performance: (a) Lansky score ≥50 or; (b) Karnofsky score >50 or; (c) Eastern Cooperative Oncology Group (ECOG) score ≤2; (8) Have adequate bone marrow reserves and liver and kidney function, as assessed by the following laboratory requirements: (a) Hemoglobin ≥8 g/L; (b) Absolute lymphocyte count ≥500/mm3; (c) Absolute neutrophil count ≥1000/mm3; (d) Platelets ≥100,000/mm3 (untransfused [>5 days] without growth factors); (e) Serum creatinine ≤1.5× upper limit of normal (ULN) for age; (f) Aspartate transaminase (AST) and alanine transaminase (ALT) ≤2.5×ULN for age; (g) Total bilirubin <1.5×ULN for age; (h) International normalized ratio (INR) and activated thromboplastin time within normal institutional limits; (9) Male and female subjects of childbearing potential must agree to use a highly reliable method of birth control (expected failure rate <1% per year) from the Screening Visit through 28 days after the last dose of study drug. Women of childbearing potential must have a negative pregnancy test at screening.

Subject exclusion criteria may include any one or more of: (1) Radiotherapy treatment prior to the first veledimex dose: (a) Focal radiation ≤4 weeks; (b) Whole-brain radiation ≤6 weeks; (c) Cranio-spinal radiation ≤12 weeks; Subjects in Arm 2 (ie, with DIPG) must be ≥2 weeks and ≤10 weeks after standard focal radiotherapy (dose of 5400 to 5960 cGy and maximum dexamethasone of 1 mg/m2/day); (2) Subjects with clinically significant increased intracranial pressure (eg, impending herniation or requirement for immediate palliative treatment) or uncontrolled seizures; (3) Subjects whose body surface area (BSA) would expose them to <75% or >125% of the target dose per the provided dosing table; (4) Known immunosuppressive disease, autoimmune condition, and/or chronic viral infection (eg, human immunodeficiency virus [HIV], hepatitis); (5) Use of systemic antibacterial, antifungal, or antiviral medications for the treatment of acute clinically significant infection within 2 weeks of first veledimex dose. Concomitant therapy for chronic infections is not allowed. Subjects must be afebrile prior to Ad-RTS-hIL-12 injection; only prophylactic antibiotic use is allowed perioperatively; (6) Use of enzyme-inducing antiepileptic drugs (EIAEDs) within 7 days prior to the first dose of study drug. See Appendix 4 for prohibited and permitted antiepileptic drugs; (7) Other concurrent clinically active malignant disease, requiring treatment; (8) Nursing or pregnant females; (9) Prior exposure to veledimex; (10) Use of medications that induce, inhibit, or are substrates of cytochrome p450 (CYP450) 3A4 within 7 days prior to veledimex dosing without consultation with the Medical Monitor; (11) Use of heparin or acetylsalicylic acid (ASA) without consultation with the Medical Monitor; (12) Presence of any contraindication for a neurosurgical procedure; (13) Unstable or clinically significant concurrent medical condition that would, in the opinion of the Investigator as agreed to by the Medical Monitor, jeopardize the safety of a subject and/or their compliance with the protocol

Safety Evaluation. The first level of safety oversight will occur through the site Investigator and Medical Monitor. A formal Safety Review Committee (SRC), comprised of the study Investigators, the Medical Monitor, and other appropriate Sponsor representatives, will provide the overall safety oversight. Additional external medical and scientific experts may also be invited to participate in the reviews, as needed. A separate charter will outline the SRC activities. Briefly, the SRC will evaluate subject safety within each cohort. If no significant safety events occur with the first subject of each cohort, the second and third subjects will be enrolled and treated. If a significant safety event occurs with the first subject, the SRC will convene to evaluate the safety event(s) and to make a recommendation and decision on the enrollment of the second and third subjects in the same cohort. Upon completion of each cohort, the SRC will meet to review the data collected to determine if enrollment in subsequent cohorts may begin. Enrollment in cohorts with the 20 mg assigned veledimex dose (ie, Cohorts 2 and 4) will not commence until the SRC has determined that dosing at the lower level (ie, Cohorts 1 and 3, as applicable) did not result in DLTs that would preclude dose escalation. In addition to recommending dose escalation or the opening of the Arm 2 cohorts, the SRC will determine if an expansion cohort(s) should be allowed. In the event that the SRC determines that escalation and/or expansion is not warranted, a decision will be made about stopping the investigation. At the discretion of the SRC, the investigation may be continued at a lower dose.

Study Drug Dose and Mode of Administration. (1) Ad-RTS-hIL-12 will be administered by either freehand injection into residual tumor sites immediately after tumor resection (Arm 1) or by stereotactic injection into the intratumoral site (Arm 2). (2) Veledimex will be administered PO. There should be a minimum of 10 hours between veledimex doses.

Arm 1: (Cohorts 1 and 2) will receive veledimex 3 (±2) hours before the planned craniotomy, and will continue veledimex dosing after Ad-RTS-hIL-12 administration for an additional 14 days. Subsequent veledimex doses (Days 1 to 14) are to be taken QD and at approximately the same time of day (±1 hour) as the Day 1 dosing and within approximately 30 minutes of completion of a regular meal.

Arm 2: (Cohorts 3 and 4) will receive veledimex only after Ad-RTS-hIL-12 administration for 14 days. The first veledimex dose is to be given on Day 1, preferably in the morning and within approximately 30 minutes of completion of a regular meal. Subsequent veledimex doses (Days 2 to 14) are to be taken QD and at approximately the same time of day (±1 hour) as the Day 1 dosing and within approximately 30 minutes of completion of a regular meal.

Based on the Phase III dose (20 mg [approximately 10.6 mg/m2]) in the adult population, this study will explore the following BSA-adjusted veledimex doses given after Ad-RTS-hIL-12 2×10¹¹ vp) administration. The starting dose in Cohort 1 will be 10 mg, which is approximately 5.3 mg/m2. The actual administered dose will depend on the subject's BSA and available capsule sizes. Because veledimex is an oral agent and is supplied in fixed capsule sizes (5 mg and 20 mg), the actual administered dose is based on a subject's BSA and is bound by the rounding constraints set by 5 mg. The Sponsor developed a BSA-adjusted dosing algorithm designed to enable dosing within 25% of the target mg/m2 dose. If a subject's BSA would expose the subject to <75% or >125% of the target assigned dose, the actual administered dose will be modified to ensure that the target mg/m2 dose is achieved. Minimum BSA restrictions for enrollment must be met in order for a subject to be appropriately dosed. Potential subjects whose BSAs do not have a correlated administered dose may be enrolled at the discretion of the Investigator and the Medical Monitor, but will not be considered in the assessment of the recommended pediatric Phase II dose. Dosing of these subjects can only commence once the cohort has been reviewed by the SRC and determined that the dosing at the specified level (10 mg or 20 mg) is appropriate for escalation or as the recommended Phase 2 pediatric dose. These subjects will be analyzed separately.

Table 9 illustrates this algorithm and captures the BSA-adjusted actual administered dose that subjects would receive at assigned dose levels based on a minimum capsule size of 5 mg.

TABLE 9
BSA-adjusted dosages

| | | | Min | | | Max | | | |
|---|---|---|---|---|---|---|---|---|---|
| | | | BSA | % of | | BSA | % of | | |
| | Target | Min | Target | Expected | Max | Target | Expected | Actual | |
| Cohort | Dose | BSA | Dose | Dose | BSA | Dose | Dose | Dose ^{a} | |
| 10 mg | 5.3 | 0.5 | 10 | 189% | 0.75 | 6.7 | 126% | 5 | mg^{b} |
| 10 mg | 5.3 | 0.76 | 6.6 | 124% | 1.26 | 4.0 | 75% | 5 | mg |
| 10 mg | 5.3 | 1.27 | 3.9 | 74% | 1.5 | 3.3 | 63% | 5 | mg |
| 10 mg | 5.3 | 1.27 | 7.9 | 149% | 1.5 | 6.7 | 126% | 10 | mg^{b} |
| 10 mg | 5.3 | 1.51 | 6.6 | 125% | 2.53 | 4.0 | 75% | 10 | mg |
| | | | Min | | | Max | | | |
| | | | BSA | % of | | BSA | % of | | |
| | Target | Min | target | expected | Max | target | Expected | Actual | |
| Cohort | Dose | BSA | dose | dose | BSA | dose | Dose | Dose ^{a} | |
| 20 mg | 10.6 | 0.5 | 10 | 94% | 0.63 | 7.9 | 75% | 5 | mg |
| 20 mg | 10.6 | 0.64 | 7.8 | 74% | 0.75 | 6.7 | 63% | 5 | mg |
| 20 mg | 10.6 | 0.64 | 15.6 | 147% | 0.75 | 13.3 | 126% | 10 | mg^{b} |
| 20 mg | 10.6 | 0.76 | 13.2 | 124% | 1.25 | 8.0 | 75% | 10 | mg |
| 20 mg | 10.6 | 1.26 | 11.9 | 112% | 1.87 | 8.0 | 75% | 15 | mg |
| 20 mg | 10.6 | 1.88 | 10.6 | 100% | 2.53 | 7.9 | 75% | 20 | mg |

^{a} The actual dose is ± 25% of the target dose.
^{b}Subjects in this BSA range may be dosed at the discretion of the Investigator and the Medical Monitor

Dose Escalation. Each subject in each cohort will be monitored for 28 days before subsequent subjects are enrolled. The SRC will convene after the final subject in Cohort 1 completes the 28-day DLT evaluation period. The SRC will make a recommendation regarding: (1) Opening enrollment of Cohort 2 or discontinuing the investigation; (2) Opening enrollment of Cohort 3; If the SRC recommends enrollment of Cohorts 2 and 3, those cohorts will open in parallel, and each subject in each cohort will be monitored for 28 days before subsequent subjects are enrolled in the same cohort. The SRC will convene once the final subjects in each cohort complete the 28-day DLT evaluation period. Cohorts 2 and 3 will be reviewed independently by the SRC. The SRC will make a recommendation regarding (1) Expansion of Cohort 1 or expansion of Cohort 2; (2) Expansion of Cohort 3 or opening enrollment of Cohort 4

Dose De-Escalation. The SRC will recommend either that the cohort continue at the existing veledimex dose, begin dosing at a lower dose level, or that other measures be undertaken, including discontinuation of treatment. If it is determined that escalation should not proceed, dose de-escalation may be undertaken and the SRC will consider de-escalating the veledimex dose as follows: (1) De-escalation by increments of 5 mg from the cohort in which 2 or more DLTs were observed (eg, 15 mg, de-escalated from 20 mg); (2) If there are 2 or more DLTs in the dose de-escalation cohort, the SRC will consider de-escalating the veledimex dose by an additional increment of 5 mg (eg, 5 mg down from 10 mg) or declaring a previously studied dose level the recommended pediatric Phase II dose.

Definition of DLT. DLT is defined as an event occurring within the first 28 days (ie, Day 0 to Day 28) that meets at least one of the following conditions: (1) Any local reaction that requires operative intervention and is felt to be attributable to study drug; (2) Any local reaction that has life-threatening consequences requiring urgent intervention or results in death and is felt to be attributable to study drug; (3) Any Grade 3 or higher non-hematologic adverse event that is at least possibly related to study drug and lasts 3 3 days; (4) Nausea and vomiting will not be considered a DLT unless at least Grade 3 and refractory to antiemetics; (5) Grade 3 or higher thrombocytopenia (<50,000/mm3) at least possibly related to study drug; (6) Any Grade 4 hematologic toxicity (except thrombocytopenia) that is at least possibly related to study drug and lasts ≥5 days; (7) Dose escalation may be stopped by the Medical Monitor before a DLT is observed, but where the observed toxicities indicate the strong likelihood of unacceptable toxicity at higher doses. Diagnostic brain tumor biopsy is not considered a DLT. Seizures, headache, and cerebral or pontine edema are commonly observed in this population and will be recorded according to the grade of toxicity, but will not be considered a DLT unless a relationship to study drug is deemed to be the main contributory factor. Transient neurological changes are expected in Arm 2 and will not be considered a DLT unless they last >10 days. Expansion cohorts at the recommended pediatric Phase II dose will be allowed in each arm if deemed appropriate by the SRC. In Arm 1, enrollment into an expansion cohort may be limited to a specific tumor type based on data collected in the dose-escalation cohorts. A decision to enroll additional subjects in an expansion cohort at the Ad-RTS-hIL-12 and veledimex dose will be made by the SRC. If an expansion cohort is implemented, the veledimex dose may be delayed or reduced for individual subjects in the event of toxicity. If ≥33% of subjects in the expansion cohort experience DLTs, using the definition in the dose-escalation phase, additional subjects may be enrolled at the next lower dose tested in the dose-escalation phase or at an intermediate dose, as recommended by the SRC.

Definition of Recommended Pediatric Phase II Dose. The recommended pediatric Phase II veledimex dose will be determined from the Evaluable Safety Population (ESP), as defined below. The recommended pediatric Phase II dose is defined as the dose level below the dose in which ≥33% of subjects in the same cohort experience DLTs. If 2 DLTs occur in the same cohort, dose escalation will stop in the cohort experiencing the DLTs.

Safety Evaluation. Safety will be evaluated in the Overall Safety Population (OSP) and the Evaluable Safety Population (ESP), as defined below, using National Cancer Institute (NCI) Common Terminology Criteria for Adverse Events (CTCAE) v4.03. In the DLT evaluation period (Day 0 to Day 28), if any subject experiences a local reaction that requires operative intervention and is felt to be attributable to study drug(s); any local reaction that has life-threatening consequences requiring urgent intervention or results in death and is felt to be attributable to study drug; or any Grade 4 hematologic toxicity, except thrombocytopenia, that is at least possibly related to study drug and lasts ≥5 days, enrollment of new subjects will be paused pending review by the SRC. Safety assessments will be based on medical review of adverse event reports and the results of vital signs, physical and neurologic examinations, electrocardiograms (ECGs), clinical laboratory tests, and monitoring of the frequency and severity of adverse events. The incidence of adverse events will be tabulated and reviewed for potential significance and clinical importance. Urine, fecal, saliva, buccal, and blood samples will be collected and tested for viral replication. The reporting period for safety data will be from the date of ICF or assent signature through the Initial Follow-Up Period.

Criteria for Evaluation. (1) Tumor Response Assessments and Overall Survival: The ESP will be evaluated for Investigator assessment of ORR, PFS, and OS. Response will be assessed using the baseline (Day 2) Immunotherapy Response Assessment for Neuro-Oncology (iRANO) criteria used to characterize tumor response assessments. In the absence of a pediatric RANO criteria, adult response criteria will be used. (2) Immune Response Assessments: Immunologic and biologic markers, such as levels of IL-12, IFN-γ, IFN-γ-induced protein 10 (IP-10), IL-2, IL-6, IL-10, and neutralizing antibodies to viral components or hIL-12 will be assessed in pre- and post treatment serum samples. (3) Immune cell population markers such as cluster of differentiation (CD) antigens CD3, CD4, CD8, CD25, and FOX-P3, CD56, CD45RO, and human leukocyte antigen allele status will be assessed as scheduled in the Schedule of Study Procedures. (4) Pharmacokinetic Evaluations: Veledimex PK parameters will be evaluated at each dose level in the dose escalation and any proposed expansion cohorts for subjects in Arms 1 and 2.

Statistical Methods. (1) Analysis Populations: (a) The OSP includes all subjects who received at least 1 dose of veledimex (pre-tumor resection and/or post-stereotactic procedure) and/or all subjects who received Ad RTS hIL-12; (b) The ESP includes all subjects who received Ad-RTS-hIL-12 and at least 1 dose of veledimex after Ad-RTS-hIL-12 administration; (c) The Pharmacokinetics Population (PKP) includes all subjects who received veledimex with sufficient time points; (2) Safety Analysis: The OSP will be used to perform safety evaluations for all safety variables. The ESP will be used to make decisions regarding escalation to higher veledimex doses for Arms 1 and 2 separately, based on a standard 3+3 design, as previously described. For the first (10 mg) veledimex dose cohorts in Arms 1 and 2 (ie, Cohorts 1 and 3, respectively), a minimum of 3 ESP subjects must be eligible for evaluation of safety. In addition, evaluation of any DLTs will be performed according to protocol-defined criteria. Safety variables will be tabulated and presented by arm and by dose cohort. Exposure to study drug(s) and reasons for discontinuation of study treatment will be tabulated. All treatment-emergent adverse events (TEAEs) will be coded according to the System Organ Class and Preferred Term using the Medical Dictionary for Regulatory Activities (MedDRA). The TEAEs will be tabulated by the number and percent of subjects according to relationship to study drug(s), severity, and seriousness. Laboratory parameters will be summarized by visit. Vital signs and physical examination data will be listed by visit; (3) Tumor Response and Overal Survival Analyses: Tumor response analysis will be performed on the ESP. The Investigator assessment of ORR and PFS will be determined for each cohort. The OS is defined as the duration of time from the first dose of study drug to the date of death or, for subjects who are still alive 2 years after first dose of study drug, subjects will be censored at the last follow-up contact date. A 2-sided confidence interval will be computed for the ORR. The PFS and OS will be analyzed using Kaplan-Meier methods; (4) Pharmacodynamic, PK, and Immunologic Analyses: Veledimex PK parameters will be determined based on blood (plasma) levels of veledimex using WinNonLin Phoenix 64. Available pharmacodynamic, immunologic, and biologic response marker data will be summarized by cohort and by visit.

Sample Size Determination. The choice of the number of subjects was based on the standard 3+3 design, modified for independent evaluation of 2 subject arms that may exhibit different safety and tolerability profiles. Approximately 24 subjects may be enrolled into this study, including 3 to 6 subjects per cohort. Subjects who withdraw from the study during the DLT evaluation period (Day 0 to Day 28) for reasons other than toxicity or disease progression may be replaced.

Study Duration. The duration of this study from the time of initiating subject screening until completion of survival follow-up is anticipated to be approximately 48 months, including 24 months for enrollment and 24 months for follow-up. The study start is defined as the date when the first subject is consented into the study; the study stop date is the date of the last protocol-defined assessment in the Survival Follow-up Period.

### Example 8—Administration of Ad-RTS-hIL-12 and Veledimex as a Monotherapy in Subjects with Recurrent or Progressive Glioblastoma or Malignant Glioma

Background: Ad-RTS-hIL-12 (Ad) is a recombinant, adenoviral-delivered, gene therapy for expression of interleukin-12 (IL-12) under the control of an orally administered activator ligand, veledimex (V), acting in concert with a ligand-inducible gene switch (also referred to as “RTS©”). Administration of Ad-RTS-hIL-12 provides for elicitation of an anti-cancer effector T cell response while concurrently providing ability to control and/or reduce adverse effects which may be caused by IL-12 over-expression and/or an undesirable degree of IL-12 systemic toxicity.

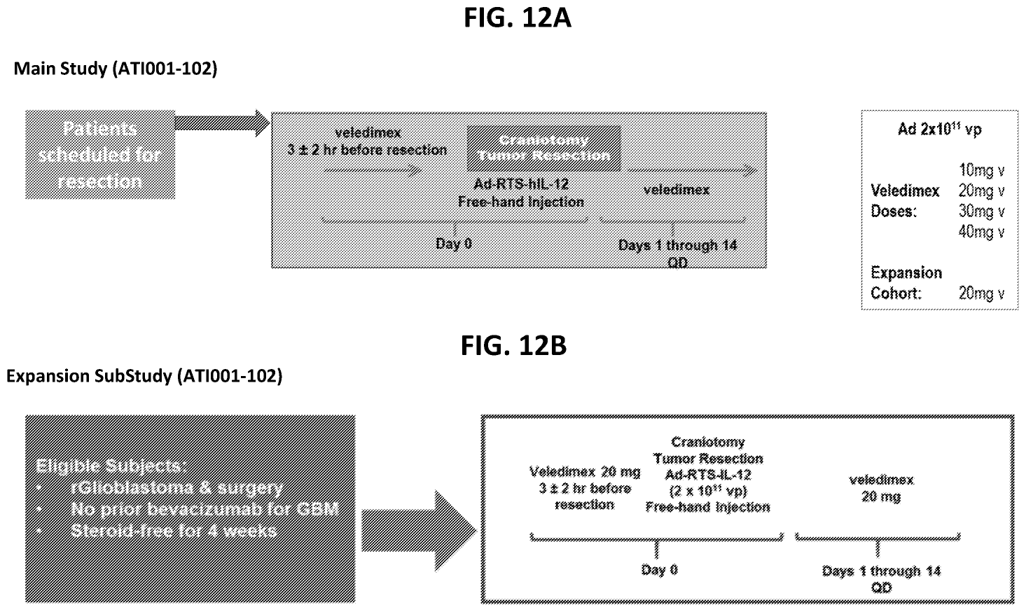

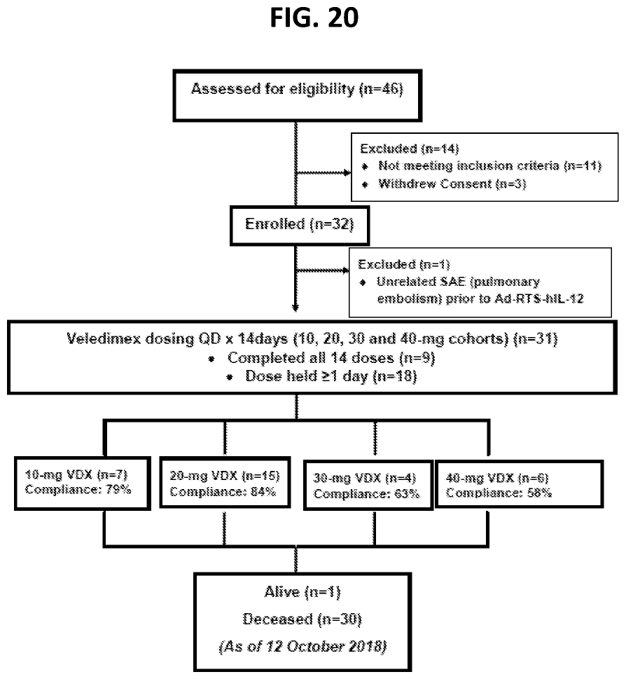

Methods (Main Study): An open label, single arm Phase 1 study evaluating safety and tolerability of local, inducible IL-12 expression in adult subjects with recurrent or progressive glioblastoma (rGBM) or Grade III malignant glioma glioblastoma was commenced; refer to FIG. 12A (“Main Study”) for schema and FIG. 20 for CONSORT flow diagram. This study is investigating two intratumoral Ad-RTS-hIL-12 doses (2×10¹¹ vp or 1×10¹² vp) and escalating veledimex doses (10 mg to 40 mg) to determine the safe and tolerable dose based on the safety profiles observed in the presence of variable corticosteroid exposure. Group 1 received one veledimex dose before a standard-of-care resection procedure, Ad was administered by freehand intratumoral injection, then continued with oral V QD for 14 days. Subjects not scheduled for tumor resection (Group 2) received Ad-RTS-hIL-12 (2×10¹¹ vp or 1×10¹² vp) by stereotactic injection and then continued on oral veledimex for 14 days. An open label, single arm Phase 1 study evaluating safety and tolerability of local, inducible IL-12 expression in adult subjects with recurrent glioblastoma (rGBM) who were bevacizumab naïve (i.e., not previously treated with bevacizumab) and non-steroid dependent during the 4 weeks prior to Ad injection was commenced; see FIG. 12A (“Main Study”). Ad was administered by intratumoral injection (at doses of 2×10¹¹ vp) with oral V (at 20 mg/dose) QDx15 doses.

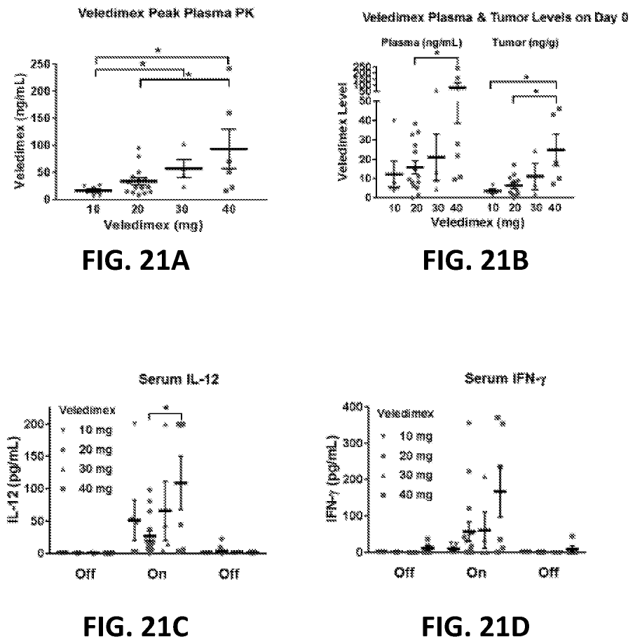

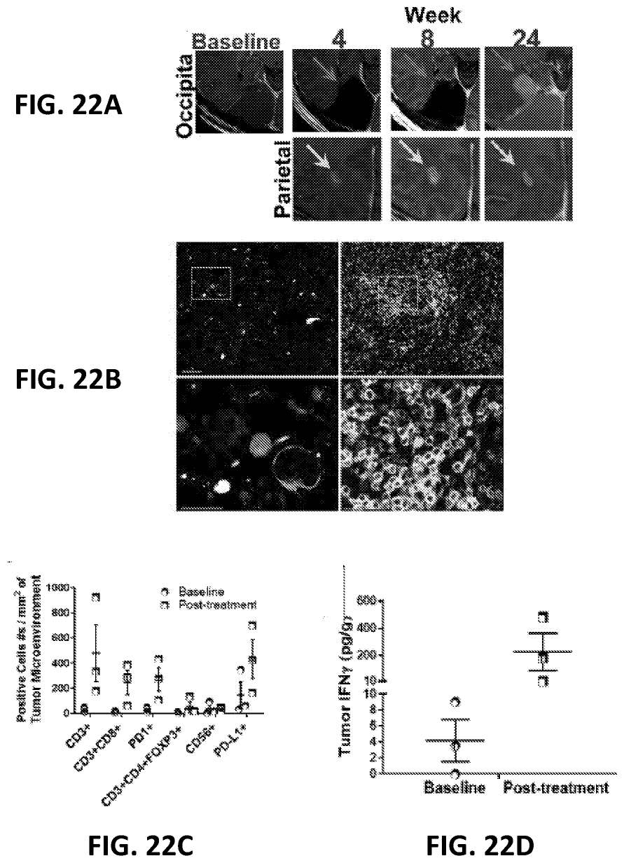

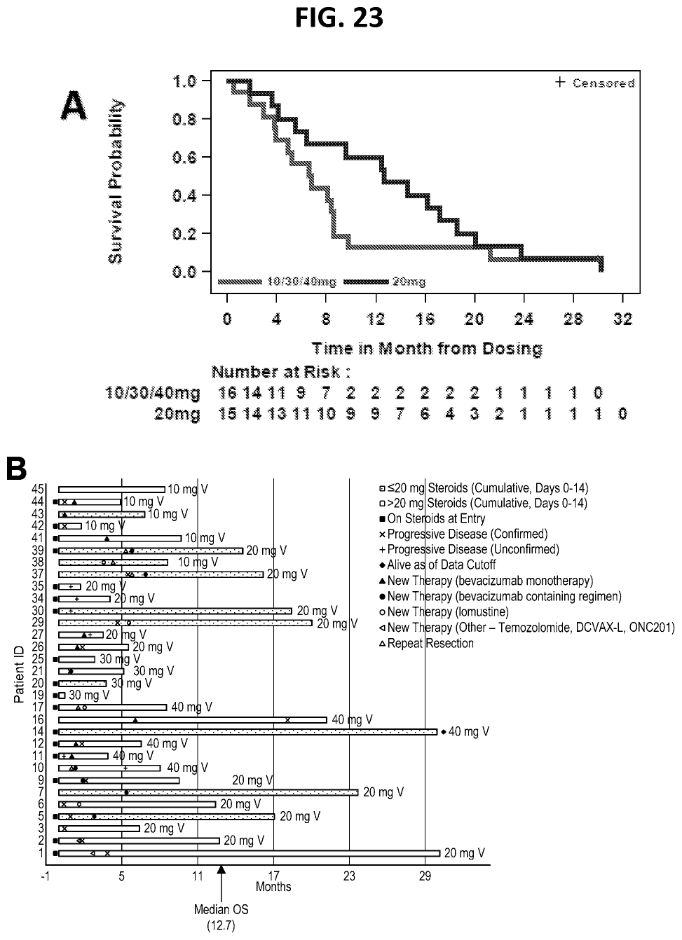

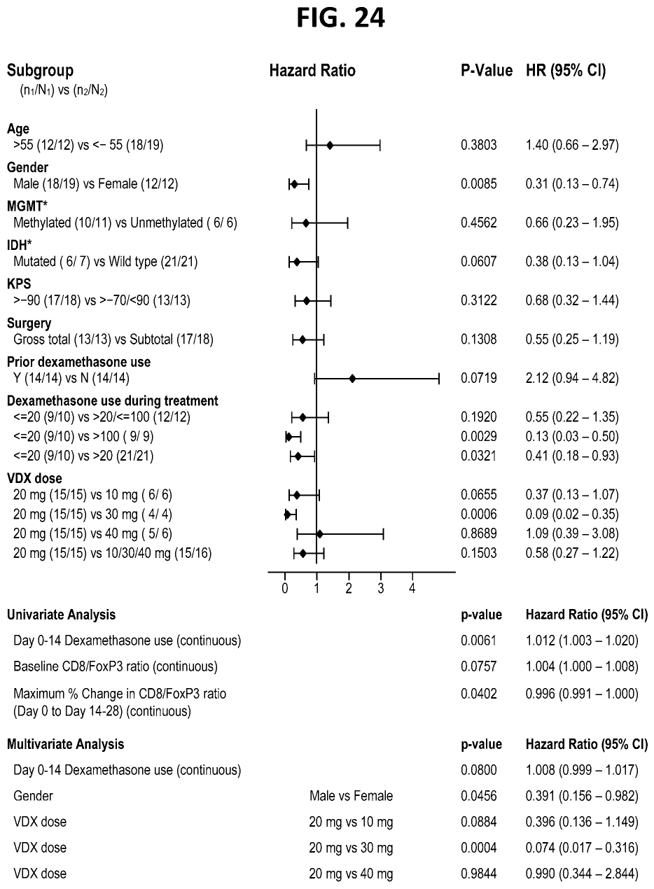

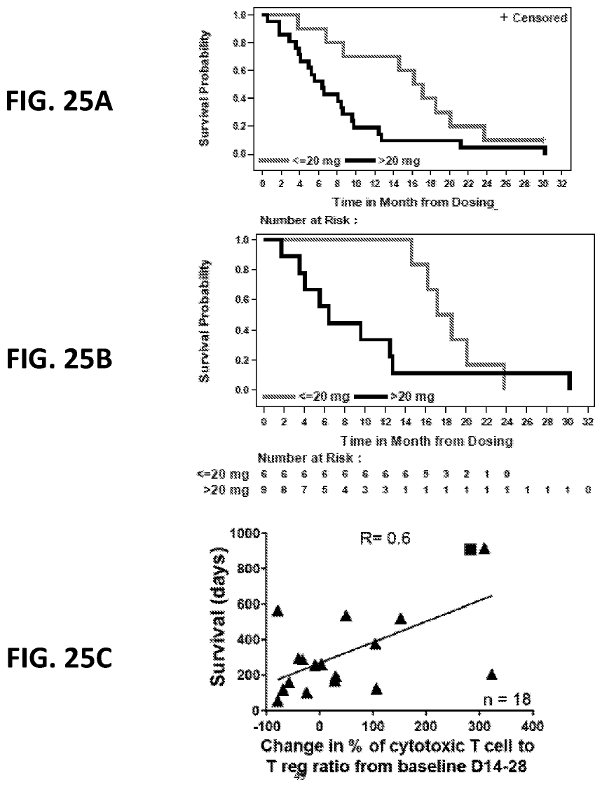

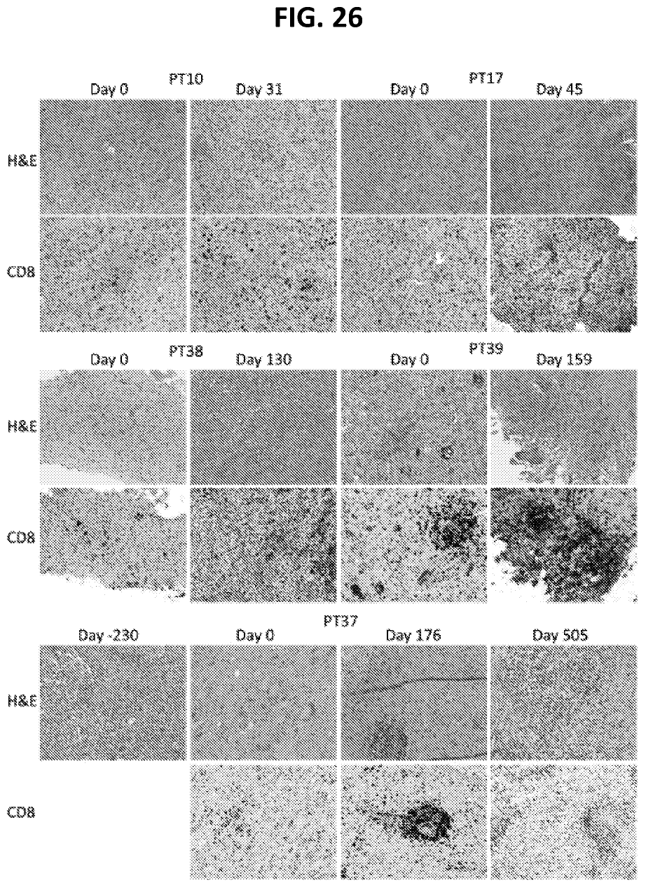

Results (Main Study): In the Main Study, dose-related increases in V, IL-12 and interferon-γ, were observed in peripheral blood with approximately 40% V tumor penetration (FIGS. 21A-21D). Three (±2) hours after V administration, the peak IL-12 serum concentration across the four cohorts was 25-109 pg/mL, while the peak IFN-γ serum concentration was 15-168 pg/mL. Frequency and severity of adverse events, including cytokine release syndrome, correlated with V dose, reversing promptly upon discontinuation. 20 mg V had superior drug compliance and 12.7 months median overall survival (mOS) at mean follow-up of 13.1 months (FIGS. 23A-23B and FIG. 24). Concurrent corticosteroids negatively impacted survival: in patients receiving >20 mg versus ≤20 mg dexamethasone cumulatively (Days 0-14), mOS was 6.4 months versus 16.7 months, respectively, in all patients and 6.4 months and 17.8 months, respectively in the 20 mg V cohorts (FIGS. 25A-25C). Reresection in 5/5 subjects from the Main Study with suspected after Ad+V treatment revealed mostly pseudoprogression with increased CD8⁺ tumor-infiltrating lymphocytes producing IFN-γ and also PD-1 (FIGS. 22A-22D and FIG. 26). These new inflammatory infiltrates support an immunological anti-tumor effect of hIL-12.

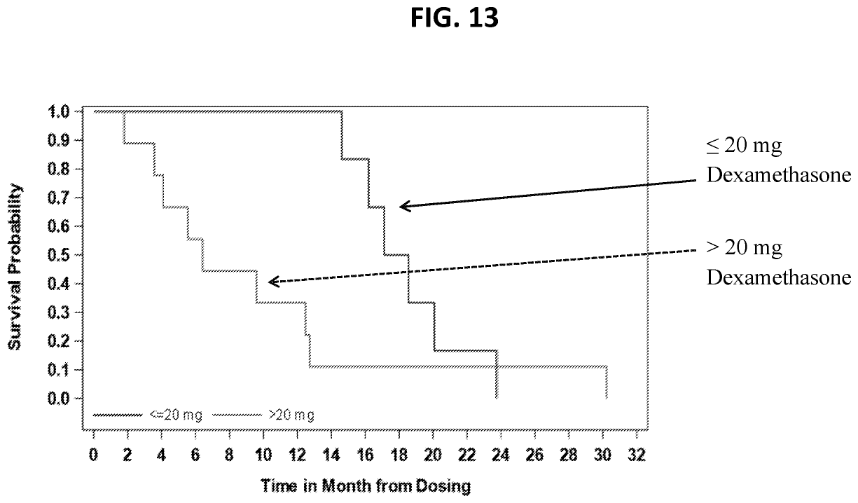

Rationale for Expansion Substudy: 31 subjects undergoing craniotomy were enrolled at four doses of veledimex in the Main Study, with 15 treated with 20 mg. mOS at this dose was 12.7 months with a mean follow up of 13.1 months. Ad hoc analysis of steroid use during active treatment in the Main Study showed a negative impact on mOS, as shown in FIG. 13, FIGS. 25A-25C, and Table 1, with median overall survival (mOS) increasing to 17.8 months in the 20 mg cohort for subjects that received a cumulative dose of <20 mg (less than or equal to 20 mg) of steroids during active dosing. These results led to the design for the “Expansion Substudy” (refer to FIG. 12B) to further investigate efficacy. Additional changes included requirement of a diagnosis of recurrent glioblastoma, no prior treatment with bevacizumab and no steroids for 4 weeks prior to the study entry. Additionally, subjects dosed at 20 mg V and having minimal cumulative steroid exposure (i.e., ≤20 mg (less than or equal to 20 mg) during active V dosing were observed to have an improved mOS (17.8 mos); refer to FIG. 24, FIGS. 25A-25B and FIG. 13 for further details. Approximately 65% of evaluated subjects treated received a cumulative dose of dexamethasone of <20 mg from Day 0-14. The observed safety profile exhibited acceptable results with cytokine release syndrome (CRS) characterized by flu-like symptoms with decreased white blood cell count, platelet count and/or increase in transaminases. All observed adverse reactions were reversable and manageable upon discontinuation of veledimex.
Methods (Expansion Substudy): An open label, single arm Phase 1 substudy evaluating safety and tolerability of local, inducible IL-12 expression in adult subjects with recurrent glioblastoma (rGBM) who were bevacizumab naïve (i.e., not previously treated with bevacizumab) and non-steroid dependent during the 4 weeks prior to Ad injection was subsequently commenced; refer to FIG. 12B (“Expansion Substudy”). Following standard-of-care resection Ad was administered by freehand intratumoral injection (at doses of 2×10¹¹ vp) with oral V (at 20 mg/dose) QD×15 doses from Days 0 to 14.
Results (Expansion Substudy): In the Expansion Substudy, Ad+V (with V at 20 mg/dose) increased serum IL-12 and downstream IFN-γ expression from a median baseline of 0.8 pg/mL IL-12 to 8.8 pg/mL IL-12 at Day 3; and, from a median baseline of 0 pg/mL IFN-γ to 8.6 pg/ml IFN-γ at Day 3. Similar trends in cytotoxic T cells, Tregs and peripheral immune cell activation were observed during dose escalation. Between median baseline and Day 14, cytotoxic T cells increased (CD3+CD8+ from 26% to 28%), Tregs decreased (FoxP3+ from 1.3% to 0.9%) with a resulting net activation of the immune system (CD8⁺/FoxP3⁺ ratio from 20 to 46). Median Overall Survival (mOS) was observed, in the study to date, as 12.7 months in subjects who received 20 mg V.

TABLE 1
Impact of Dexamethasone Use on Overall Survival
(ATI001-102; Main Study: 20 mg V Cohort)

| | Dexamethasone Use | | mOS | Lower | Upper | Mean | No. | No. |
|---|---|---|---|---|---|---|---|---|
| | (Days 0-14) | | (months) | bound | bound | F/U | Events | Censored |
| 20 mg veledimex | ≤20 | mg | 17.8 | 14.6 | 23.7 | 18.4 | 6 | 0 |
| with craniotomy | >20 | mg | 6.4 | 1.8 | 12.7 | 9.6 | 9 | 0 |

TABLE 2
Subject Characteristics

| | Main Study | | Expansion Substudy | |
|---|---|---|---|---|
| | V 20 mg Cohort | | V 20 mg Cohort | |
| Characteristic | (N = 15) | | (N = 36) | |
| Age [Yrs, Mean (Min, Max)] | 45.93 | (26, 68) | 51.5 | (21, 72) |
| Gender Male:Female | 10:5 | | 22:14 | |
| Recurrence (n) | | | | |
| 1^{st} | 4 | | 21 | |
| 2^{nd} | 5 | | 3 | |
| 3^{rd} or more | 6 | | 3 | |
| TBC | 0 | | 9 | |
| Prior Lines of Treatment (mean) | 2.2 | | 1.7 | |
| rGBM Grade (Study Entry) | | | | |
| Grade III (HGG) | 2 | | 0 | |
| Grade IV (Glioblastoma) | 13 | | 36 | |
| Performance Status: KPS (screening) | | | | |
| ≥90 | 9 | | 23 | |
| ≥70 and <90 | 6 | | 13 | |
| Veledimex Dosing Compliance (mean) | | | | |
| GBM Dosing - V QD (15days) | 84% | | 91.8% | |
| Cumulative Steroid Use | | | | |
| Days 0-14 (mg) (mean, range) | 60 | (0, 140) | 23.2 | (0, 166) |
| Percent receiving ≤20 mg steroids | 40% | (6/15) | 75% | (27/36) |
| during active dosing | | | | |

TABLE 3
Safety Results

| | Studies/Dose Cohorts | |
|---|---|---|
| | Main Study | Expansion Substudy |
| Adverse | 20 mg | 20 mg |
| Events | (N = 15) | (N = 36) |

Related ≥ Grade 3 AEs in ≥5% of Subjects

| | | |
|---|---|---|
| Lymphopenia | 2 (13%) | 2 (6%) |
| Thrombocytopenia | 2 (13%) | 0 |
| Leukopenia | 1 (7%) | 1 (3%) |
| Neutropenia | 1 (7%) | 1 (3%) |
| AST/ALT increased | 1 (7%) | 1 (3%) |
| Headache | 3 (20%) | 1 (3%) |
| Meningitis Aseptic | 1 (7%) | 0 |
| Hyponatremia | 2 (13%) | 0 |
| Amylase increased | 1 (7%) | 0 |

Related Serious Adverse Events (SAEs)

| | | |
|---|---|---|
| Pyrexia | 1 (7%) | 1 (3%) |
| Cytokine Release Syndrome | 2 (13%) | 0 |
| Thrombocytopenia | 2 (13%) | 0 |
| Neutropenia | 1 (7%) | 1 (3%) |
| Leukopenia | 1 (7%) | 0 |
| AST/ALT increased | 1 (7%) | 0 |
| Meningitis Aseptic | 1 (7%) | 0 |
| Mental Status Change | 0 | 1 (3%) |

Cytokine Release Syndrome (Ziopharm CRS Working Definition)

| | | |
|---|---|---|
| Grade 3 | 2 (13%) | 2 (6%) |

TABLE 4
Impact of Dexamethasone on Survival
Cumulative Steroids (Days 0-14)

| | | | | | Mean | Min | Max | |
|---|---|---|---|---|---|---|---|---|
| | | | | Median | Follow- | Follow- | Follow- | |
| # of | | # of Subjects | | Survival | up | up | up | 95% |
| Subjects | | Alive | | (Mos) | (Mos) | (Mos) | (Mos) | CI |
| ≤20 | mg | 27 | 23 | Not Yet | 3.3 | 1.0 | 7.1 | 2.6, |
| | | | | Reached | | | | 4.0 |
| >20 | mg | 9 | 8 | Not Yet | 5.0 | 1.9 | 7.5 | 3.6, |
| | | | | Reached | | | | 6.5 |

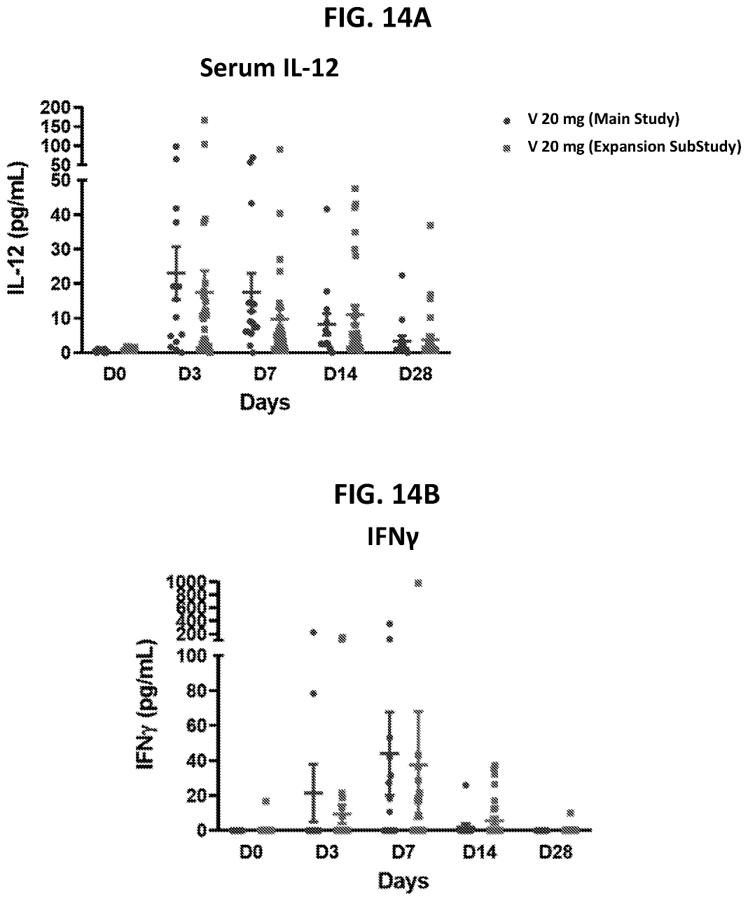

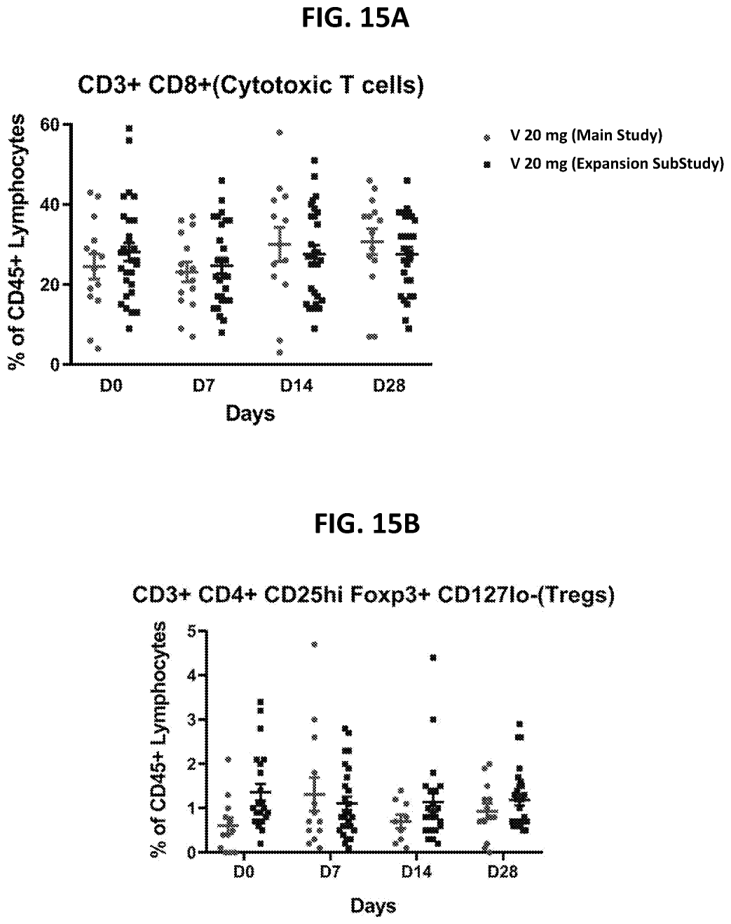

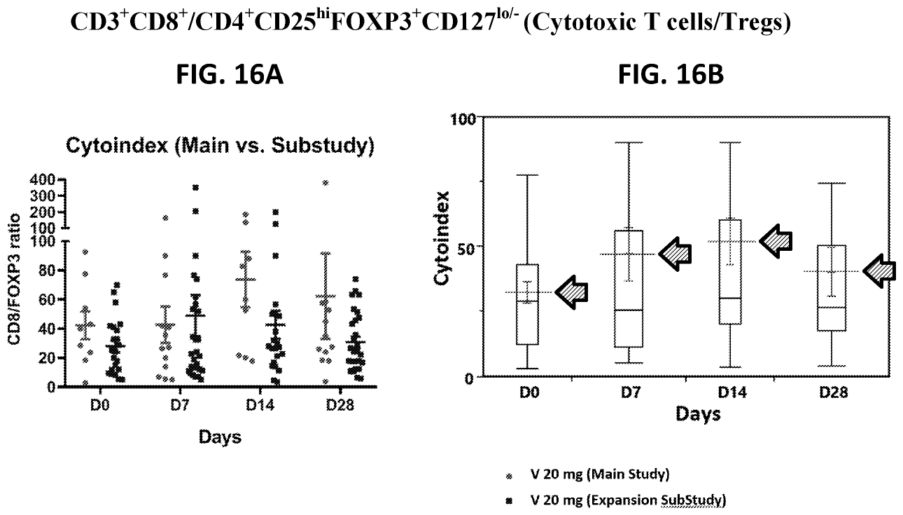

Conclusions: Plasma and tumor V peak plasma PK levels were dose dependent and resulted in production of IL-12 and downstream IFN-γ both detectable in serum (FIGS. 21A-21D). Serum recombinant IL-12 peaked at Day 3 with downstream production of endogenous serum IFN-γ peaking at Day 7 in both the Main Study and Expansion Substudy (FIGS. 21C and 21D for all V cohorts, and FIGS. 14A and 14B). Mean cytoindex increased from Day 0 to Day 7 then Day 14 before decreasing by Day 28 (Main Study and Substudy data combined) (FIGS. 16A and 16B). This peripheral immune response is consistent with the results seen in the Main Study and correlated with OS. Therefore, V crosses the blood-brain-barrier and regulates transcription of recombinant IL-12 from the adenoviral vector injected into the tumor, eliciting and sustaining an intra-tumoral immune response. Therefore V crosses the blood-brain-barrier and regulates transcription of recombinant IL-12 from the adenoviral vector injected into the tumor, eliciting and sustaining an intra-tumoral immune response. Drug-related toxicities in both the Main Study and Expansion Substudy were reversible upon discontinuation of veledimex with no drug related deaths. In the Expansion Substudy, the mOS has not yet been reached as of the 4 Jun. 2019 data cut-off for SNO 2019. A higher percentage of subjects in the Expansion Substudy (approximately 75% at the ASCO 2019 data cut-off 6 May 2019 and 65% at the SNO 2019 data cut-off of 4 Jun. 2019 vs 40% in the Main Study) received low-dose concurrent steroids (≤20 mg dexamethasone total, Days 0-14), which in the Main Study showed a trend towards improved OS (FIG. 25C). Local, regulated IL-12 production using Ad+V in subjects with rGBM rapidly and safely activates the immune system. Local, controlled IL-12 expression via administration of Ad+V in rGBM patients results in biological activity indicative of therapeutically desirable effects (see e.g., FIGS. 15A and 15B) and a favorable safety profile. Cytoindex, an emerging biomarker for enhanced peripheral cytotoxic immune response, increased following increases in peripheral IL-12 and IFN-γ concentrations (FIGS. 14A and 14B). As compared with Day 0, the mean cytoindex increased on Day 7 then Day 14 before decreasing by Day 28 in combined data from the Main Study and Expansion Substudy (FIGS. 16A and 16B). Subjects received less dexamethasone for prophylaxis or post procedure control edema during active dosing in the Expansion Substudy as compared with the Main Study (75% as of 6 May 2019 or 65% as of 4 Jun. 2019 vs 40% at ≤20 mg total). Lower/lowered use of steroids is associated with improved clinical outcome. See FIGS. 12-19 and FIGS. 20-26.

### Example 9—Administration of Ad-RTS-hIL-12 and Veledimex in Pediatric Subjects with Brain Tumors or DIPG

Background: Diffuse Intrinsic Pontine Glioma (DIPG) is an unmet need having a significant mortality among children with a mOS of 9 months and <10% survival rate at 2 years. Ad-RTS-hIL-12 (Ad) is a recombinant, adenoviral-delivered, gene therapy for expression of interleukin-12 (IL-12) under the control of an orally administered activator ligand, veledimex (V), acting in concert with a ligand-inducible gene switch (also referred to as “RTS®”). Local expression of IL-12 results in an influx of cytotoxic T cells into the tumor and subsequent tumor cell death. Nonclinical studies in a GL-261 medullary glioma orthotopic mouse model where Ad was administered at 5×10⁹ vp with V at doses of 3-30 mg/m² and QDx14 demonstrated a dose-related increase in survival. At Day 85 Ad+V (with V at 10 mg/m²; a human equivalent dose 20 mg) 67% of 12 animals were alive and devoid of clinical signs compared to a mOS of 16 days for vehicle and mOS of 25 days for temozolomide; indicative of therapeutically beneficial effects for Ad+V monotherapy in treating DIPG.

Methods: A Phase 1 dose escalation study to determine safety and tolerability of Ad+V in pediatric brain tumor subjects is undertaken. The study includes two “Arms”: Arm 1 consists of pediatric brain tumor subjects scheduled to receive standard-of-care craniotomy and tumor resection, excluding subjects with diffuse intrinsic pontine glioma (DIPG). Arm 2 consists of subjects with DIPG and post prior standard focal radiotherapy. Arm 1 subjects receive one dose of V prior to tumor resection, then following intraoperative intratumoral injection of Ad (2×10¹¹ vp), are administered oral V for 14 additional days. Arm 2 subjects receive a single Ad stereotactic injection followed by oral V for 14 days. Both arms receive body surface area (BSA)-adjusted, escalating doses of V at 10 or 20 mg PO. Endpoint measurements include assessment of safety as determined by the adverse event (AE) rate and the occurrence of DLTs analyzed by cohort, pharmacokinetics of V, V tumor to blood ratio, immunologic and biomarker characterization of the immune response elicited, and investigator assessment of objective response rate, progression free survival, and overall survival.

### Example 10—Administration of Ad-RTS-hIL-12 Plus Veledimex in Combination with a PD-1 Inhibitor in Subjects with Recurrent or Progressive Glioblastoma

Summary: Ad-RTS-hIL-12 (Ad) is a recombinant, adenoviral-delivered, gene therapy for expression of interleukin-12 (IL-12) under the control of an orally administered activator ligand, veledimex (V), acting in concert with a ligand-inducible gene switch (also referred to as “RTS®”). Intratumoral administration of Ad+V elicits tumoral and local tissue influx of cytotoxic T cells resulting in targeted tumor cell cytotoxicity. Nivolumab (“nivo”) is a (receptor antagonist) antibody that binds Programmed cell Death protein-1 (anti-PD-1) and thereby enhances activity of T cells; i.e., for increased T cell anti-tumor engagement of cancer cells and tumors/tumor cells. In GL-261 orthotopic mouse, a supra additive effect on survival was observed with combination therapy of Ad+V (30 mg/m²) QDx14 days and anti-PD-1 with all animals surviving vs 63% for Ad+V vs 40% for anti-PD-1 alone (FIG. 6).

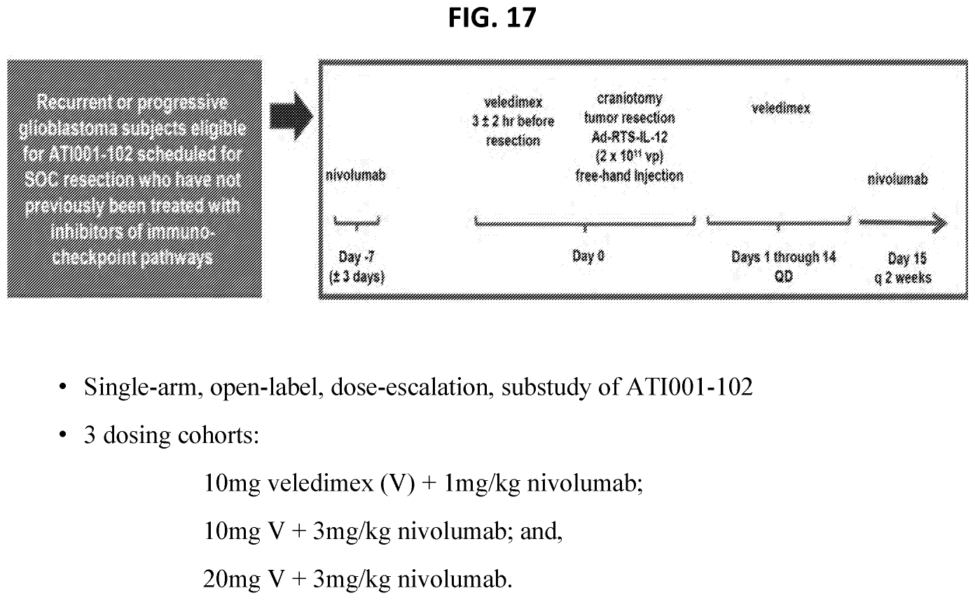

Methods: An open label, dose escalation Phase 1 study evaluating safety and tolerability of local, inducible IL-12 expression in combination with nivolumab in adult subjects with recurrent glioblastoma (rGBM) was commenced. Ad was administered by intratumoral injection (using a dose of 2×10¹¹ vp (2e11 vp)) along with daily veledimex (V) (at doses of 10-20 mg) QDx15 PO with nivolumab intravenous infusions (at doses of 1-3 mg/kg) at Day (−)7 (i.e., 7 days prior to administration of Ad+V), at Day 15 (after administration of Ad+V), then Q2W (i.e., once every 2-weeks). See Table 5 and FIG. 17.

TABLE 5
Subject characteristics across three cohorts.

| | Cohort 1: Ad + V | Cohort 2: Ad + V | Cohort 3: Ad + V |
|---|---|---|---|
| | (10 mg) | (10 mg) | (20 mg) |
| | Nivolumab (1 mg/kg) | Nivolumab (3 mg/kg) | Nivolumab (3 mg/kg) |
| | N = 3 | N = 3 | N = 2 |
| Gender (M:F) | 1:2 | 1:2 | 1:1 |
| Age (mean, range) | 43.0 (30, 63) | 59.4 (52, 66) | 61.4 (47, 76) |
| Performance (KPS, | | | |
| screening) | | | |
| 70-80 | 0 | 0 | 1 |
| 90-100 | 3 | 3 | 1 |
| Recurrences (mean, | 1.7 (1, 3) | 1 (1, 1) | 1.5 (1, 2) |
| range) | | | |
| Lines of Therapy | 1.3 (1, 2) | 1 (1, 1) | 2 (1, 3) |
| (mean, range) | | | |
| IDH Mutation Status | 1:2 | 0:3 | 1:1 |
| Mutated: Wild- | | | |
| Type | | | |
| V dosing compliance | 97.8 | 100 | — |
| (%) (mean) | | | |
| Steroid Use (mg) | 64.7 (0, 116) | 3.3 (0, 10) | — |
| (mean, range) | | | |

TABLE 6
Safety Results

| | Adverse Event | | | | | | |
|---|---|---|---|---|---|---|---|
| | | | | | | | ATI001-102 |
| | | | | | | | Main Study |
| | Cohort 1: | | Cohort 2: | | Cohort 3: | | Ad + V 10 and |
| | Ad + V (10 mg) | | Ad + V (10 mg) | | Ad + V (20 mg) | | 20 mg with |
| | Nivolumab (1 mg/kg) | | Nivolumab (3 mg/kg) | | Nivolumab (3 mg/kg) | | Craniotomy, |
| Dose cohort | N = 3 | | N = 3 | | N = 2 | | N = 21 |
| Relatedness | Ad + V | N | Ad + V | N | Ad + V | N | Ad + V |

Related ≥ Grade 3 AEs

| | | | | | | | |
|---|---|---|---|---|---|---|---|
| Lymphocyte count | 1 (33%) | 0 | 1 (33%)* | 1 (33%)* | 1 (50%) | 0 | 3 (20%) |
| Decreased | | | | | | | |
| ALT increased | 0 | 0 | 1 (33%) | 0 | 0 | 0 | 3 (20%) |
| Brain oedema | 0 | 1 (33%) | 0 | 0 | 0 | 0 | 0 |
| (cerebral edema) | | | | | | | |
| Lipase increased | 0 | 0 | 0 | 1 (33%) | 0 | 0 | 0 |

Cytokine Release Syndrome
(ZIOPHARM CRS Working Definition)

| | | | | |
|---|---|---|---|---|
| Grade 3 | 0 | 0 | 0 | 2 (10%) |

*One ≥ Grade 3 AE (Lymphocyte count decreased) was considered related to both Ad + V and N
No DLTs
One SAE of brain oedema (cerebral edema) was considered related to nivolumab alone
No SAEs were considered related to Ad + V or the combination
No related Grade 4 or fatal AEs
No clinically significant overlapping toxicities

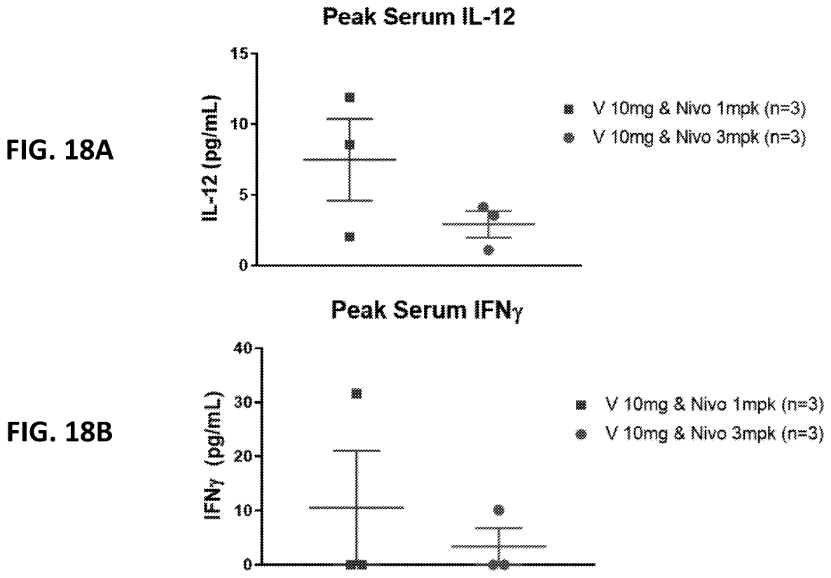

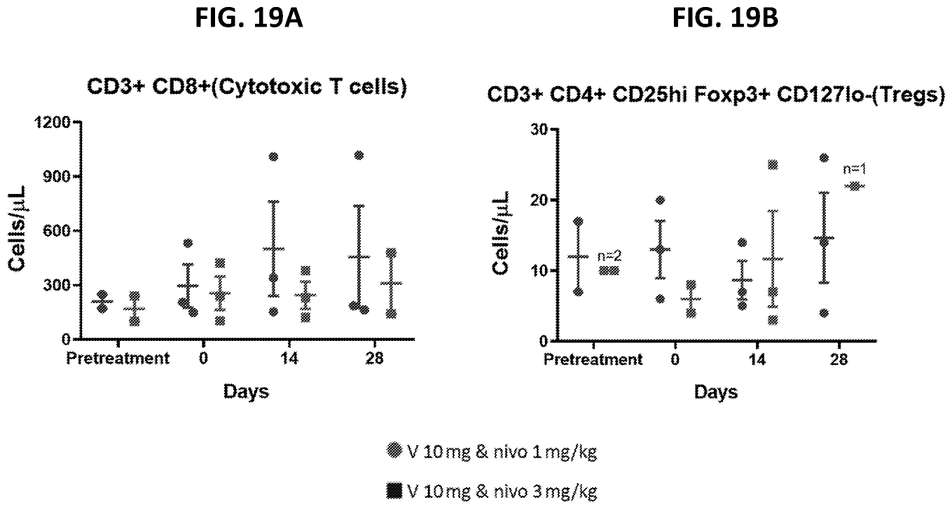

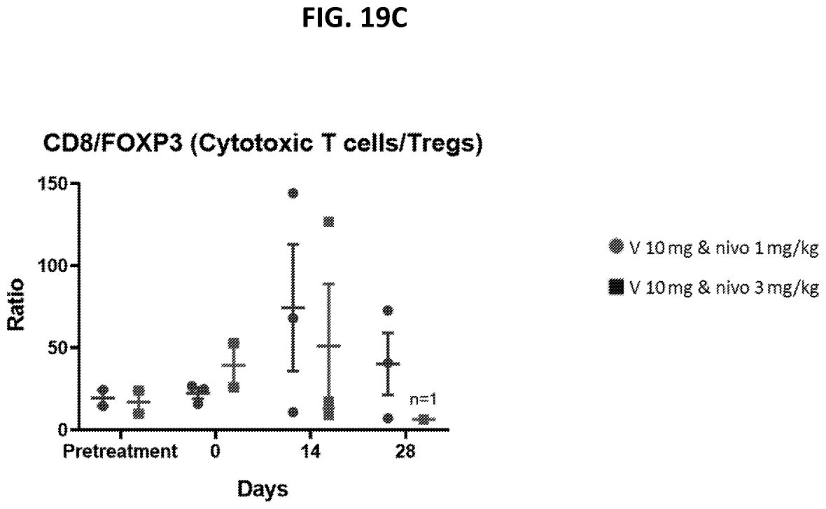

Results: Nivolumab alone did not alter peripheral IL-12 levels (median baseline 0.9 pg/ml vs Day 0 at 1.0 pg/ml). Ad+V increased peripheral IL-12 to 5.5 pg/ml on Day 3. Nivo alone increased peripheral cytotoxic T cells (CD3+CD8+ median baseline 23% vs Day 0 at 26%) with Ad+V increasing CD3⁺CD8⁺ further to 31% at Day 14. Nivolumab alone decreased Tregs (FoxP3 baseline 1.5% vs Day 0 at 0.8%) with Ad+V further decreasing Tregs to 0.3% (Day 14). Combination therapy resulted in net activation of the immune system (CD8⁺/FoxP3⁺ ratio baseline 15 vs 29 (Day 0) vs 80 (Day 14)). Interim safety data showed a similar adverse events (AEs) profile compared to monotherapy with Ad+V during the V dosing period. AEs in the subsequent treatment period with nivolumab were consistent with those previously reported. Adverse reactions observed have been manageable and reversible. No synergism of toxicities was observed. See FIGS. 18A, 18B, 19A, and 19B.

TABLE 7A
IL-12 serum cytokine levels across 10 mg veledimex cohorts.

| | Baseline | Pre - Ad + V | Peak | | |
|---|---|---|---|---|---|
| IL-12 (pg/mL) | Mean ± SD | Mean ± SD | Mean ± SD | Min | Max |
| V 10 mg & Nivo 1 mg/kg | 1.2 ± 0.7 | 0.9 ± 0.3 | 7.5 ± 5.0 | 2.1 | 11.9 |
| V 10 mg & Nivo 3 mg/kg | 0.7 ± 0.2 | 1.1 ± 0.7 | 2.9 ± 1.6 | 1.1 | 4.1 |

TABLE 7B
Interferon-gamma levels across 10 mg veledimex cohorts.

| | Baseline | Pre - Ad + V | Peak | | |
|---|---|---|---|---|---|
| IFNγ (pg/mL) | Mean ± SD | Mean ± SD | Mean ± SD | Min | Max |
| V 10 mg & Nivo 1 mg/kg | 3.6 ± 6.2 | 2.8 ± 4.8 | 10.5 ± 18.2 | 0 | 31.6 |
| V 10 mg & Nivo 3 mg/kg | 0.0 ± 0.0 | 0.0 ± 0.0 | 3.4 ± 5.8 | 0 | 10.1 |

TABLE 8
Immune cell marker profiles across 10 mg veledimex cohorts.

| | V 10 mg & Nivo 1 mg/kg | | | V 10 mg & Nivo 3 mg/kg | | |
|---|---|---|---|---|---|---|
| Cytoindex | Mean | SD | N | Mean | SD | N |
| Pre-treatment | 19.5 | 7.0 | 2 | 17.0 | 9.9 | 2 |
| Day 0 | 22.4 | 5.8 | 3 | 39.4 | 19.0 | 2 |
| Day 14 | 74.4 | 66.9 | 3 | 51.1 | 65.5 | 3 |
| Day 28 | 40.2 | 32.8 | 3 | 6.5 | NA | 1 |

Conclusion: Enrollment is ongoing in the 3+3 dose escalation study, with regulatory pauses required between patients and cohorts. Mean follow-up is 4.5 months (min 0.4 months for most recently enrolled patient, max 10.1 months for the first enrolled patient). 66% received low-dose concurrent steroids (≤20 mg dexamethasone total, Days 0-14). Pre-dosing with nivolumab did not have an impact on cytokine levels prior to Ad+V. Increased measurement of recombinant IL-12 and endogenous IFN-γ in the serum following initiation of Ad+V (Controlled IL-12), which is consistent with previously reported data of Ad+V monotherapy. Cytoindex, an emerging biomarker for effects of IL-12, showed activation of the immune system. No significant overlapping toxicities were identified. Local, controlled IL-12 expression using Ad+V in combination with anti-PD-1 inhibitors in adult patients with rGBM results in biological activity indicative of therapeutically desirable effects and favorable safety profile.

Embodiments (E) of the invention include:
E1. A method of treating a subject having a cancerous tumor and/or preventing development of cancerous tumor in the subject, the method comprising:
A) administering to the subject an effective amount of a vector comprising a polynucleotide encoding an ecdysone receptor-based gene switch, wherein the polynucleotide comprises:
(1) at least one transcription factor sequence which is operably linked to a promoter, wherein the at least one transcription factor sequence encodes a ligand-dependent transcription factor, and
(2) a first polynucleotide encoding a first polypeptide which is at least 85% identical to wild type IL-12 p40, a second polynucleotide encoding a second polypeptide which is at least 85% identical to wild type IL-12 p35, wherein the first polynucleotide and the second polynucleotide are operably linked to a promoter which is activated by the ligand-dependent transcription factor;
B) administering to the subject an effective amount of a diacylhydrazine ligand that activates the ligand-dependent transcription factor; and
C) administering to the subject an effective amount of an immune checkpoint inhibitor.
E2. The method of E1, wherein a first dose of the diacylhydrazine ligand (e.g, veledimex) is administered to the subject during a time period from about 24 hours before administration of the vector to about 36 hours following administration of the vector.
E3. The method of E1 or E2, wherein the diacylhydrazine ligand is administered daily for a period of 3 to 28 days.
E4. The method of any one of E1-E3, wherein the diacylhydrazine ligand is administered daily for a period of 14 days.
E5. The method of any one of E1-E4, wherein the diacylhydrazine ligand is administered daily at a dose of about 5 to about 80 mg.
E6. The method of any one of E1-E5, wherein the diacylhydrazine ligand is administered daily at a dose of about 10 mg.
E7. The method of any one of E1-E5, wherein the diacylhydrazine ligand is administered daily at a dose of about 20 mg.
E8. The method of any one of E1-E7, wherein the diacylhydrazine ligand is administered orally.
E9. The method of any one of E1-E8, wherein the diacylhydrazine ligand is (R)—N′-(3,5-dimethylbenzoyl)-N′-(2,2-dimethylhexan-3-yl)-2-ethyl-3-methoxybenzohydrazide.
E10. The method of any one of E1-E9, wherein the vector is an adenoviral vector.
E11. The method of any one of E1-E10, wherein the vector is an adenoviral serotype 5 vector.
E12. The method of any one of E1-E11, wherein the vector is a replication-deficient adenoviral vector.
E13. The method of any one of E1-E12, wherein the vector is administered intratumorally.
E14. The method of any one of E1-E13, wherein the vector is administered at a dose of about 0.01×10¹¹ viral particles to about 20×10¹¹ viral particles.
E15. The method of any one of E1-E14, wherein the vector is administered at a dose of about 2×10¹¹ viral particles.
E16. The method of any one of E1-E14, wherein the vector is administered at a dose of about 3×10¹¹ viral particles.
E17. The method of any one of E1-E16, wherein the wild type IL-12 p40 is wild type human IL-12 p40, and the wild type IL-12 p35 is wild type human IL-12 p35.
E18. The method of any one of E1-E16, wherein the wild type IL-12 p40 is wild type mouse IL-12 p40, and the wild type IL-12 p35 is wild type mouse IL-12 p35.
E19. The method of any one of E1-E18, wherein the first polynucleotide and the second polynucleotide is joined by a linker.
E20. The method of E19, wherein the linker is a ribosome entry site (IRES) sequence.
E21. The method of any one of E1-E20, wherein the immune checkpoint inhibitor is administered once every 1, 2, 3, 4, 5, or 6 weeks starting from the day on which the first dose is administered.
E22. The method of any one of E1-E20, wherein the immune checkpoint inhibitor is administered once every 1, 2, 3, 4, 5, or 6 weeks starting from the day on which the second dose is administered.
E23. The method of any one of E1-E20, wherein a first dose of the immune checkpoint inhibitor is administered to the subject at about 5-10 days before administration of the vector, and wherein a second dose of the immune checkpoint inhibitor is administered to the subject at about 7 to 28 days after administration of the vector.
E24. The method of E23, wherein the first dose of the immune checkpoint inhibitor is administered to the subject at about 7 days before administration of the vector
E25. The method of E23, wherein the second dose of the immune checkpoint inhibitor is administered to the subject at about 15 days after administration of the vector.
E25a. The method of E23, wherein the second dose of the immune checkpoint inhibitor is administered to the subject concomitant with administration of the vector.
E25b. The method of E23, wherein the additional doses of the immune checkpoint inhibitor are administered to the subject by sequential dosing following administration of the vector.
E26. The method of any one of E23-E25, wherein the immune checkpoint inhibitor is administered once every two weeks or once every three weeks starting from the day on which the second dose is administered.
E27. The method of any one of E1-E20, wherein a first dose of the immune checkpoint inhibitor is administered to the subject in a time period from about 24 hours before administration of the vector to about 36 hours following administration of the vector.
E28. The method of any one of E1-E20, wherein a first dose of the immune checkpoint inhibitor is administered to the subject at about 1 to 28 days after administration of the vector.
E29. The method of any one of E1-E28, wherein the immune checkpoint inhibitor is a PD-1 antagonist, a PD-L1 antagonist, a PD-L2 antagonist, a CTLA-4 antagonist, a CD137 antagonist, a CD80 antagonist, a CD86 antagonist, a KIR antagonist, a Tim-3 antagonist, a LAG3 antagonist, a T-cell immunoreceptor with Ig and ITIM domains (TIGIT) antagonist, a CD96 antagonist, or an IDO1 antagonist.
E29a. The method of any one of E1-E28, wherein the immune modulator to be combined with Ad+V therapy is a cytokine other than IL-12; a CD25 antagonist; a B- and T-cell attenuator (BTLA); a targetable member of the tumor necrosis (TNF) superfamily including but not limited to CD40 and CD40L, OX40, 4-1BB (CD137) and 4-1BBL (CD137L), Glucocorticoid-induced TNFR family related protein (GITR), GITR ligand (GITRL), CD27 antagonist; a tumor associated protein such as a CD20 antagonist; transforming growth factor-beta (TGF-0); T-cell immunoreceptor with Ig and ITIM domains; T-cell co-stimulation (e.g., o-stimulatory receptors are members of the tumor necrosis factor receptor (TNFR) family); a CD276 (B7-H3) antagonist, a VTCN1 (B7-H4) antagonist, an A2AR antagonist, a BTLA antagonist, a NOX2 antagonist, a VISTA antagonist, a SIGLEC7 antagonist, a SIGLEC9 antagonist; T cell-inducing vaccine; a dendritic cell-inducing vaccine; an NK cell-inducing vaccine; an administration of T cells by infusion; and an administration of NK cells by infusion.
E29b. The method of any one of E1-E28, wherein the immune modulator to be combined with Ad+V therapy is of a different therapeutic modality such as chemotherapy, radiation including SRS or surgery, particularly if that modality may help elicit production of neoantigens.
E30. The method of E29, wherein the PD-1 antagonist is nivolumab (MDX 1106), pembrolizumab (MK-3475), pidilizumab (CT-011), MEDI-0680 (AMP-514), PDR001, cemiplimab-rwlc (REGN2810), AMP-224, STI-A1110, AUNP-12, or BGB-A317.
E31. The method of E29 or E30, wherein the PD-1 antagonist is nivolumab, and the PD-1 antagonist is administered at a dose of about 0.5 mg/kg to 5 mg/kg.
E32. The method of any one of E29-E31, wherein the PD-1 antagonist is nivolumab, and the PD-1 antagonist is administered at a dose of about 1 mg/kg.
E33. The method of any one of E29-E31, wherein the PD-1 antagonist is nivolumab, and the PD-1 antagonist is administered at a dose of about 3 mg/kg.
E33a. The method of any one of E29-E31, wherein the PD-1 antagonist is nivolumab, and the PD-1 antagonist is administered at a flat dose of about 240 mg every two weeks.
E33b. The method of any one of E29-E31, wherein the PD-1 antagonist is nivolumab, and the PD-1 antagonist is administered at a flat dose of about 480 mg every four weeks.
E34. The method of E29 or E30, wherein the PD-1 antagonist is pembrolizumab, and the PD-1 antagonist is administered at a dose of about 50 mg to about 300 mg.
E35. The method of E29 or E30, wherein the PD-1 antagonist is pidilizumab, and the PD-1 antagonist is administered at a dose of about 0.5 mg/kg to about 3 mg/kg.
E36. The method of E29 or E30, wherein the PD-1 antagonist is MEDI-0680, and the PD-1 antagonist is administered at a dose of about 5 mg/kg to about 40 mg/kg.
E37. The method of E29 or E30, wherein the PD-1 antagonist is PDR001, and the PD-1 antagonist is administered at a dose of about 100 mg to about 800 mg.
E38. The method of E29 or E30, wherein the PD-1 antagonist is cemiplimab-rwlc (REGN2810), and the PD-1 antagonist is administered at a dose of about 0.5 mg/kg to about 6 mg/kg.
E39. The method of E29 or E30, wherein the PD-1 antagonist is BGB-A317, and the PD-1 antagonist is administered at a dose of about 0.5 mg/kg to about 6 mg/kg.
E40. The method of E29 or E30, wherein the PD-1 antagonist is AMP-224, and the PD-1 antagonist is administered at a dose of about 2 mg/kg to about 20 mg/kg.
E41. The method of E29, wherein the PD-L1 antagonist is atezolizumab (RG7446, MPDL3280A), MEDI4736 (durvalumab), BMS-936559 (MDX-1105), avelumab (MSB0010718C), or KD033 (Kadmon).
E42. The method of E29 or E41, wherein the PD-L1 antagonist is atezolizumab, and the PD-L1 antagonist is administered at a dose of about 500 mg to about 2000 mg.
E43. The method of E29 or E41, wherein the PD-L1 antagonist is MEDI4736, and the PD-L1 antagonist is administered at a dose of about 500 mg to about 2000 mg.
E44. The method of E29 or E41, wherein the PD-L1 antagonist is BMS-936559, and the PD-L1 antagonist is administered at a dose of about 0.1 mg/kg to about 10 mg/kg.
E45. The method of E29 or E41, wherein the PD-L1 antagonist is avelumab, and the PD-L1 antagonist is administered at a dose of about 5 mg/kg to about 20 mg/kg.
E46. The method of E29, wherein the CTLA-4 antagonist is ipilimumab (YERVOY), tremelimumab (CP-675,206), or KAHR-102.
E47. The method of E29 or E46, wherein the CTLA-4 antagonist is ipilimumab, and the CTLA-4 antagonist is administered at a dose of about 0.5 mg/kg to about 10 mg/kg.
E48. The method of E29 or E46, wherein the CTLA-4 antagonist is tremelimumab, and the CTLA-4 antagonist is administered at a dose of about 1 mg/kg to about 20 mg/kg.
E49. The method of E29 or E46, wherein the CTLA-4 antagonist is KAHR-102, and CTLA-4 antagonist is administered at a dose of about 1 pg/kg to about 20 pg/kg.
E50. The method of E29, wherein the CD137 antagonist is PRS-343, PF-2566 (PF-05082566), or urelumab (BMS-663513).
E51. The method of E29 or E50, wherein the CD137 antagonist is PRS-343, and the CD137 antagonist is administered at a dose of about 0.01 pg/kg to about 1000 mg/kg.
E52. The method of E29 or E50, wherein the CD137 antagonist is PF-2566, and the CD137 antagonist is administered at a dose of about 0.1 mg/kg to about 10 mg/kg.
E53. The method of E29 or E50, wherein the CD137 antagonist is urelumab, and CD137 antagonist is administered at a dose of about 0.1 mg/kg to about 20 mg/kg.
E54. The method of E29, wherein the LAG3 antagonist is IMP701, IMP731, BMS-986016, LAG525, GSK2831781, or IMP321.
E55. The method of E29 or E54, wherein the LAG3 antagonist is IMP701, IMP731, IMP321, or LAG525, and the LAG3 antagonist is administered at a dose of about 0.01 mg/kg to about 200 mg/kg.
E56. The method of E29 or E54, wherein the LAG3 antagonist is GSK2831781, and the LAG3 antagonist is administered at a dose of about 20 mg/kg to about 500 mg/kg.
E57. The method of E29 or E54, wherein the LAG3 antagonist is BMS-986016, and LAG3 antagonist is administered at a dose of about 20 mg to about 200 mg.
E58. The method of E29, wherein the KIR antagonist is lirilumab (IPH2102).
E59. The method of E29 or E58, wherein the KIR antagonist is administered at a dose of about 0.01 mg/kg to about 20 mg/kg.
E60. The method of any one of E1-E59, wherein the cancerous tumor is a solid tumor.
E61. The method of any one of E1-E60, wherein the cancerous tumor is a primary, progressive or recurrent tumor, including but not limited to the following types: glioma tumor, renal cancer tumor, an ovarian cancer tumor, a head and neck cancer tumor, a liver cancer tumor, a pancreatic cancer tumor, a gastric cancer tumor, an esophageal cancer tumor, a urothelial cell (bladder, ureter, or renal pelvis) cancer tumor, a urogenital (cervical, endometrial or penile) cancer tumor, a thyroid cancer tumor, or a prostate cancer tumor.
E61a. The method of any one of E1-E60, wherein the cancerous tumor is a primary, progressive or recurrent tumor, including but not limited to the following types: a breast cancer tumor, a melanoma tumor, a colon cancer tumor, a lung cancer tumor, or a squamous cell tumor.
E61b. The method of any one of E1-E61a, wherein the cancerous tumor is a tumor that is metastatic to the brain.
E62. The method of any one of E1-E61, wherein the subject has not been previously treated with an immune checkpoint inhibitor or other agents specifically targeting T cells, wherein the immune checkpoint inhibitor or agent specifically targeting T cells includes but is not limited to a PD-1 antagonist, a PD-L1 antagonist, a PD-L2 antagonist, a CTLA-4 antagonist, a CD137 antagonist, a CD80 antagonist, a CD86 an IDO1 antagonist, a KIR antagonist, a TIM-3 antagonist, a LAG3 antagonist, a TIGIT antagonist, a CD96 antagonist, a CD276 (B7-H3) antagonist, a VTCN1 (B7-H4) antagonist, an A2AR antagonist, a BTLA antagonist, a NOX2 antagonist, a VISTA antagonist, a SIGLEC7 antagonist, a SIGLEC9 antagonist. or an IDO1 antagonist.
E63. The method of any one of E1-E62, wherein the subject is an adult human.
E64. The method of any one of E1-E62, wherein the subject is a pediatric human.
E65. The method of any one of E1-E64, wherein the method produces an abscopal effect in the subject.
E66. A method of treating a subject having brain cancer, the method comprising administering to the subject a composition comprising a replication-deficient adenoviral vector,
   wherein the subject has previously been administered, has concurrently been administered, or will further be administered an immune checkpoint inhibitor,
   wherein the adenoviral vector comprises polynucleotide sequences encoding an ecdysone receptor-based polypeptide or polypeptides,
   wherein said ecdysone receptor-based polypeptide or polypeptides function as ligand-inducible (ligand activated) transcription factors,
   wherein the adenoviral vector further comprises a promoter polynucleotide sequence capable of being activated by said ligand-inducible transcription factors,
   wherein said ligand-inducible transcription factors activate transcription in response to contact with, or exposure to, diacylhydrazine ligands,
   wherein the adenoviral vector further comprises polynucleotide sequences encoding interleukin-12 (IL-12), wherein IL-12 comprises one or both of IL-12 p35 and p40 polypeptide subunits or is a biologically-active single chain IL-12 polypeptide,
   wherein polynucleotides encoding IL-12 are operably linked to the promoter polynucleotide sequence such that administration of a diacylhydrazine ligand to the subject is capable of inducing production of IL-12 in the subject.
E67. A method of treating a subject having brain cancer, the method comprising administering to the subject one or more immune modulators,
   wherein the subject has previously been administered, has concurrently been administered, or will further be administered a replication-deficient adenoviral vector,
   wherein the subject has previously been administered, has concurrently been administered, or will further be administered a diacylhydrazine ligand,
   wherein the adenoviral vector comprises polynucleotide sequences encoding an ecdysone receptor-based polypeptide or polypeptides,
   wherein said ecdysone receptor-based polypeptide or polypeptides function as ligand-inducible (ligand activated) transcription factors,
   wherein the adenoviral vector further comprises a promoter polynucleotide sequence capable of being activated by said ligand-inducible transcription factors,
   wherein said ligand-inducible transcription factors activate transcription in response to contact with, or exposure to, diacylhydrazine ligands,
   wherein the adenoviral vector further comprises polynucleotide sequences encoding interleukin-12 (IL-12),
   wherein IL-12 comprises one or both of IL-12 p35 and p40 polypeptide subunits or is a biologically-active single chain IL-12 polypeptide,
   wherein polynucleotides encoding IL-12 are operably linked to the promoter polynucleotide sequence such that administration of a diacylhydrazine ligand to the subject is capable of inducing production of IL-12 in the subject.
E68. A method of treating a subject having brain cancer, the method comprising administering to the subject a diacylhydrazine ligand,
   wherein the subject has previously been administered, has concurrently been administered, or will further be administered a replication-deficient adenoviral vector,
   wherein the subject has previously been administered, has concurrently been administered, or will further be administered an immune checkpoint inhibitor.
   wherein the adenoviral vector comprises polynucleotide sequences encoding an ecdysone receptor-based polypeptide or polypeptides,
   wherein said ecdysone receptor-based polypeptide or polypeptides function as ligand-inducible (ligand activated) transcription factors,
   wherein the adenoviral vector further comprises a promoter polynucleotide sequence capable of being activated by said ligand-inducible transcription factors,
   wherein said ligand-inducible transcription factors activate transcription in response to contact with, or exposure to, diacylhydrazine ligands,
   wherein the adenoviral vector further comprises polynucleotide sequences encoding interleukin-12 (IL-12),
   wherein IL-12 comprises one or both of IL-12 p35 and p40 polypeptide subunits or is a biologically-active single chain IL-12 polypeptide,
   wherein polynucleotides encoding IL-12 are operably linked to the promoter polynucleotide sequence such that administration of a diacylhydrazine ligand to the subject is capable of inducing production of IL-12 in the subject.
E69. The method of any one of E66-E68, wherein the subject is a human 18 years or older or a human under 18 years old.
E70. The method of any one of E66-E68, wherein the brain cancer is glioma, glioblastoma, recurrent glioblastoma, progressive glioblastoma, diffuse intrinsic pontine glioma (DIPG), or other diffuse midline gliomas (e.g., thalamus, brain stem or spinal cord).
E71. The method of any one of E66-68, wherein the replication deficient adenoviral vector was derived from a type 5 adenovirus genome.
E72. The method of any one of E1-E71, wherein the adenovirus is administered intratumorally.
E73. The method of E72, wherein the adenovirus is administered intratumorally by stereotactic targeting and delivery.
E74. The method of any one of E66-E68, wherein the diacylhydrazine ligand is veledimex.
E75. The method of E74, wherein any one or more administrations of veledimex are orally administered at a dose of 5 mg, 10 mg, 20 mg, 40 mg, 50 mg, 80 mg, 100 mg, 120 mg or 150 mg.
E76. The method of any one of E66-E68, wherein the ecdysone receptor-based polypeptides comprise herpes virus VP16 transactivation domain polypeptide sequences, chimeric mammalian retinoid X receptor (RxR) and insect ultraspiracle (USP) polypeptide sequences, Gal4 DNA binding domain polypeptide sequences, and amino acid substitution-mutated ecdysone receptor ligand binding domain polypeptides derived from spruce budworm Choristoneura fumiferana.
E77. The method of any one of E66-E68, wherein the immune checkpoint inhibitor is a PD-1 antagonist, a PD-L1 antagonist, a PD-L2 antagonist, a CTLA-4 antagonist, a CD137 antagonist, a CD80 antagonist, a CD86 antagonist, a KIR antagonist, a Tim-3 antagonist, a LAG3 antagonist, a TIGIT antagonist, a CD20 antagonist, a CD96 antagonist, or an IDO1 antagonist.
E78. The method of any one of E66-E68, wherein the immune checkpoint inhibitor is nivolumab (MDX 1106), pembrolizumab (MK-3475), pidilizumab (CT-011), MEDI-0680 (AMP-514), PDR001, cemiplimab-rwlc (REGN2810), AMP-224, STI-A1110, AUNP-12, or BGB-A317.
E79. The method in any one of E1-E78, wherein the subject has not been administered corticosteroids prior to the method of treatment in any one of E1-76.
E80. The method in any one of E1-E78, wherein administration of corticosteroids to the subject has been ceased or reduced prior to the method of treatment in any one of E1-76.
E81. The method in any one of E1-E78, wherein administration of corticosteroids to the subject is reduced or ceased during the method of treatment in any one of E1-76.
E82. The method in any one of E77-E81, wherein the corticosteroid is dexamethasone.
E83. The method in any one of E1-E82, wherein the method of treatment results in enhanced or increased recruitment of T cells into a tumor.
E84. The method of E83, wherein T cell activity in the tumor is improved or enhanced by reduction of expression or cell-surface presentation of immune checkpoint proteins or immune checkpoint activators.
E85. The method in any one of E1-E84, wherein veledimex is administered in an escalating dose.
E86. The method of E85, wherein the first dose is 10 mg and a subsequent dose or doses is, or are, greater than 10 mg.
E87. The method of E85, wherein the first dose is 10 mg and one or more subsequent doses is, or are, 20 mg.
E88. The method of E85, wherein the first dose is 10 mg and one or more subsequent doses is, or are, 30 mg, 40 mg, 50 mg, 80 mg, 100 mg, 120 mg or 150 mg.
E89. The method of any of E1-E88, wherein the immune checkpoint inhibitor is administered intravenously at 1 mg/kg or 3 mg/kg.
E90. The method of E89, wherein the immune checkpoint inhibitor is an anti-PD-1 antibody.
E91. The method of E90, wherein the PD-1-specific antibody is nivolumab.
E92. The method of E91, wherein nivolumab is OPDIVO© PD-1-specific antibody.
E93. The method of any of E89-E92, wherein the immune checkpoint inhibitor is administered one week before tumor resection.
E94. The method of E93, wherein the immune checkpoint inhibitor is further administered 15-days post-resection of the tumor.
E95. The method of E94, wherein the immune checkpoint inhibitor is further administered approximately every two weeks until cessation of further administration.

### Abbreviations

Ad=Ad-RTS-hIL-12 (i.e., a recombinant adenovirus comprising a ligand-inducible gene switch for controlled (ligand induced) transcription and expression (synthesis) of human interleukin-12.
Ad+V=Ad-RTS-hIL-12 and veledimex
AE=adverse event
anti-PD-1=Programmed cell Death protein-1 binding antibody
anti-PD-L1=Programmed cell Death ligand-1 binding antibody
CAP=coactivation protein
CD3=cluster of differentiation 3 protein
CD8=cluster of differentiation 8 protein
CYP3A4=cytochrome P450 3A4
DBD=DNA binding domain
DIPG=Diffuse Intrinsic Pontine Glioma
FoxP3=forkhead box P3 protein (see e.g., Liang Y, et al., “Tumor-infiltrating CD8+ and FOXP3+ lymphocytes before and after neoadjuvant chemotherapy in cervical cancer”, Diagn Pathol. (2018) November 24; 13(1):93 (doi: 10.1186/s13000-018-0770-4) and references cited therein.
GL-261=glioma 261 mouse (murine) model of glioma
hIL-12 (hIL12)=human interleukin-12
IL-12 (IL12)=interleukin-12
kg=kilogram
LTF=ligand-dependent transcription factor
m²=standard measure of subject body surface area
mg=milligram
mIL-12 (mIL12)=murine interleukin-12
mL=milliliter
mOS=median Overall Survival
nivo=nivolumab
PD-1=Programmed cell Death protein-1
PD-L1=Programmed cell Death ligand-1 (a receptor protein)
pg=picogram
PO=per os; by mouth; orally
Q2W=once every 2 weeks
QD=once per day
QDx14=once per day for 14 days
QW=once per week
rGBM=recurrent glioblastoma
RTS=gene switch for ligand-inducible transcriptional activation and expression of operably-linked genes (aka, “RHEOSWITCH® THERAPEUTIC SYSTEM”)
T cell=lymphocyte type of immune cell; typically produced or processed by the thymus gland
Tregs=regulatory T cells
TSP=therapeutic switch promoter
V=veledimex
vp=viral particles
vs=versus
X14=times 14

### ADDITIONAL REFERENCES

AMGEN: “A Phase 1b/3, Multicenter, Open-label Trial of Talimogene Laherparepvec in Combination With Pembrolizumab (MK-3475) for Treatment of Unresected, Stage IIIB to IVM1c Melanoma (MASTERKEY-265)”, 22 Jun. 2015
NATIONAL CANCER INSTITUTE (NCI): “Randomized Phase II/III Study of Nivolumab Plus Ipilimumab Plus Sargramostim Versus Nivolumab Plus Ipilimumab in Patients With Unresectable Stage III or Stage IV Melanoma”, 9 Apr. 2015
Barrett, et al., “Localized Regulated Expression of IL-12 as a Gene Therapy Approach to Cancer Immunotherapy” American Society of Gene & Cell Therapy 19th Annual Meeting (2016); Control/Tracking Number: 2016-A-231-ASGCT
Lebel, et al., “Phase 1 study of intratumoral viral delivery of Ad-RTS-hIL-12+oral veledimex is well tolerated and suggests survival benefit in recurrent high grade glioma”
ASCO Poster, “CTLA-4 Antagonist Monoclonal Antibody for the Treatment of Cancer”, Worldwide Primary Investigator's Meeting (2005)
EP 2591796, Search Report and Opinion dated 21 Mar. 2012
Allard, et al., Clinical Cancer Research, 19(20): 5626-5635 (2013)
Chiocca, et al., American Academy of Neurological Surgery, Slides 1-12; Annual Meeting (2017)
Jackson, et al., Clinical and Developmental Immunology, vol. 2011, Article ID 732413, 20 pages, (2011)
Kim et al., Journal of Immunology Research, vol. 2016, Article ID 4683607, 14 pages
Lasek, et al., Cancer Immunology, Immunotherapy, 63:419-435, (2014)
Melero, et al., Nature Reviews Cancer, 15:457-472 (2015)
Melero, et al., Trends in Immunology, 22(3):113-115 (2001)
Pan, et al., Journal of Immunology, 172:4779-4789 (2004)
Pardoll, Nature Reviews Cancer, 12(4):252-64 (2012)
Patel, et al., Cancers, 6:1953-1985 (2014)
Storz, Ulrich, “Intellectual property issues of immune checkpoint inhibitors”, MAbs. 2016 January; 8(1): 10-26 (Published online 2015 Oct. 14. doi: 10.1080/19420862.2015.1107688).
U.S. Pat. No. 9,815,898
U.S. Pat. No. 9,884,913
U.S. Pat. No. 9,988,452
WO 2004/029197 A3 (PCT/US2002/019898)
WO 2009/048560 A1 (PCT/US2008/011563)
WO 2013/043647 A1 (PCT/US2012/055992)
WO 2013/053775 A1 (PCT/EP2012/070088)
WO 2014/022138 A2 (PCT/US2013/051535)
WO 2014/047350 A1 (PCT/US2013/060716)
WO 2014/189805 A1 (PCT/US2014/038525)
WO 2015/069770 A1 (PCT/US2014/064133)
WO 2015/112900 A1 (PCT/US2015/012754)
WO 2017/019896 A1 (PCT/US2016/044547).

### INCORPORATION BY REFERENCE

The disclosure of all publications and other documents (including issued patents, patent applications, sequence listings therein and therewith, as well as other associated disclosures) and scientific or other articles referenced herein are each hereby incorporated by reference in their entirety. In the event that statements or other information in any such incorporated reference contradicts or conflicts with the present application, the disclosure of the present application shall be dispositive, govern and control.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and the range of equivalency of the claims are intended to be embraced therein.

1-119. (canceled)

120. A method of treating a subject having cancer, comprising administering to the subject:
a. an adenoviral vector, wherein the vector comprises:
i. a first polynucleotide encoding a polypeptide at least 85% identical to wild type human IL-12 p40;
ii. a second polynucleotide encoding a polypeptide at least 85% identical to wild type human IL-12 p35;
iii. a third polynucleotide encoding a VP-16 transactivation domain-retinoic acid-X-receptor fusion protein (VP-16-RXR); and
iv. a fourth polynucleotide encoding a Gal4 DNA binding domain and an ecdysone receptor (EcR) binding domain fusion protein (Gal4-EcR);
b. a diacylhydrazine ligand; and
c. cemiplimab-rwlc.

121. The method of claim 120, further comprising administering to the subject having cancer a corticosteroid, wherein the cumulative amount of corticosteroid administered to the subject is less than or equal to 20 mg during a period of 14 days from the initial dose of the diacylhydrazine ligand.

122. The method of claim 121, wherein the corticosteroid is dexamethasone.

123. The method of claim 120, wherein the diacylhydrazine ligand is veledimex.

124. The method of claim 120, wherein the cancer is a brain cancer.

125. The method of claim 124, wherein the brain cancer is a glioma.

126. A method of treating a subject having cancer, comprising administering to the subject:
a. an Ad-RTS-hIL-12 viral vector, wherein the vector comprises:
i. a first polynucleotide encoding a polypeptide at least 85% identical to wild type human IL-12 p40;
ii. a second polynucleotide encoding a polypeptide at least 85% identical to wild type human IL-12 p35;
iii. a third polynucleotide encoding a VP-16 transactivation domain-retinoic acid-X-receptor fusion protein (VP-16-RXR); and
iv. a fourth polynucleotide encoding a Gal4 DNA binding domain and an ecdysone receptor (EcR) binding domain fusion protein (Gal4-EcR);
b. a diacylhydrazine ligand; and
c. nivolumab.

127. The method of claim 126, further comprising administering to the subject having cancer a corticosteroid, wherein the cumulative amount of corticosteroid administered to the subject is less than or equal to 20 mg during a period of 14 days from the initial dose of the diacylhydrazine ligand.

128. The method of claim 127, wherein the corticosteroid is dexamethasone.

129. The method of claim 126, wherein the diacylhydrazine ligand is veledimex.

130. The method of claim 126, wherein the cancer is a brain cancer.

131. The method of claim 130, wherein the brain cancer is a glioma.

132. A method of treating a pediatric subject having cancer, comprising administering to the subject:
a. an adenoviral vector, wherein the vector comprises:
i. a first polynucleotide encoding a polypeptide at least 85% identical to wild type human IL-12 p40;
ii. a second polynucleotide encoding a polypeptide at least 85% identical to wild type human IL-12 p35;
iii. a third polynucleotide encoding a VP-16 transactivation domain-retinoic acid-X-receptor fusion protein (VP-16-RXR); and
iv. a fourth polynucleotide encoding a Gal4 DNA binding domain and an ecdysone receptor (EcR) binding domain fusion protein (Gal4-EcR); and
b. a diacylhydrazine ligand.

133. The method of claim 132, further comprising administering to the subject a programmed cell death protein 1 (PD1) inhibitor.

134. The method of claim 133, wherein the PD1 inhibitor is selected from the group consisting of cemiplimab-rwlc, nivolumab, pembrolizumab, MEDI0680, pidilizumab, BGB-A317, spartalizumab, and STI-A1110.

135. The method of claim 133, wherein the PD1 inhibitor is cemiplimab-rwlc.

136. The method of claim 133, wherein the PD1 inhibitor is nivolumab.

137. The method of claim 132, wherein the diacylhydrazine ligand is veledimex.

138. The method of claim 132, further comprising administering to the pediatric subject having cancer a corticosteroid, wherein the cumulative amount of corticosteroid administered to the subject is less than or equal to 20 mg during a period of 14 days from the initial dose of the diacylhydrazine ligand.

139. The method of claim 138, wherein the corticosteroid is dexamethasone.

140. The method of claim 132, wherein the cancer is a brain cancer.

141. The method of claim 140, wherein the brain cancer is a glioma.

142. The method of claim 141, wherein the glioma is diffuse intrinsic pontine glioma (DIPG).

143. A method of treating a subject having cancer, comprising administering to the subject:
a. an adenoviral vector, wherein the vector comprises:
i. a first polynucleotide encoding a polypeptide at least 85% identical to wild type human IL-12 p40;
ii. a second polynucleotide encoding a polypeptide at least 85% identical to wild type human IL-12 p35;
iii. a third polynucleotide encoding a VP-16 transactivation domain-retinoic acid-X-receptor fusion protein (VP-16-RXR); and
iv. a fourth polynucleotide encoding a Gal4 DNA binding domain and an ecdysone receptor (EcR) binding domain fusion protein (Gal4-EcR);
b. a diacylhydrazine ligand; and
c. a corticosteroid, wherein the cumulative amount of corticosteroid administered to the subject is less than or equal to 20 mg during a period of 14 days from the initial dose of the diacylhydrazine ligand.

144. The method of claim 143, further comprising administering to the subject having cancer a programmed cell death protein 1 (PD1) inhibitor.

145. The method of claim 144, wherein the PD1 inhibitor is selected from the group consisting of cemiplimab-rwlc, nivolumab, pembrolizumab, MEDI0680, pidilizumab, BGB-A317, spartalizumab, and STI-A1110.

146. The method of claim 144, wherein the PD1 inhibitor is cemiplimab-rwlc.

147. The method of claim 144, wherein the PD1 inhibitor is nivolumab.

148. The method of claim 143, wherein the corticosteroid is dexamethasone.

149. The method of claim 143, wherein the diacylhydrazine ligand is veledimex.

150. The method of claim 143, wherein the cancer is a brain cancer.

151. The method of claim 150, wherein the brain cancer is a glioma.


SEQUENCE LISTING (18 sequences)

SEQ ID NO: 1 - DNA, 648 bp, Mus musculus
atgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctga

SEQ ID NO: 2 - DNA, 1008 bp, Mus musculus
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctag

SEQ ID NO: 3 - DNA, 660 bp, Homo sapiens
atgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgacagagtgacgagctatctgaatgcttcctaa

SEQ ID NO: 4 - DNA, 987 bp, Homo sapiens
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttag

SEQ ID NO: 5 - protein, 215 aa, Mus musculus
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 6 - protein, 335 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 7 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 8 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 9 - protein, 878 aa, Drosophila melanogaster
MKRRWSNNGGFMRLPEESSSEVTSSSNGLVLPSGVNMSPSSLDSHDYCDQDLWLCGNESGSFGGSNGHGLSQQQQSVITLAMHGCSSTLPAQTTIIPINGNANGNGGSTNGQYVPGATNLGALANGMLNGGFNGMQQQIQNGHGLINSTTPSTPTTPLHLQQNLGGAGGGGIGGMGILHHANGTPNGLIGVVGGGGGVGLGVGGGGVGGLGMQHTPRSDSVNSISSGRDDLSPSSSLNGYSANESCDAKKSKKGPAPRVQEELCLVCGDRASGYHYNALTCEGCKGFFRRSVTKSAVYCCKFGRACEMDMYMRRKCQECRLKKCLAVGMRPECVVPENQCAMKRREKKAQKEKDKMTTSPSSQHGGNGSLASGGGQDFVKKEILDLMTCEPPQHATIPLLPDEILAKCQARNIPSLTYNQLAVIYKLIWYQDGYEQPSEEDLRRIMSQPDENESQTDVSFRHITEITILTVQLIVEFAKGLPAFTKIPQEDQITLLKACSSEVMMLRMARRYDHSSDSIFFANNRSYTRDSYKMAGMADNIEDLLHFCRQMFSMKVDNVEYALLTAIVIFSDRPGLEKAQLVEAIQSYYIDTLRIYILNRHCGDSMSLVFYAKLLSILTELRTLGNQNAEMCFSLKLKNRKLPKFLEEIWDVHAIPPSVQSHLQITQEENERLERAERMRASVGGAITAGIDCDSASTSAAAAAAQHQPQPQPQPQPSSLTQNDSQHQTQPQLQPQLPPQLQGQLQPQLQPQLQTQLQPQIQPQPQLLPVSAPVPASVTAPGSLSAVSTSSEYMGGSAAIGPITPATTSSITAAVTASSTTSAVPMGNGVGVGVGVGGNVSMYANAQTAMALMGVALHSHQEQLIGGVAVKSEHSTTA

SEQ ID NO: 10 - protein, 440 aa, Artificial Sequence
  Nivolumab heavy chain sequence
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence
  Nivolumab light chain sequence
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 335 aa, Artificial Sequence; identical to SEQ ID NO: 6
  IL-12 p40

SEQ ID NO: 13 - DNA, 1008 bp, Artificial Sequence
  IL-12 p40
atgtgcccccagaagctgaccatcagctggttcgccatcgtgctgctggtgagccccctgatggccatgtgggagctggagaaggacgtgtacgtggtggaggtggactggacccccgacgcccccggcgagaccgtgaacctgacttgcgacacccccgaggaggacgacatcacctggaccagcgaccagagacacggcgtcatcggcagcggcaagaccctgaccatcaccgtgaaggagttcctggacgccggacagtacacctgtcacaagggcggcgagaccctgagccacagccacctgttgctgcacaagaaggagaacggcatctggagcaccgagatcctgaagaacttcaagaacaagaccttcctgaagtgcgaggcccccaactacagcggcagattcacctgtagctggctggtgcagagaaacatggacctgaagttcaacatcaagagcagcagcagcagccccgacagcagagccgtgacatgcggcatggccagcctgagcgccgagaaggtgaccctggaccagagagactacgagaagtacagcgtgagctgccaggaggacgtgacctgtcccaccgccgaggagaccctgcccatcgagcttgccctggaagccagacagcagaacaagtacgagaactacagcaccagcttcttcatcagagacatcatcaagcccgacccccccaagaacctccagatgaagcccctgaagaacagccaggtggaggtgtcctgggagtaccccgacagctggagcaccccccacagctacttcagcctgaagttcttcgtgagaatccagagaaagaaggagaagatgaaggagaccgaggagggctgcaaccagaagggcgctttcctggtggagaaaaccagcaccgaggtgcagtgcaagggcggcaacgtgtgtgtgcaggcccaggacagatactacaacagcagctgctccaagtgggcctgcgtgccctgccgcgtgagaagctga

SEQ ID NO: 14 - protein, 215 aa, Artificial Sequence; identical to SEQ ID NO: 5
  IL-12 p35

SEQ ID NO: 15 - DNA, 648 bp, Artificial Sequence
  IL-12 p35
atgtgccagagcagatacctgttgttcctggctaccctggccctgctgaaccacctgagcctggcccgcgtgatccccgtgagcggccccgccagatgcctgagccagagcagaaacctgttgaaaacaaccgacgacatggtgaaaaccgccagagagaagctgaagcactacagctgcaccgccgaggacatcgaccacgaggacatcaccagagaccagaccagcaccctgaaaacctgtctgcccctggagctgcacaagaacgagagctgcctggctaccagagagaccagcagcaccaccagaggcagctgcctgcccccccagaaaaccagcctgatgatgaccctgtgcctgggcagcatctacgaggacctgaagatgtaccagaccgagttccaggccatcaacgccgccctgcaaaaccacaaccaccagcagatcatcctggacaagggcatgttggtggccatcgacgagctgatgcagagcctgaaccacaacggcgagaccctgagacagaagccccccgtgggcgaggccgacccctacagagtgaagatgaagctgtgcatcctgctgcacgccttcagcaccagagtggtgaccatcaacagagtgatgggctacctgagcagcgcctga

SEQ ID NO: 16 - protein, 17 aa, Artificial Sequence
  natural ecdysone receptor
RRGGTTCANTGACACYY

SEQ ID NO: 17 - DNA, 13 bp, Artificial Sequence
  natural ecdysone receptor
  misc_feature (7)..(7): where N can refer to any naturally occurring nucleotide
aggtcanaggtca

SEQ ID NO: 18 - protein, 15 aa, Artificial Sequence
  natural ecdysone receptor
GGGTTGAATGAATTT